(19) United States
(12) Patent Application Publication
(10) Pub. No.: US 2011/0092566 A1
(43) Pub. Date: Apr. 21, 2011

(54) Treatment of cancer with aldose reductase inhibitors

(76) Inventors: Satish K. Srivastava, Galveston, TX (US); Kota V. Ramana, Galveston, TX (US)

(21) Appl. No.: 12/807,033

(22) Filed: Aug. 26, 2010

Related U.S. Application Data
(63) Continuation-in-part of application No. 12/308,915, filed on Aug. 11, 2009.
Application No. 12/308,915 is a continuation of application No. 11/478,069, filed on Jun. 29, 2006, now abandoned.
Application No. 11/478,069 is a continuation-in-part of application No. 11/282,801, filed on Nov. 18, 2005, now Pat. No. 7,702,430.
(60) Provisional application No. 60/629,448, filed on Nov. 19, 2004.

(51) Int. Cl. A61K 31/7088; A61K 31/351; A61K 31/335; A61K 31/135; A61K 31/69; A61P 35/00

(52) U.S. Cl. 514/44 A; 514/549; 514/449; 514/651; 514/64

(57) ABSTRACT

Provided herein are methods of treating a pathophysiological state or symptoms thereof resulting from aldose reductase-mediated signaling in a cytotoxic pathway in a subject using an inhibitor of aldose reductase. Particularly, specific inhibitors may be small molecules such as fidarestat or siRNA. Also, methods of treating breast and prostate cancers or suppressing metastasis of colon cancer thereof using the siRNAs and aldose reductase inhibitors are provided.

### CROSS-REFERENCE TO RELATED APPLICATIONS

This is a continuation-in-part of pending U.S. Ser. No. 12/308,915, which is a U.S. national stage application under 35 U.S.C. §371 of PCT/US2007/015322, filed Jun. 29, 2007, now abandoned, which claims benefit of priority under 35 U.S.C. §120 of continuation-in-part application U.S. Ser. No. 11/478,069, filed Jun. 29, 2006, now abandoned, which claims benefit of priority under 35 U.S.C. §120 of pending non-provisional application U.S. Ser. No. 11/282,801, filed Nov. 18, 2005, now U.S. Pat. No. 7,702,430, which claims benefit of priority under 35 U.S.C. §119(e) of provisional U.S. Ser. No. 60/629,448, filed Nov. 19, 2004, now abandoned, the entirety of which applications are hereby incorporated by reference.

### FEDERAL FUNDING LEGEND

This invention was produced in part using funds obtained through Grant CA129383 from the National Institutes of Health. Consequently, the federal government has certain rights in this invention.

### BACKGROUND OF THE INVENTION

1. Field of the Invention

The present invention relates generally to the fields of enzymology, protein structure and drug screening. More specifically, the present invention relates to the use of inhibitors of aldose reductase to treat pathophysiological states involving aldose reductase signaling.

2. Description of the Related Art

Aldose reductase is a monomeric (a/b)₈-barrel (TIM barrel) protein belonging to the aldo-keto reductase (AKR) superfamily (1-3). Aldose reductase is a broad-specificity oxidoreductase catalyzing the reduction of a structurally-diverse range of aldehydes, including medium to long chain aldehydes, glucose and other aldo-sugars, aldehyde metabolites of neurotransmitters, isocorticosteroid hormones, and a variety of xenobiotic aldehydes to their corresponding alcohols (4). Reduction of glucose to sorbitol by aldose reductase constitutes the first and rate-limiting step of the polyol pathway that converts glucose to fructose via sorbitol dehydrogenase. Although this pathway usually represents a minor route of glucose metabolism, its activation during diabetes has been linked to the development of several clinically significant secondary complications such as nephropathy, neuropathy, retinopathy and cardiovascular related complications (4, 5). Several drugs that inhibit aldose reductase have been shown to prevent hyperglycemia-induced changes in nerve, kidney, and lens of experimental animals, although clinical trials with Type I and Type II diabetics have not been uniformly positive (4-6). Representative examples of aldose reductase inhibitors include sorbinil, tolrestat, zopolrestat and fidarestat as well as genetic ablation by siRNA.

In addition to glucose, it has been shown that aldose reductase catalyzes the reduction of multiple biologically-active aldehydes generated by the peroxidation of membrane lipids and lipoproteins (7-9) or during glucose (10) and amine (11) metabolism. The aldehyde-detoxifying role of aldose reductase is supported by the observation that inhibition of the enzyme increases the accumulation of lipid peroxidation products (12, 13) that cause cytotoxicity (14, 15). The most abundant and toxic lipid peroxidation product is 4-hydroxy-trans-2-nonenal (16) which is reduced by aldose reductase in vitro and in vivo.

A primary role of aldose reductase in aldehyde detoxification is consistent with its structure. The active site of the enzyme is highly hydrophobic and contains few polar residues typically required for binding sugars with high specificity and affinity (2, 3). These features are, however, compatible with binding to hydrophobic lipid-derived aldehydes. Additionally, the substrate-specificity of aldose reductase is unusually broad, in part because the enzyme derives most of the energy required to achieve a substrate transition state from cofactor-binding (17). The active site environment exerts low stabilization on the transition state (18). Furthermore, it has been demonstrated recently that aldose reductase-catalyzed products mediate cytokine, chemokine, growth factor, and hyperglycemia-induced signaling that activates NF-kB and AP1, and regulates vascular epithelial cell (VEC) and human lens epithelial cell (HLEC) apoptosis, and vascular smooth muscle cell (VSMC) proliferation (15, 21, 22).

The range of aldehydes recognized by the aldose reductase active site is increased further by the ability of the enzyme to bind glutathione-aldehyde conjugates (19, 20), such as glutathionyl HNE. Given the high concentration of reduced glutathione in most cells and the highly electrophilic nature of several aldose reductase substrates, it is possible that reduction of aldehyde-glutathione conjugates, in addition to free aldehydes, may be a primary in vivo function of aldose reductase and that glucose may be an incidental substrate of the enzyme. Previous kinetic studies showed that glutathiolation increases the catalytic efficiency with which unsaturated aldehydes are reduced by aldose reductase (19), suggesting that the active site of aldose reductase contains a specific glutathione-binding domain (20). Nevertheless, the precise nature of glutathione binding to aldose reductase remained unclear.

It was discovered that vascular smooth muscle cell growth in culture was significantly inhibited by aldose reductase inhibitors. In 2002 this provided, for the first time, the idea that aldose reductase inhibitors could be antiproliferative. Simultaneously, it was demonstrated that inhibition of aldose reductase also inhibited apoptosis of vascular endothelial cells and lens epithelial cells induced by cytokines such as TNF-a or by bacterial lipopolysaccharides (LPS). Later on, it was shown that aldose reductase (AR) inhibition or ablation significantly prevented the synthesis and release of various cytokines including TNF-α, chemokines, and growth factors as well as Cox by human macrophages. While trying to understand the mechanism, it was discovered that AR-catalyzed lipid aldehyde-glutathione product mediated the LPS-signal that activated the transcription factors nuclear factor kappa beta (NFκB) and activator protein 1 (AP-1) which are known to transcribe inflammatory markers. Aldose reductase inhibitor inhibited the increase in various cytokines, chemokines, Cox and growth factors by 75 to 100% and prevented the decrease in cardiac contractibility. Overall, aldose reductase inhibition prevented mulit-organ failure as normally observed in septic shock and is the major cause of death. These results were subsequently confirmed in cecal-ligation and perforation mice model.

Since increase in inflammatory markers and growth factors is known to be the major cause of proliferation of cancer cells resulting in growth and Cox-2 inhibitors are used in the prevention and management of colon cancer, the effect of aldose reductase inhibitors in the proliferation of various human cancer cells such as Caco-2 cells in culture have been studied. Results showed that aldose reductase inhibitors, sorbinil, tolrestat, and zopolrestat significantly prevented the growth of immortalized human colon cancer cell line SW-480, HT29 and HCT-116 induced by growth factors. These results demonstrated that all of the above mentioned inhibitors including fidarestat as well as genetic ablation by siRNA significantly prevented the growth factor-induced cell growth of SW-480 cells, suggesting that these inhibitors could be used as anti-colon cancer drugs.

Although aldose reductase inhibitors seem to have potential as cancer therapeutic agents, only a few of the inhibitors have been demonstrated to be effective. Aldose reductase inhibitors also have potential to treat cancers varying in their etiology and pathophysiology. More specifically, it is still largely unknown whether aldose reductase inhibitors may be used to treat cancers such as colon cancer, breast cancer and prostate cancer. The effect that aldose reductase inhibitors have on the spread and metastasis of these cancers is also largely unknown. Furthermore, the use of fidarestat as the aldose reductase inhibitor to treat proliferative disorders has not been demonstrated. Therefore, the prior art is deficient in using aldose reductase inhibitors as therapeutic agents in the treatment of cell proliferative disease. The prior art is also deficient in using aldose reductase inhibitor to treat colon cancer, breast cancer and prostate cancer. This treatment includes limiting the spread and metastasis of the above mentioned cancers. The present invention fulfills this long-standing need and desire in the art.

### SUMMARY OF THE INVENTION

The present invention is directed to a crystalline structure of a ternary AR:NADPH:glutathione-like ligand complex. The crystalline structure diffracts x-rays for determining atomic co-ordinates of said complex with a resolution of about 3 Å to about 1.94 Å. The glutathione-like ligand interacts with both a glutathione binding domain and a carbonyl binding site within an active pocket formed by an AR:NADPH complex within the ternary structure. The present invention also is directed to a related crystalline structure comprising a ternary AR:NADPH:DCEG complex diffracts x-rays for determining atomic co-ordinates of the complex with a resolution of about 1.94 Å.

The present invention also is directed to a method of designing a potential inhibitor of glutathione-aldehyde conjugate binding to aldose reductase. The method comprises identifying a glutathione-like ligand that interacts with the glutathione binding domain, but does not block the carbonyl binding site, in the active pocket of an aldose reductase which has the three-dimensional conformation determined by DCEG binding to AR:NADPH. The identification of the potential inhibitor is based at least in part on a computer model of the crystalline AR:NADPH:DCEG ternary structure described herein.

The present invention is directed to a related method of screening for inhibitors of glutathione-aldehyde conjugate reduction by aldose reductase. The method comprises using the crystalline ternary structure described herein to design a potential inhibitor that binds to the glutathione binding domain in aldose reductase, but does not interfere with the carbonyl binding site. The design is based in part on computer modeling of the crystalline AR:NADPH:DCEG. The aldose reductase is complexed with the potential inhibitor and the aldose reductase:inhibitor complex is contacted with a lipid aldehyde and with the lipid aldehyde conjugated to glutathione. Detection of a reduced lipid aldehyde product, but not a reduced glutathione-lipid aldehyde product, screens for the inhibitor.

The present invention is directed further to the specific inhibitors of glutathione-aldehyde conjugate reduction designed and screened for by the methods described herein.

The present invention is directed further yet to a method of preventing a pathophysiological state or treating symptoms thereof resulting from aldose-reductase mediated signaling of a cytotoxic pathway in a subject. The method comprises administering a pharmacologically effective amount of the inhibitors of glutathione-aldehyde conjugate reduction described herein to the subject and inhibiting the reduction of a glutathione-aldehyde substrate via aldose reductase to prevent cytotoxic signaling in the subject. The cytotoxic signals could be generated by cytokines, chemokines, reactive oxygen species, endotoxins, growth factors, hyperglicemia and biologically active agents, e.g., bioterrorism agents.

The present invention is directed further still to a related method of treating a pathophysiological state or symptoms thereof resulting from aldose-reductase-mediated signaling in a cytotoxic pathway in a subject. The method comprises administering a pharmacologically effective amount of an inhibitor of aldose reductase to the subject thereby preventing aldose reductase mediated signaling. The aldose reductase inhibitor may be a small interfering RNA (siRNA) or an inhibitor that is effective to inhibit reduction of a glutathione-aldehyde conjugate by aldose reductase.

The present invention is directed further still to another related method of treating colon cancer in a subject. The method comprises administering a pharmacologically effective amount of an aldose reductase small interfering RNA (siRNA) or aldose reductase inhibitor to the subject to inhibit colon cancer cell proliferation thereby treating the colon cancer. The present invention is directed to a related method further comprising suppressing metastasis of the cancer to a metastatic cancer.

The present invention is directed further still to a method of suppressing metastasis of a cancer cell in a subject. The method comprises inhibiting aldose reductase activity within the cancer cell to prevent migration thereof through an extracellular matrix thereby suppressing metastasis of the cancer cell. Contacting the cancer cell with effective amounts of an siRNA or other aldose reductase inhibitor prevents migration of the cancer cell through an extracellular matrix.

Other and further aspects, features, and advantages of the present invention will be apparent from the following description of the embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate embodiments of the invention and therefore are not to be considered limiting in their scope.

FIGS. 1A-1B depict the structure of DCEG and human aldose reductase. FIG. 1A is the DCEG structure showing hydrogen bond interactions with aldose reductase and solvent as hashed lines. The dashed semi-circles denote hydrophobic interactions with the protein. FIG. 1B is a ribbon drawing of AR:NADPH (arrows) with DCEG bound (ball-n-stick), the β-strands in the (α/β)₈ barrel and the mobile active loops A, B, and C.

FIGS. 2A-2B depict the human aldose reductase active site with DCEG bound. FIG. 2A shows a top view of the human aldose reductase molecular surface (purple) with the active site occupied by DCEG (yellow). For clarity, solvent atoms have been omitted. FIG. 2B shows a close-up view of DCEG (yellow ball-stick) and the two waters bound in the aldose reductase active site. The active site residues: Tyr-48, His-110, Trp-110, and NADPH, sit at the base of the deep cleft where the DCEG dicarboxyethyl moiety is bound. Coloring is the same as for FIG. 1B.

FIG. 3 is a model of a potential GS-like inhibitor with an aldehyde bound in the active site. An aldehyde chain (gold, green, or blue) may pass though one of three channels between the inhibitor (yellow) and protein (purple) to reach the aldose reductase active site. The mobile loops A, B, and C are colored as in FIGS. 1B and 2.

FIGS. 4A-4C illustrate the regulation of high glucose-induced TNF-a production by aldose reductase. Growth-arrested VSMC in 5.5 mM glucose (NG) were preincubated for 1 h without or with apocyanin (25 mM), D609 (100 mM), calphostin C (0.2 mM), N-acetyl cysteine (10 mM) and NF-kB inhibitor (18 mM) (FIG. 4A) and without or with sorbinil or tolrestat (10 mM each) (FIG. 4B) followed by the addition of glucose (19.5 mM) and incubation for the indicated times. Aldose reductase antisense ablated VSMC were incubated with HG for the indicated times (FIG. 4C). The data represent mean±SEM (n=4). **P<0.001 versus cells incubated in high glucose.

FIGS. 5A-5F illustrate the effect of aldose reductase inhibition/ablation on LPS- and lipid aldehyde-induced signaling in RAW264.7 cells. Cells were growth-arrested in Dulbecco's modified Eagle's medium containing 0.1% serum with or without sorbinil (10 mM) and challenged with LPS (1 mg/ml). At the indicated times, cells were harvested for measurement of HNE (FIG. 5A), protein-HNE adducts (FIG. 5B), NF-kB (FIG. 5C), and TNF-a and IL-6 (FIG. 5D) as described in the methods. Cells were growth arrested as described above or transfected with control or aldose reductase siRNA oligonucleotides, incubated with GS-HNE-ester, or GS-DHN-ester (1 mM), and harvested for determination of NF-kB (FIG. 5E), membrane-bound total PKC (FIG. 5F).

FIGS. 6A-6E illustrate the effect of aldose reductase inhibition on LPS-induced cytokines. C57BL/6 mice (N=6 per group) were injected with sorbinil or vehicle for 3 days, and then challenged with LPS (4 mg/kg). At the indicated times, TNF-a, IL-6, IL-12, interferon (IFN)-g, IL-1b, and monocyte chemo attractant protein (MCP)-1 levels in serum and in heart homogenates were determined (FIGS. 6A-6D). Prostaglandin E2 (PGE-2), cyclo-oxygenase 2 (COX-2), and nitrate levels were measured separately (FIG. 6E). Solid symbols, values from the mice injected with LPS; open symbols, values from the mice treated with LPS and sorbinil.

FIGS. 7A-7D illustrate the effect of aldose reductase inhibition on LPS-induced cardiac dysfunction. C57BL/6 mice (N=6 per group) were treated as described in FIG. 9, and fractional shortening percent (FS %) was determined by M-mode echocardiography 0-48 h after LPS injection (FIG. 7A). Values are means of SD. The data were analyzed by one-way repeated-measures ANOVA. Cardiac function in isolated mouse hearts (Langendorff preparation) was determined at various times after LPS challenge as a function of increasing Ca2+ concentration (FIG. 7B) or coronary flow rate (FIG. 7C). Values are means±SEM of six independent experiments. FIG. 7D shows the protective effect of aldose reductase inhibition on LPS-induced lethality as percent (%) survival of mice 48 h after LPS administration at increasing doses or LPS plus sorbinil administration either 24 h before LPS (pre-treatment) or 2 h after LPS (post-treatment). The LD50 for LPS alone was 14 mg/kg, that with sorbinil pre-treatment was 24 mg/kg, and with sorbinil post-treatment was 20 mg/kg (N=8 per group, *p<0.001).

FIGS. 8A-8D illustrate the effect of aldose reductase inhibition on LPS signaling in the heart. C57BL/6 mice (N=6 per group) were treated as described in FIG. 6E. At the indicated times, NF-kB activation (FIG. 8A), AP1 activation with an electrophoretic mobility shift assay (FIG. 8B), iNOS expression by western blotting (FIG. 8C), or PKC activation with a total PKC assay system (FIG. 8D) (SignaTect, Promega), or phosphorylated forms of the indicated kinases by western blotting (FIG. 8E), was detected. Values are means±SEM (N=4). *P<0.001 versus LPS-treated mice. OD, optical density.

FIGS. 9A-9G illustrate that inhibition or ablation of aldose reductase prevents growth factor-induced PGE2 production and Cox-2 expression in colon cancer cells. Growth-arrested Caco-2 cells were pre-incubated with sorbinil or carrier for 24 h (FIG. 9A) and with aldose reductase antisense or scrambled oligos (FIG. 9B). The inset in FIG. 9B represents Western blot analysis for aldose reductase protein in untransfected (c), scrambled (s) and aldose reductase antisense (a) oligo transfected cell extracts. The aldose reductase inhibited and ablated cells were stimulated with BFGF or PDGF as in FIG. 10A except that Cox activity was measured by a Cox activity assay kit (FIG. 9C). Western blots were developed using antibodies against Cox-2 (FIG. 9D), Cox-1 (FIG. 9E) and GAPDH (FIG. 9F). FIG. 9G is a densitometric analysis of FIG. 9D. Bars represent mean±S.E. (n=4); # p<0.001 compared with treatment without the inhibitor or scrambled oligo transfected cells and * p<0.01; **, p<0.001 compared with growth factor treated cells.

FIGS. 10A-10D illustrate the effects of aldose reductase inhibitors on HT29 and A549 cell proliferation, induction of growth factors and protein expression. FIGS. 10A-10B demonstrate that sorbinil and zopolrestat and sorbinil and tolrestat inhibit HT29 and A549 cell proliferation, respectively. FIG. 10C demonstrates that sorbinil and zopolrestat inhibited production of PGE2 by HT29 cells. FIG. 10D demonstrates that sorbinil and tolrestat inhibit Cox-2 and iNOS expression in A549 cells.

FIGS. 11A-11D illustrate that inhibition of aldose reductase prevents growth factor-induced Cox-2 mRNA expression and NF-kB in colon cancer cells. Growth-arrested Caco-2 cells were pre-incubated with sorbinil or carrier for 24 h followed by stimulation with of BFGF or PDGF for 3 h. FIG. 11A-11B measure Cox-2 and β-actin expression, respectively. FIG. 11C is a densitometric analysis of FIG. 11A. FIG. 11D shows NF-kB-dependent reporter SEAP activity. The Inset in FIG. 11D shows the chemiluminescence of SEAP. FIG. 11E shows NF-kB activity. Bars represent mean±S.E. (n=4); # p<0.01 as compared to control cells. * p<0.01 compared cells treated with growth factors.

FIGS. 12A-12F illustrate that the inhibition of aldose reductase abrogates growth factor-induced PKC activation and growth in colon cancer cells. Quiescent Caco-2 cells were preincubated with sorbinil for 24 h followed by stimulation with BFGF or PDGF for 3 h. FIG. 12A shows membrane-bound PKC activity. Western blot analysis using antibodies against phsopho-PKC-b2 (FIG. 12B) and GAPDH (FIG. 12C) are depicted. FIG. 12D is a densitometric analysis of FIG. 12B. Growth-arrested Caco-2 cells were pre-incubated with or without sorbinil or tolrestat (FIG. 12E) or were transfected with aldose reductase antisense oligo followed by stimulation with BFGF or PDGF for 24 h and cell viability was measured by MTT assay (FIG. 12F). Bars represent mean±S.E. (n=4); # p<0.01 as compared to control cells. * p<0.01 compared to cells treated with growth factors.

FIGS. 13A-13C illustrate the effects of sorbinil on the cell cycles of Caco-2 cells and A549 cells. FIGS. 13A-13B demonstrate that sorbinil prevents the synthesis S phase of the cell cycle in Caco-2 colon cancer cells and in A549 lung cancer cells, respectively. FIG. 13C demonstrates that sorbinil and tolrestat prevented the G1/S phase related proteins expression.

FIGS. 14A-14B illustrate the effect of PKC, NF-kB and Cox-2 inhibitors and aldose reductase inhibitors on growth factor-induced PGE2 and ROS production, respectively in colon cancer cells. Growth-arrested Caco-2 cells were pre-incubated with PKC, NF-kB and Cox-2 inhibitors or ROS scavenger for 30 min (FIG. 14A) or aldose reductase inhibitors for 24 h (FIG. 14B). The growth-arrested Caco-2 cells were incubated further with BFGF or PDGF for 24 h (FIG. 14A) and 1 h (FIG. 14B). Bars represent mean±S.E. (n=4); # p<0.001 Vs. control cells and * p<0.01 Vs. cells treated with growth factors.

FIGS. 15A-15D illustrate the effect of AR-catalyzed reaction products on PGE2 and Cox-2 in colon cancer cells. The growth-arrested Caco-2 cells preincubated without or with sorbinil for 24 h were incubated with HNE, GS-HNE- or GS-DHN-esters for 24 h. FIG. 15A illustrates PGE2 production. Western blots were developed using antibodies against Cox-2 (FIG. 15B) and GAPDH (FIG. 15C). FIG. 15D is a densitometric analysis of FIG. 15B. Bars represent mean±S.E. (n=4); # p<0.001 Vs. control cells and * p<0.01 Vs. cells treated with aldehydes.

FIGS. 16A-16B illustrate that sorbinil and zopolrestat separately inhibit penetration of an extracellular matrix material by HT29 cells (FIG. 16A) and by A549 cells (FIG. 16B).

FIGS. 17A-17B illustrate the effect of aldose reductase siRNA on tumor size of SW480 xenografts (FIG. 17A) and on body weight (FIG. 17B). At different days tumors were measured in two dimensions using calipers.

FIGS. 18A-18D illustrate the effects of sorbinil on the number of aberrant crypt foci (ACF) in azoxymethane (AOM) treated wild type and knock out mice. FIG. 18A demonstrates the difference between normal crypts and ACFs using light microscopy. FIG. 18B compares ACF formation in azoxymethane mice with or without sorbinil. FIG. 18C compares ACF formation in the presence of sorbinil in azoxymethane-treated mice and Knockout (KO) mice. FIG. 18D demonstrates that sorbinil inhibits azoxymethane-induced Cox-2 and iNOS expression in mice.

FIGS. 19A-19B illustrate the effects of sorbinil on induced HT29 metastatic tumor growth in a nude mouse liver model. FIG. 19A shows bioluminescent expression of green fluorescent protein (GFP) in metastic HT29 tumors with and without sorbinil. FIG. 19B converts the bioluminescence in FIG. 19A to pixel numbers.

FIGS. 20A-20B illustrate that the growth of A540 xenografts in nu/nu nude mice was arrested in the presence of the aldose reductase inhibitor zopolrestat. Treatment was started when the tumor cross-sectional area was ˜45 mm2 and continued for 29 days. Tumors were measured in two dimensions using calipers.

FIGS. 21A-21C illustrate that siRNA inhibition of aldose reductase prevents tumorigenesis and production of reactive oxygen species in A549 lung cancer xenografts. FIG. 21A shows the tumor cross-sectional area of At549 tumors in control and siRNA treated mice. FIGS. 21B-21C are histological sections of A549 xenografts from control and siRNA treated mice demonstrating inhibition of aldose reductase expression and ROS production, respectively.

FIG. 22A-22B shows inhibition of aldose reductase prevents GF-induced breast cancer MCF-7 cell growth. Growth-arrested MCF-7 cells with or without aldose reductase inhibitor, (FIG. 22A) sorbinil or tolrestat (10 μM) or (FIG. 22B) fidarestat (fid) were incubated with IGF or PDGF for 24 h. Cell viability was determined by MTT assay. Bars represent means±SE (n=4); #p<0.001 Vs. control cells. *p<0.01 Vs. cells treated with GF.

FIG. 23A-23D show aldose reductase inhibition prevents estogen independent breast cancer cell growth. Estrogen independent breast cancer cells were treated with and without aldose reductase inhibitors (fidarestat, 5 uM) along with 10-10000 nM concentrations of various chemotherapeutic drugs for 72 hrs and cell growth was measured by MTT assay. Percentage of cell growth was calculated using appropriate controls.

FIG. 24A-24B show inhibition of aldose reductase prevents androgen- and GFs-induced proliferation in prostate cancer cells. Growth-arrested (FIG. 24A) LNCaP cells and (FIG. 24B) PC3 cells were pre-incubated with fidarestat (Fid; 5 μM) or carrier for 24 h followed by stimulation with androgen R1881 (1 nM) or 5% fetal bovine serum (FBS) or IGF-I (50 ng/ml) or EGF (20 ng/ml) for another 24 h. Cell viability was determined by MTT assay. Bars represent mean±S.E. (n=4); * p<0.001 compared to control and **, p<0.01 compared to cells treated with stimulant.

FIG. 25 shows inhibition of aldose reductase prevents fetal bovine serum (FBS)-induced androgen receptor expression in LNCaP cells. Growth-arrested LNCaP cells were pre-incubated with fidarestat (5 μM) or carrier for 24 h followed by stimulation with FBS for another 24 h. The pooled cell extracts from 3 independent experimental sets were subjected to SDS-PAGE and Western blots were developed using antibodies against androgen receptor and GAPDH.

FIG. 26 shows inhibition of aldose reductase prevents PSA secretion in LNCaP cells. Growth-arrested LNCaP cells (3×10⁵/well) in 6 well plate were pre-incubated with fidarestat (2.5 μM) or carrier for 24 h followed by stimulation with RPMI medium containing 5% FBS for another 24 h with varying concentrations of fidarestat. PSA was measured in culture medium using ELISA kit. Bars represent mean±S.E. (n=4); **, p<0.01; * p<0.001 compared to with out inhibitor.

FIG. 27 shows the effect of aldose reductase inhibition on PC3 xenografts. An aliquot of 1×10⁶ PC3 cells in matrigel matrix with PBS with 1;1 ratio of 100 μL was injected s.c. into one flank of each nu/nu nude mouse (n=6). After 5 days of implantation of PC3 cells, animals were treated with fidarestat (50 mg/kg body weight) in drinking water. At different days, tumors were measured in two dimensions using calipers.

FIG. 28 shows the downstream signaling of Reactive Oxygen Species (ROS)-induced and AR-catalyzed reaction products mediate cytokine, chemokine, growth factor and carcinogen-induced signals that promote cell growth and metastasis.

FIG. 29A-29D shows that inhibition of aldose reductase prevents growth factors-induced invasion and migration of HT29 cells.

FIG. 30A-30B shows that inhibition of aldose reductase prevents growth factors-induced adhesion of HT29 cells to endothelial cells.

FIG. 31A-31C show that inhibition of aldose reductase prevents growth factors-induced expression of adhesion molecules in HT29 cells.

FIG. 32A-32B show that pharmacological inhibition of aldose reductase prevents metastatic tumor growth in nude mice.

FIG. 33A-33C show that siRNA ablation of aldose reductase prevents metastatic tumor growth in nude mice.

FIG. 34A-34D shows that inhibition of aldose reductase prevents expression of metastatic markers during colon cancer metastatic growth in nude mice liver.

FIG. 35A-35B show pharmacological inhibition of AR prevents metastatic tumor growth in nude mice.

FIG. 36A-36B show inhibition of AR preventing growth factors-induced invasion of HT29 cells (FIG. 36A) and inhibition of AR preventing growth factors-induced adhesion of HT29 cells to endothelial cells (FIG. 36B).

FIG. 37A-37B show that inhibition of AR prevents VEGF-induced tube (FIG. 37A) and spheroid (FIG. 37B) formation in HUVEC.

FIG. 38 shows inhibition of AR prevents VEGF-induced angiogenesis in matrigel plug model in rats.

### DETAILED DESCRIPTION OF THE INVENTION

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof. As used herein, the term “subject” refers to any target of the treatment.

The following abbreviations are used herein: AR: aldose reductase or human aldose reductase, ARL2, E.C. 1.1.1.21; sAR: Sus scrofa (Pig) aldose reductase, AR, E.C. 1.1.1.21; ARI: aldose reductase inhibitor; NADPH: dihydro-nicotinamide-adenine-dinucleotide phosphate; NADP: nicotinamide-adenine-dinucleotide phosphate; DCEG: S-(1,2-dicarboxyethyl)glutathione, γ-glutamyl-S-(1,2-dicarboxyethyl)cysteinylglycine; ROS: reactive oxygen species; CNS: Crystallography and NMR Software; GS or GSH: glutathione; γ-glutamylcysteinyiglycine; GS-HNE: glutathionyl-4-hydroxynonenal; GS-DHN: glutathionyl-1,4-dihydroxynonene; PGE2: prostaglandin E2; MTT: [3-(4,5-dimethylthiazol-2-yl)-5-(3-carboxymethoxyphenyl)-2-(4-sulfophenyl)-2H-tetrazolium salt]; BFGF: basic fibroblast growth factor; Cox: cyclooxygenase; DHN: 1,4-dihydroxynonene; HNE: 4-hydroxy-trans-2-nonenal; NF-κB: nuclear factor kappa binding protein; PKC: Protein kinase C; PDGF: platelet derived growth factor; SEAP: Secretory alkaline phosphatase; LPS:lipopolysaccharide; IKK: inhibitor of kappaB kinase; PLC: phospholipase C; iNOS: inducible NO synthase; VSMC: vascular smooth muscle cells; MAPK: mitogen activated protein kinase AOM: azoxymethane; ACF: aberrant crypt foci; and KO: Knockout.

Provided herein is a crystallized ternary complex of human aldose reductase bound to NADPH and γ-glutamyl-S-(1,2-dicarboxyethyl)cysteineinylglycine, a competitive inhibitor of AR-catalyzed reaction of glutathionyl-propanal (19). The ternary structure confirms the presence of two active sites within AR:NADPH. The crystal structure was determined to 1.9 Å and revealed novel interactions between the glutathione backbone and active site residues.

The ternary structure demonstrates that DCEG binding induces a significant conformational reorganization of the active site. The carboxylate moiety of DCEG binds in the aldose reductase active site, while the GS C-terminus binds in the aldose reductase loop C. The binding of glutathione to aldose reductase significantly reorients loops A and B of the protein thereby providing an induced-fit mechanism that enables the active site to bind substrates of different sizes. This induced-fit rearrangement and the multiplicity of specific interactions at the aldose reductase active site with glutathione are indicative of a highly selective glutathione-binding domain.

Thus, the ternary structure is used in methods of developing therapeutic inhibitors that selectively prevent binding of glutathione-conjugated substrates. These structure-based inhibitors are designed using rational drug design in conjunction with computer modeling of the coordinates of the ternary crystalline structure. The coordinates indicate that structure based inhibitors could be synthesized which will inhibit the glutathione-aldehyde binding site without affecting the detoxification role of aldose reductase since it will not inhibit the carbonyl binding site. For example, the specific inhibitors would not interfere the detoxification of free aldehydes, such as 4-hydroxy trans-2 nonenal which is formed during lipid peroxidation.

Also provided are the designed structure-based inhibitors and methods of screening therefor. The aldose reductase inhibitors may function through one of two mechanisms. Either remodeling of the aldose reductase loop-C backbone or steric hindrance of the GS-specific binding site in this loop may prevent the binding of GS-conjugates and their entry into the aldose reductase active site. A designed inhibitor may comprise a γ-glutamylcysteinylglycine backbone with an S-cysteinyl-substituted moiety that does not interfere with aldehyde binding to aldose reductase at the carbonyl active site.

These designed inhibitors may be tested for selective inhibition of glutathione-aldehyde binding in a screening assay. A selective inhibitor will form a complex with aldose reductase in the presence of NADPH by binding or otherwise interacting within the glutathione-binding domain in aldose reductase. Such a specific inhibitor will exclude glutathione-aldehyde binding and prevent subsequent reduction of the glutathione-aldehyde, but will not interfere with binding and reduction of the unconjugated lipid aldehyde at the carbonyl active site. Such screening assays are standard and well within the ordinary skill of an artisan to implement without undue experimentation or burden.

It is contemplated that other AKR proteins have similar sites that are capable of high affinity interactions with glutathione or glutathione conjugates. The same or similar techniques used to elucidate the AR:NADPH:DCEG ternary structure may be used to determine the coordinates of other similar AKR:ligand three-dimensional structures. Such crystal structures may be used in the design of relevant therapeutic inhibitors.

It is further contemplated that the aldose reductase inhibitors provided herein may be used as a therapeutic to treat or modulate or otherwise alter a pathophysiological state or event or symptoms thereof mediated by reduction products of aldose reductase as part of the pathology. For example, and without being limiting, a specific inhibitor could prevent glutathione binding without affecting the carbonyl reduction necessary to detoxify lipid aldehydes. Such inhibition could regulate TNF-α, growth factor, lipopolysaccharide, and hyperglycemia-induced cytotoxicity mediated by reactive oxygen species in, for example, the PLC/PKC/NF-κB pathway. It is further contemplated that such an inhibitor may limit access of other bulky molecules, such as glucose, to the aldose reductase active site thereby reducing other adverse effects of hyperglycemia as mediated by aldose reductase's role in the osmotic stress pathway.

Also provided are methods of inhibiting cell proliferation and/or metastasis of a cancer, e.g., colon cancer or lung cancer, with one or more of aldose reductase inhibitor compounds.

These aldose reductase inhibitor compounds are known in the art and can be easily synthesized by those skilled in the art using conventional methods of organic synthesis. These inhibitors are listed in Table 1. Other aldose reductase inhibitors will be known to those skilled in the art. Common chemical names or other designations are in parentheses where applicable.

TABLE 1
Aldose Reductase Inibitors

| | |
|---|---|
| 1. | 3,4-dihydro-4-oxo-3-[[5-(trifluoromethyl)-2-benzothiazolyl]methyl]-1-phthalazineacetic acid |
| | (zopolrestat); |
| 2. | (S)-6-fluorospiro[chroman-4,4′-imidazolidine]-2,5′-dione (sorbinil); |
| 3. | N-[(5-trifluoromethyl)-6-methoxy-1-naphthalenyl]thioxomethyl}-N-methylglycine (tolrestat); |
| 4. | 3-(4-bromo-2-fluorobenzyl)-3,4-dihydro-4-oxo-1-phthalazineacetic acid (ponalrestat); |
| 5. | 5-[(Z,E)-β-methylcinnamylidene]-4-oxo-2-thioxo-3-thiazolideneacetic acid (epalrestat); |
| 6. | 3-(4-bromo-2-fluorobenzyl)-7-chloro-3,4-dihydro-2,4-dioxo-1(2H) quinazoline acetic acid |
| | (zenarestat); |
| 7. | 3,4-dihydro-3-oxo-4-[(4,5,7-trifluoro-2-benzothiazolyl)methyl]-2H-1,4-benzothiazine-2-acetic |
| | acid (SPR-210); |
| 8. | N-[3,5-dimethyl-4-[(nitromethyl)sulfonyl]phenyl]-2-methylbenzeneacetamide (ZD5522); |
| 9. | (2S,4S)-6-fluoro-2′,5′-dioxospiro(chroman-4,4′-imidazolidine)-2-carboxamide (fidarestat); |
| 10. | 2-[(4-bromo-2-fluorophenyl)methyl]-6-fluorospiro[isoquinoline-4(1H),3′-pyrrolidine]- |
| | 1,2′,3,5′(2H)-tetrone (minalrestat); |
| 11. | 2R,4R-6,7-dichloro-4-hydroxy-2-methylchroman-4-acetic acid; |
| 12. | 2R,4R-6,7-dichloro-6-fluoro-4-hydroxy-2-methylchroman-4-acetic acid; |
| 13. | 3,4-dihydro-2,8-diisopropyl-3-oxo-2H-1,4-benzoxazine-4-acetic acid |
| 14. | d-2-methyl-6-fluoro-spiro(chroman-4′,4′-imidazolidine)-2′,5′-dione; |
| 15. | 2-fluoro-spiro(9H-fluorene-9,4′-imidazolidine)-2′,5′-dione; |
| 16. | 2,7-di-fluoro-spiro(9H-fluorene-9,4′-imidazolidine)-2′,5′-dione; |
| 17. | 2,7-di-fluoro-5-methoxy-spiro(9H-fluorene-9,4′-imidazolidine)-2′,5′-dione; |
| 18. | 7-fluoro-spiro(5H-indenol[1,2-b]pyridine-5,3′-pyrrolidine)-2,5′-dione; |
| 19. | d-cis-6′-chloro-2′,3′-dihydro-2′-methyl-spiro-(imidazolidine-4,4′-4′H-pyrano(2,3-b)pyridine)- |
| | 2,5-dione; |
| 20. | spiro[imidazolidine-4,5′(6H)-quinoline]-2,5-dione-3′-chloro-7,′8′-dihydro-7′-methyl-(5′-cis); |
| 21. | 3,4-dihydro-3-(5-fluorobenzothiazol-2-ylmethyl)-4-oxophthalazin-1-yl-acetic acid; |
| 22. | 3-(5,7-difluorobenzothiazol-2-ylmethyl)-3,4-dihydro-4-oxophthalazin-1-yl-acetic acid; |
| 23. | 3-(5-chlorobenzothiazol-2-ylmethyl)-3,4-dihydro-4-oxophthalazin-1-yl-acetic acid; |
| 24. | 3-(5,7-dichlorobenzothiazol-2-ylmethyl)-3,4-dihydro-4-oxophthalazin-1-yl-acetic acid; |
| 25. | 3,4-dihydro-4-oxo-3-(5-trifluoromethylbenzoxazol-2-ylmethyl)phthalazin-1-yl-acetic acid; |
| 26. | 3,4-dihydro-3-(5-fluorobenzoxazol-2-ylmethyl)-4-oxophthalazin-1-yl-acetic acid; |
| 27. | 3-(5,7-difluorobenzoxazol-2-ylmethyl)-3,4-dihydro-4-oxophthalazin-1-yl-acetic acid; |
| 28. | 3-(5-chlorobenzoxazol-2-ylmethyl)-3,4-dihydro-4-oxophthalazin-1-y-lacetic acid; |
| 29. | 3-(5,7-dichlorobenzoxazol-2-ylmethyl)-3,4-dihydro-4-oxophthalazin-1-yl-acetic acid. |

Alternatively, the present invention provides methods of inhibiting expression of aldose reductase at the RNA translational level. It is contemplated that administration of aldose reductase small interfering RNAs (siRNA) is useful in the treatment of a pathophysiological state, such as a cancer, for example, but not limited to, colon cancer, lung cancer or a metastatic cancer derived therefrom. The siRNAs may be useful in the treatment of or alleviation of other pathophysiological conditions or symptoms resulting from aldose reductase-mediated signaling of a cytotoxic pathway. For example, conditions exhibiting or characterised by inflammation, e.g., lipopolysaccharide-induced inflammation, may benefit from such treatment or therapy. In addition it is contemplated that the aldose reductase inhibitors described herein also may inhibit metastasis of a cancer cell to form a metastatic cancer, for example, but not limited to, the metastasis of a colon cancer, such as a colorectal cancer, to the liver. Contacting a cancer cell with one or more of these inhibitors is effective to prevent migration of a cancer cell through an extracellular matrix. As is apparent to one of ordinary skill in the art contact includes any known method effective to provide the aldose reductase inhibitor to the cell.

The design methodology for siRNAs is known in the art and/or they may be obtained commercially. For example, without being limiting, an siRNA effective as a therapeutic may have the sequence of SEQ ID NO: 1. siRNAs may be administered to a subject as the naked oligomer or as comprising a suitable transfection vector or with a carrier molecule or moiety as are known and standard in the art.

It is standard in the art to formulate a therapeutic compound with a pharmaceutically acceptable carrier as a pharmaceutical composition. It is standard in the art to determine dose, dosage and routes of administration of the therapeutic or pharmaceutical compounds. Such determination is routinely made by one of skill in the art based on the individual and the particular pathophysiological state or symptoms exhibited by the patient and the patients history. The following examples are given for the purpose of illustrating embodiments of the invention and are not meant to limit the present invention in any fashion.

### EXAMPLE 1

### Aldose Reductase Crystallography and Inhibitor Design

### Overexpression and Purification of Recombinant Human Aldose Reductase

Recombinant human aldose reductase was over expressed and purified as described (23). In brief, the cell extract was subjected to chromatofocusing on PBE94 (Pharmacia LKB Biotechnology Inc.) followed by hydroxylapatite column chromatography and reactive blue affinity chromatography as the final step. All purification buffers contained 1 mM dithiothretiol (DTT).

### Crystallization of the Ternary Complex

Purified aldose reductase was concentrated by ultrafiltration (Amicon YM-10 membrane) to ˜10 mg/ml. Prior to crystallization, 10 mg/ml aldose reductase in phosphate buffer (10 mM phosphate pH 7.1, 0.5 mM EDTA, 10 mM DTT) was incubated with NADPH and DCEG (γ-glutamyl-S-(1,2-dicarboxyethyl)glutathione) at a AR:NADPH:DCEG molar ratio of 1:2:2 for 10 min at 4° C. The ternary complex was crystallized using the vapor diffusion method at 4° C. The protein:ligand solution was mixed with an equal volume of 22% (w/v) polyethylene glycol (PEG) 4000 in 100 mM sodium citrate (pH 5.0) and 6 ml of droplets were placed above an identical well solution.

### Data Collection

X-ray data were collected using a MacScience DIP 2030H area detector and a M06XHF rotating anode X-ray generator operating at 50 KV and 90 Ma and equipped with Göbel collimating optics (Bruker AXS). The first crystal, 0.1×0.1×0.1 mm³, was flash-cooled, without the addition of cryo-protectants to the drop, using nitrogen boil-off (Cryo Industries). Weak ice rings were observed in the diffraction pattern. The protein crystallized in the P2₁ monoclinic space group with cell dimensions a=47.21 Å, b=66.72 Å, c=49.30 Å, a=g=90.00°, b=92.24°. This crystal form was not observed previously for any aldose reductase crystal structures. Based upon the Matthews coefficient (24), there was predicted to be one aldose reductase molecule per au. The data were processed to 2.6 Å resolution using the programs HKL (25). A second crystal was soaked in mother liquor containing 20% glycerol (v/v) and 25 mM of DCEG and flash cooled. Diffraction data collected from crystal 2 were processed with HKL to 1.94 Å resolution and was used for high-resolution refinements of the model. Space group and unit cell dimensions were similar to crystal 1. Data collection and processing statistics, including atomic coordinates and structure factors, for crystal 2, i.e., 1Q9N, are shown in Table 2.

TABLE 2
Summary of crystallographic statistics
PDB Accession ID

| | P2₁ |
|---|---|
| Space group | |
| Cell | |
| a (Å) | 47.21 |
| b (Å) | 66.72 |
| c (Å) | 49.30 |
| a (°) | 90.00 |
| b (°) | 92.24 |
| g (°) | 90.00 |
| Data Collection | |
| Resolution range, Å | 30-1.94 |
| R_{merge},* % | 9.0 (30.7) |
| Unique observations | 22,256 |
| Average I/s (I)* | 13.8 (4.4) |
| Redundancy* | 5.8 (3.2) |
| Completeness* % | 97.7 (87.8) |
| Refinement Statistics | |
| R-factor (%)* | 21.1 (26.4) |
| R_{free},* % | 26.0 (34.9) |
| r.m.s. deviations | |
| Bonds (Å) | 0.006 |
| Angles (°) | 1.3 |
| Model Statistics | |
| No. residues in most favored region | 249 |
| Additional allowed | 25 |
| Generously allowed | 3 |
| Disallowed | 0 |
| No. Protein Atoms | 2517 |
| No. Ligand atoms | 76 |
| No. Waters | 165 |
| Average B factor (Å²) | 20.4 |
| Protein (Å²) | 19.4 |
| Waters (Å²) | 25.1 |

*Values for the highest-resolution shells are in parentheses.

### Structure Determination and Refinement

The P2₁ crystal form structure was solved by molecular replacement using the program EPMR (26) with the 1ADS (3) structure as a search model. Initial model building in CNS (27) used data collected to 2.6 Å resolution from crystal 1. Since this data set contained scattering noise from ice crystals, the initial refinement contained resolutions shells with unusually high R-factors. An alternate processing of this data, which removed all reflections in the narrow resolution range affected by the ice, also was used for model building.

The PMB suite of programs (28) was used to generate a test set using 5% of the reflections chosen in thin shells equally spaced in 1/d. The PMB suite was used as an interface to the structure refinement program CNS to simplify and partially automate the structure refinement process. The variable sigma model of B-factor restraints (29) was implemented in CNS and the parameters optimized to minimize the free R. This led to a significant reduction in the free R value. The result was a model that had the least bias without over-fitting free parameters (30,31).

An initial rigid body refinement was followed by repetitive rounds of isotropic variable sigma B-factor and positional refinement, until the free R factor (32) no longer decreased (The

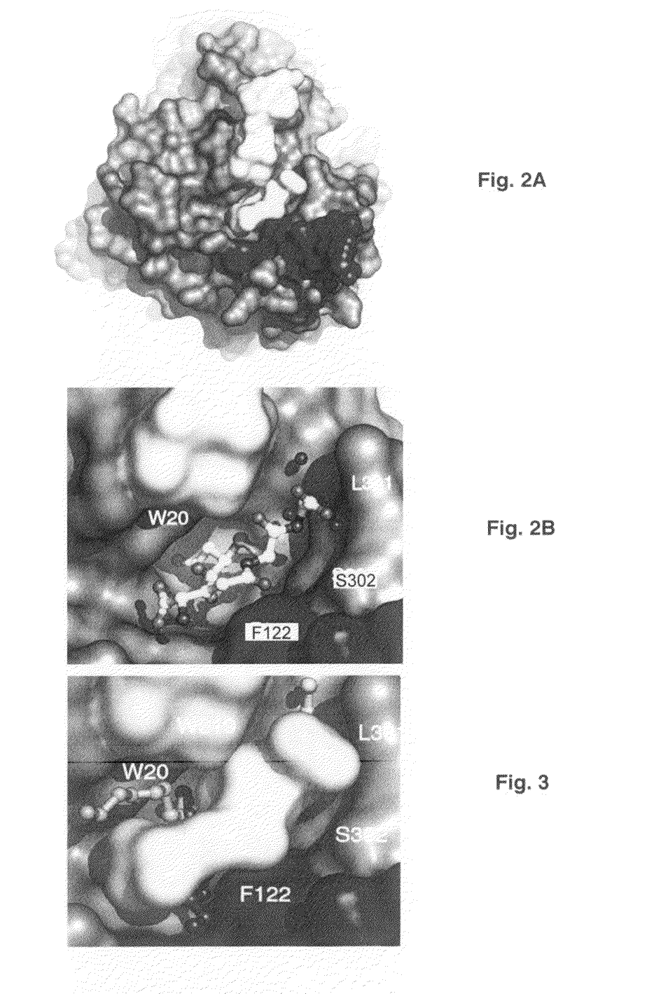

PMB software suite is available from the author M.A.W. (www.xray.utmb.edu/PMB)). The model was rebuilt in iterative rounds of model building (Xtalview (33)) and refinement. Structure factors were corrected for anisotropic scattering and absorption using a local scaling algorithm (28,34,35). The DCEG (FIG. 2A) inhibitor was modeled using Insight II (Accelrys, San Diego, Calif.) and energy minimized using the PRODRG web server (36), which also generated the stereochemical restraints used in the structure refinement.

The second P2₁ crystal structure was solved using the partially refined 2.6 Å model. The initial rigid body refinement was followed by repetitive rounds of individual atomic isotropic variable sigma B-factor and positional refinement, until the free R factor no longer decreased. Model building included the examination of waters selected by CNS. Waters with excessive B-factors (>60 Å²) or poor density correlation were deleted.

Model quality was assessed after each refinement step with XtalView or PROCHECK (37). Refinement of the final model proceeded in parallel with alternate conformations of the DCEG ligand. The model with the lowest free R was chosen as the final model. The DCEG ligand of this model produced the best fit to the electron density from the two separate refinements. Multiple conformation refinement of DCEG in REFMAC (38, 39), including TLS anisotropic B-factors, with a single aldose reductase model and the two DCEG models confirmed that the chosen conformation had the highest correlation with the observations. All molecular figures were generated using PYMOL (40).

### Overall Structure

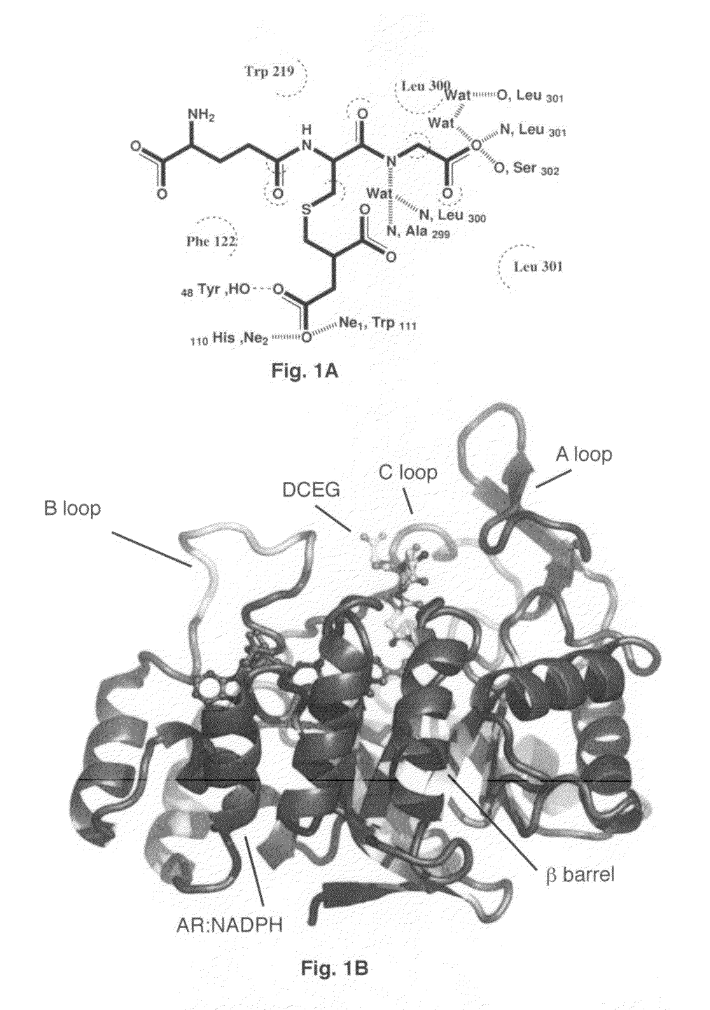

The AR:NADPH:DCEG ternary complex structure was refined to 1.94 Å resolution with a final R-factor of 21.6%. This structure showed well-defined electron density for the DCEG substrate at the “top” of aldose reductase active site pocket (FIG. 2B). The DCEG was bound between two opposing surfaces of the active site pocket, but did not completely fill the active site cleft (FIG. 1A). The DCEG substrate made ˜80 contacts, defined as inter-residue distances≦4 Å, with residues in the active site cleft (FIG. 1A). The majority of these intermolecular contacts were hydrophobic. The NADPH binding site was located at the base of the aldose reductase hydrophobic active site pocket and the NADPH cofactor was bound to the ternary complex in an orientation identical to that observed in previously reported crystal structures (3, 41, 42).

The active site of aldose reductase sat at the base of a deep cleft or binding pocket. The sides of the active site pocket were formed by three flexible loops A, B, and C (43) which sat on top of the aldose reductase (α/β)₈ barrel (FIG. 1B). The active site comprises residues Tyr-48, His-110, and Trp-111. DCEG was bound in the active site almost filling the active site pocket. Trp-219 forms one side of the narrow pocket holding the inhibitor DCEG (FIG. 2B). The other residues lining this pocket included Trp-20, Trp-79, Trp-111, Phe-122, NADPH, Val-47, Cys-298, Ala-299, Leu-300, and Leu-301.

DCEG Interactions with Aldose Reductase

The C-terminal glycine moiety of DCEG was extensively hydrogen bonded to the backbone atoms of residues 300-302 in the flexible human aldose reductase C-terminal loop (loop-C). In addition, the ligand made several van der Waals contacts with aldose reductase. Several bound water molecules mediated the interaction between the DCEG glycine moiety and aldose reductase. The amides of Ala-299 and Leu-300 were bound indirectly to DCEG through a water molecule. The terminal carboxylate group of the DCEG interacted with the backbone of Leu-301 and Ser-302 and indirectly with Leu-301 through a network of waters (FIG. 2A). These residues were in human aldose reductase loop C which has been shown to be important for enzymatic activity. Mutations within this loop result in drastically lowered human aldose reductase activity (44).

The dicarboxyethyl group of DCEG was anchored in the conserved anion-binding site between the nicotinamide ring of the NADPH cofactor and aldose reductase residues Tyr-48, His-110, and Trp-111 similar to other known aldose reductase inhibitors (41,42). The terminal carboxylates of the dicarboxyethyl conjugate's longer arm, Oi2 and Oj2, were hydrogen bonded to active site residues His-110, Tyr-48, and Trp-111 (FIG. 1A, 2B). The γ-glutamate of DCEG was observed to interact with the AR enzyme only through van der Waals contacts with Phe-122 that formed one side of the hydrophobic active site pocket. The lack of hydrogen bonds or extensive contacts permitted the γ-glutamate moiety significant conformational freedom.

The higher temperature factors for these atoms reflected the relative disorder in the N-terminal end of DCEG. The hydrophobic walls of the upper portion of the aldose reductase active site pocket were formed in large part by Trp-219 and Phe-122, similar to the structures observed in other aldose reductase:inhibitor complexes (41,42). These two aromatic residues tightly constrained the position of the cysteine moiety in DCEG. The Phe-122 and Trp-219 side chains could move slightly to accommodate differently sized inhibitors. The extensive van der Waals contacts with Trp-20 observed in the aromatic inhibitors toirestat, zopolrestat, and sorbinil were completely absent in DCEG. The Trp-20 and Trp-79 residues, although still defining the active site pocket, did not interact with DCEG directly. They did, however, limit the conformational space available to the DCEG molecule.

The conformation of the glutathione (GS)-moiety of the aldose reductase-bound DCEG (FIG. 2B) was similar to the conformation of GS observed in the GS-binding proteins glutathione-S-transferase (45), sphingomonad GST (1fe2 (46)), human thioltransferase (47), yeast prion URE2P (48), and the chloride intercellular channel (49). The GS backbone conformation of DCEG was most distinct from the conformation of GS bound to glutatione reductase (1b4q (47), 1gra (50)). The GS conformation of aldose reductase-bound DCEG adopted the low energy Y-shape, rather than the V-form of GS observed in glutaredoxin (47), glutathione reductase (47, 50), and glutathione peroxidase (51,52) complexes.

The GS backbone of DCEG overlapped with the GS structures with root mean square deviations (rmsd) from 0.4 to 1.4 Å. The largest rmsd between the observed structures of GS bound to several different enzymes and DCEG bound to aldose reductase occurred in the N- and C-terminal atoms. In comparison with GS bound to glutathione reductase, the cysteine of DCEG bound to aldose reductase had a y angle that was rotated by ˜180 degrees. The aldose reductase-bound DCEG glutathione backbone conformation was most similar to that observed in GS complexes with hematopoietic prostaglandin d synthase (53) or yeast prion URE2P (48).

DCEG binding to aldose reductase lacks the N-terminal hydrogen bonds seen in the other GS:protein complexes. The placement of the GS backbone was largely determined by the interaction of the conjugate with the active site of the enzyme and the mobile loop-C. The van der Waals interactions with the binding cleft were nonspecific and allowed for flexibility of the GS moiety.

Comparison with Other Aldose Reductase Structures

The structure of the human aldose reductase enzyme within the ternary complex showed significant conformational differences relative to the AR:NADPH binary complex (3). The backbone atoms of Pro-123 to Val-131 in loop A and Pro-218 to Pro-225 in loop B, which flank the active site pocket, were reoriented >5 Å upon DCEG binding relative to the binary structure. The AR:NADPH:DCEG ternary complex more closely resembled the AR:NADP:zopolrestat (54) and AR:NADP:Idd384 (41) ternary complexes than the AR:NADPH binary complex. In the ternary complexes the largest relative atomic movements, with rmsd>1 Å, occurred in the region of Ser-127, Pro-222, and Leu-300.

The conformation of loop B, residues Pro-218 to Pro-225, was very similar in all of the aldose reductase structures, with just the backbone conformation of residues Pro-222 and Asp-224 flipping in the holoenzyme. Loop A of the holoenzyme structure (3) displayed a completely different conformation for this entire loop region relative to the current complex. Loop C was observed in two different conformations, which depended on the size and shape of the inhibitor bound in the solved aldose reductase structures. The conformation of loop C in AR:NADPH:DCEG had the greatest similarity to the human aldose reductase structures found in the AR:NADPH holoenzyme (3) and AR:NADPH:Idd384 ternary complex (41). Additionally, loop C in the current structure had large positional differences with the conformation observed in the zoplorestat and tolrestat ternary complexes (42). This indicated that loop C was dynamic and could move to accommodate larger molecules such as zopolrestat and tolrestat. The smaller sorbinil inhibitor did not change this loop's conformation significantly (42).

Comparison with Molecular Dynamics Models

Based on molecular dynamics (MD) simulations on a GS-propanal conjugate binding to human aldose reductase (19), two possible alternate conformations of the bound substrate were proposed. The observed structure of DCEG in the AR:NADPH:DCEG ternary complex was very similar to the first, lowest energy model (Model 1) of our molecular dynamics simulation, i.e., 0.8 Å overall rmsd on the GS-backbone and 0.5 Å rmsd, excluding the disordered N-terminus of the substrate. The small variations between the model and DCEG structure could be attributed to the change in the active-site atoms from carbonyl in GS-propanal to a carboxylate in DCEG, and the conformational freedom of the γ-glu N-terminus.

It has been demonstrated that DCEG is a competitive inhibitor of aldehyde reduction by aldose reductase, indicating that the conjugate bound selectively to AR:NADPH and had little or no affinity for the enzyme of the AR:NADP⁺ binary complex. The reasons for this behavior are apparent from the current structure. The non-specific interactions of DCEG with the active site cleft and loose shape complimentarity are consistent with a very low affinity of DCEG for apo aldose reductase.

The result of NADPH binding is rearrangement of the active site residues Tyr-48, His-110 and Trp-111, plus the adjacent A, B, and C loops. Thus, NADPH binding reorients these regions to form the active site pocket. It is only after these rearrangements that aldose reductase would have any significant affinity for DCEG. Therefore, DCEG binding must be preceded by formation of the holoenzyme AR:NADPH complex.

In the AR:NADPH:DCEG ternary complex, a larger percentage, i.e., 50%, of DCEG is buried by aldose reductase side chains than has been observed in structures of other GS-binding proteins (40-45%), suggesting that the strongly aliphatic nature of DCEG, which allows multiple contacts at the active site, was essential for competitive inhibition of aldehyde reduction. This was due to selective binding to the AR:NADPH binary complex. In contrast, more aromatic inhibitors, which bind to the aldose reductase active site primarily via hydrophobic interactions, bind with greater affinity to the AR:NADP+ binary complex and thus behave as non-competitive inhibitors of aldehyde reduction, but competitive inhibitors of alcohol oxidation (19).

### DCEG-Based Inhibitor Design

The structure of DCEG bound to aldose reductase provides a starting model for the design of an inhibitor of aldose reductase carbonyl metabolism which would not significantly interfere with aldose reductase detoxification of reactive aldehydes. The proposed GS-based inhibitor binding in the DCEG site would permit long alkyl chain peptides to reach the active site. Modeling of a DCEG-like selective inhibitor, based on our AR:NADPH:DCEG structure with an alkyl chain bound in the active site showed that there was more than one possible path for the alkyl chain to reach the active site (FIG. 3). Therefore, a DCEG-like inhibitor, lacking the active-site binding dicarboxyethyl moiety, could potentially block the binding of glucose and GS-conjugates while still permitting the entry and reduction of small to medium chain aliphatic aldehydes. By using such inhibitors, it might be possible to prevent the reduction of glucose to sorbitol in diabetics, conserve NADPH that can be used for the reduction of lipid peroxides and aldehydes, and regulate signaling pathways initiated by cytokines, chemokines, hyperglycemia, etc. without affecting the detoxification properties of aldose reductase that may be essential for reducing lipid aldehydes. Thus, a DCEG-based inhibitor might provide a therapeutic tool for regulating cytotoxic signals without inhibiting the detoxification role of aldose reductase.

### EXAMPLE 2

### Aldose Reductase Inhibition

McCoy's 5A medium, Dulbecco's modified Eagle's medium (DMEM), phosphate-buffered saline (PBS), penicillin/streptomycin solution, trypsin, and fetal bovine serum (FBS) were purchased from Invitrogen. Antibodies against Cox-1, Cox-2 and phospho PKC-b2 were obtained from Santa Cruz Biotechnology, Inc. (Santa Cruz, Calif.). Sorbinil and tolrestat were gifts from Pfizer and American Home Products, respectively. Mouse anti-rabbit glyceraldehyde-3-phosphate dehydrogenase antibodies were obtained from Research Diagnostics Inc.

Cyclooxygenase (Cox) activity assay and prostaglandin E2 (PGE2) assay kits were obtained from Cayman Chemical Company (Ann Arbor, Mich.). Platelet-derived growth factor (PDGF), basic fibroblast growth factor (BFGF), 3-(4,5-dimethylthiazol-2-yl)-2,5-diphenyltetrazolium bromide (MTT), and other reagents used in the Electrophoretic Mobility Shift Assay (EMSA) and Western blot analysis were obtained from Sigma. AR-siRNA (5′-AATCGGTGTCTCCAACTTCAA-3′; SEQ ID NO: 1) or scrambled siRNA (control) (5′-AAAATCTCCCTAAATCATACA-3′; SEQ ID NO: 2) were synthesized by Dharmacon Reseacrh. All other reagents used were of analytical grade.

### Cell Culture

Human colon cancer cell lines, HCT-116 and Caco-2 were obtained from American type culture collection (ATCC). HCT-116 cells were maintained and grown in McCoy's 5A medium supplemented with 10% FBS and 1% penicillin/streptomycin and Caco-2 cells were grown in DMEM with 10% FBS and 1% penicillin/streptomycin at 37° C. in a humidified atmosphere of 5% CO₂. Human colon adenocarcinoma (SW480) cells were purchased from ATCC and cultured at 37° C. in a humidified atmosphere of 5% CO₂ in RPMI-1640 medium supplemented with 10% (v/v) heat-inactivated FBS, 1% (v/v) P/S solution, 2 mM L-glutamine, 10 mM HEPES, 1 mM sodium pyruvate, 4.5 g/L glucose, and 1.5 g/L sodium bicarbonate.

### Measurement of Cytotoxicity

Caco-2 cells were grown to confluence in DMEM medium harvested by trypsinization and plated ˜2500 cells/well in a 96-well plate. Subconfluent cells were growth-arrested in 0.1% FBS. After 24 h, 10 ng/ml of BFGF or PDGF without or with aldose reductase inhibitors sorbinil or tolrestat were added to the media and the cells were incubated for another 24 h. Cells incubated with the aldose reductase inhibitors alone served as control. Cell viability was determined by cell count and MTT-assay as described earlier (15, 55-56).

HT29 cells were grown to confluence in McCoy's medium and harvested and plated 2,500 per well in a 96-well plate. Subconfluent cells were growth arrested in 0.1% FBS with or without aldose reductase inhibitor sorbinil or zopolrestat (20 μM). After 24 hours, EGF (5 ng/ml) or BFGF (10 ng/ml) was added to the medium and the cells were incubated for another 24 hours. Cells incubated with the aldose reductase inhibitors alone served as control. Cell viability was determined by an MTT assay.

A549 cells were grown to confluence in Ham's F12K medium containing 10% FBS and harvested, and plated at 5000 cells per well in a 96-well plate. After 24 hrs A549 cells were treated with or without aldose reductase inhibitor sorbinil or tolrestat with various concentrations for another 24 hrs. Cells incubated without aldose reductase inhibitors served as control. Cell viability was determined by and MTT assay.

### Determination of PKC Activity

PKC activity was measured using the Promega-Sigma TECT PKC assay system as described (15). Briefly, aliquots of the reaction mixture (25 mM Tris-Hcl pH 7.5, 1.6 mg/mL phosphatidylserine, 0.16 mg/mL diacylglycerol, and 50 mM MgCl₂) were mixed with [γ-³²P] ATP (3,000 Ci/mmol, 10 μCi/μL) and incubated at 30° C. for 10 min. To stop the reaction, 7.5 M guanidine hydrochloride were added and the phosphorylated peptide was separated on binding paper. The extent of phosphorylation was detected by measuring radioactivity retained on the paper.

### PGE2 Assay

Caco-2 cells were plated in 6 well plates at a density of 2×10⁵ cells/well. After 24 hours, the medium was replaced with fresh medium containing 0.1% serum with or without, sorbinil (20 μM) followed by treatment with either 10 ng/ml BFGF or PDGF, for another 24 h. HT29 cells were plated in 6 well plate at a density of 2×10⁵ cells/well. After 24 hours, medium was replaced with serum-free medium with or without, sorbinil or zopolrestat (20 μM) followed by treatment with either EGF (5 ng/ml) or BFGF (10 ng/ml) for another 24 h. The medium from each cell lines was collected from each well and analyzed for PGE2 by using an Enzyme Immuno Assay kit according to the manufacturer's instructions (Cayman Chemical Co., Inc.).

Briefly, 50 μl of diluted standard/sample were pipetted into a pre-coated goat polyclonal anti-mouse IgG 96-well plate. Aliquots (50 μl) of a PGE2 monoclonal antibody and PGE2 acetylcholine esterase (AchE) conjugate, (PGE2 tracer) were added to each well and allowed to incubate at 4° C. for 24 h. After incubation, the wells were washed five times with wash buffer containing 0.05% Tween-20, followed by the addition of 200 μl of Ellman's reagent containing acetylthiocholine and 5,5′-dithio-bis-(2-nitrobenzoic acid). Samples were read after 60 min at 412 nm with an ELISA reader. In this procedure the intensity of yellow color, is proportional to the amount of PGE2 tracer bound to the well and is inversely proportional to the amount of free PGE2 present in the well during incubation.

### Cyclooxygenase Activity Assay

For determination of Cox activity growth-arrested Caco-2 cells were treated with either 10 ng/ml BFGF or PDGF in the absence and presence of sorbinil (20 mM) for 24 h. The cells were harvested and homogenized in cold (4° C.) buffer containing 0.1M Tris-Hcl, pH 7.8 and 1 mM EDTA and the activity was measured in 96 well plate according to the manufacturer's (Cayman Chemical Co., Inc.) instructions. Briefly, 10 μl of standard/sample were incubated in the presence of arachidonic acid and substrate, N,N,N,N-tetra methyl-p-phenylenediamine (TMPD) in a total reaction volume of 210 μl. The Cox peroxidase activity was measured colorimetrically by monitoring appearance of oxidized TMPD at 590 nm by using ELISA reader.

### NF-kB-Dependent Reporter Secretory Alkaline Phosphatase (SEAP) Expression Assay.

Caco-2 cells (1.5×10⁵ cells/well) were plated in six-well plates and after attachment overnight, were serum-starved in optiMEM medium for 24 h with or without aldose reductase inhibitor, sorbinil (20 μM) and were transiently transfected with pNF-kB-SEAP construct or control plasmid pTALSEAP DNA (Clontech, USA) using the lipofectamine plus reagent. After 6 h of transfection, cells were treated either with 10 ng/ml BFGF or PDGF for 48 h in DMEM medium containing 0.1% FBS. The cell culture medium was then harvested and analyzed for SEAP activity, essentially as described by the manufacturer (Clontech Laboratories, Palo Alto, Calif.), using a 96-well chemiluminiscence plate reader and Kodak Image Station 2000R.

### Determination of NF-kB Activation

The cytosolic as well as nuclear extracts were prepared as described earlier (15) and the NF-kB activity was determined by using the colorimetric non-radioactive NF-kB p65 Transcription Factor Assay kit (Chemicon Intl.) as per the supplier's instructions. Briefly, a double stranded biotinylated oligonucleotide containing the consensus sequence for NF-kB binding (5′-GGGACTTTCC-3′; SEQ ID NO: 3) was mixed with nuclear extract and assay buffer. After incubation, the mixture (probe+extract+buffer) was transferred to the streptavidin-coated ELISA kit and read at 450 nm using an ELISA plate reader. For each experiment, triplicate samples were measured for statistical significance.

### RT-PCR

Total RNA was isolated from Caco-2 cells by using Rnaeasy micro isolation kit (Qiagen). Total RNA (1.5 μg) sample was reverse transcribed with Omniscript and Sensiscript reverse transcriptase one-Step RT PCR system with HotStarTaq DNA polymerase (Qiagen) at 55° C. for 30 min followed by PCR amplification. The oligonucleotide primer sequences were as follows: 5′-AAACCCACTCCAAACACAG-3′ (sense; SEQ ID NO: 4) and 5′-TCATCAGGCACAGGAGGAAG-3′ (antisense; SEQ ID NO: 5) for Cox-2, and 5′-TGAGACCTTCAACACCCCAG-3′ (SEQ ID NO: 6) and 5′-TTCATGAGGTAGTCTGTCAGGTCC-3′ (SEQ ID NO: 7) for β-actin. PCR reaction was carried out in a GeneAmp 2700 thermocycler (Applied Biosystems, Foster City, Calif.) under the following conditions: initial denaturation at 95° C. for 15 min; 35 cycles of 94° C. 30 s, 62° C. 30 s, 72° C. 1 min, and then 72° C. 5 min for final extension (57). PCR products were electrophoresed in 2% Agarose-1™ TAE gels containing 0.5 μg/ml ethidium bromide.

### Flowcytometric Analysis of Cell Cycle

The Caco-2 and A549 cells were grown separately in 6 well plates at a density of approximately 1.5×10⁵ cells/well. Growth-arrested Caco-2 cells were pre-incubated with or without sorbinil 20 μM or carrier for 24 h and then stimulated with either 10 ng/ml BFGF or PDGF for another 24 h. After 24 hrs A549 cells were incubated with or without sorbinil at different concentrations for another 24 h.

The Caco-2 and A549 cells were then washed with PBS and harvested by trypsinization. Cellular DNA was stained with low and high salt solutions. Briefly, cells were resuspended in 250 μl of solution A, low salt stain, containing polyetheleneglycol (30 mg/ml), propidium iodide (0.05 mg/ml), triton-x-100 (1 μl/ml), sodium citrate 4 mM, RNAse A 10 μg/ml and incubated at 37° C. for 20 min followed by the addition of 250 μl of solution B, high salt stain containing 400 mM NaCl instead of 4 mM sodium citrate in solution A, and incubated overnight at 4° C. Cell cycle analysis was performed with a minimum of 10,000 events per analysis by using FACScan flow cytometer (Becton, Dickinson and Co., San Jose, Calif., USA).

### Measurement of Reactive Oxygen Species

Caco-2 cells were plated in a 24-well plate at a density of 1.5×10⁴ cells/well in DMEM and then serum-starved at 60-70% confluence in the absence and presence of 20 μM sorbinil or tolrestat for overnight in phenol red-free DMEM supplemented with 0.1% FBS. Cells were then pre-incubated for 30 min with the ROS-sensitive fluorophore 2′,7′-dichlorofluorescein diacetate (DCFH-DA), which is taken up and oxidized to the fluorescent dichlorofluorescein by intracellular ROS. After incubation with DCFH-DA, the cells were exposed to FGF or PDGF 10 ng/ml for 60 min and fluorescence was measured with a CytoFluorll fluorescence plate reader (PerSeptive Biosystems, Inc., Framingham, Mass.) at excitation of 485 nm and emission of 528 nm.

The levels of ROS in sections of A549 xenografts was determined using dihydroethidium (Het; Molecular Probes, Eugene, Oreg.). The Het dye gives red fluorescence when oxidized to EtBr in the presence of ROS such as O₂⁻. Serial sections (5 μM) of para-formaldehyde fixed xenografts were deparafinized and rehydrated and incubated with Het dye (5 μM in PBS) for 30 min at 37° C. followed by acquisition of images using a fluorescence microscope 200× magnification.

### Preparation of GS-Aldehyde Esters

HNE was synthesized as described (14). The glutathione monoethyl-ester (GS-ester) obtained from Sigma was purified by HPLC using a reverse phase column (14) and the conjugate of GS-ester and HNE was made by incubating 1 μmol of [4-³H]-HNE with 3-fold excess of GS-ester and 0.1 M potassium phosphate, pH 7.0, at 37° C. The reaction was followed by monitoring absorbance at 224 nm. Approximately 90% of HNE was conjugated with GSH over a period of 60 min. The GS-HNE-ester thus formed was purified by HPLC (14) and its concentration was calculated on the basis of radioactivity. For synthesis of GS-DHN-ester, 1 μmol of GS-HNE-ester was incubated with 1 unit of recombinant human aldose reductase and 0.1 mM NADPH in 0.1 M potassium phosphate, pH 7.0, at 37° C. The reaction was followed by monitoring the decrease in absorbance at 340 nm. More than 85% of the conjugate was reduced in 30 min. The enzyme was removed by ultrafiltration using an Amicon Centriprep-10, and GS-DHN-ester in the filtrate was purified on HPLC and confirmed by ESI/MS.

### Antisense Ablation of Aldose Reductase

Caco-2 cells were grown to 50-60% confluence in DMEM supplemented with 10% FBS and washed four times with Opti-MEM, 60 min before the transfection with oligonucleotides (15). The cells were incubated with 2 μM aldose reductase antisense or scrambled control oligonucleotides using LipofectAMINE Plus (15 μg/ml) as the transfection reagent as suggested by the supplier. After 12 h, the medium was replaced with fresh DMEM (containing 10% FBS) for another 12 h followed by 24 h of incubation in serum-free DMEM (0.1% FBS) before growth factor stimulation. Changes in the expression of aldose reductase were estimated by Western blot analysis using anti-AR antibodies.

### Cell Invasion Assay

HT29 cells were serum starved in McCoy's medium with or without sorbinil for 24 hrs. HT29 cells and A549 cells each were plated 0.8×10⁵ cells per well in a 96 well plate containing culture inserts of an 8.0-μm polycarbonate membrane which is coated with a thin layer of ECMatrix. HT29 cells then were treated with 5% FBS or EGF (5 ng/ml) with or without sorbinil or zopolrestat (75 μM). A549 cells then were treated with 5% FBS in Ham's F12K medium with or without sorbinil or zopolrestat (100 μM). Both HT29 and A549 plates were transferred to a feeder tray with McCoy's medium (HT29) or Ham's F12K medium (A549) each containing 5% FBS. The HT29 and A549 cells were incubated at 37° C. under a 5% CO₂ atmosphere. After 24 hrs invaded cells at bottom of the culture inserts were rinsed with PBS and incubated for in a detachment solution for 30 min at 37° C. Then cells were incubated in lysis buffer/fluorescence dye for 15 min and fluorescence was read at 480/520 nm.

### Azoxymethane-Induced Colon Carcinogenesis and ACF Analysis

Approximately six weeks old mice were divided into 4 groups (5 mice/group). Mice in groups 3 and 4 were treated with azoxymethane in sterile saline, at a dose of 10 mg/kg body weight i.p. once a week, for 3 weeks. In group 4, mice were treated with aldose reductase inhibitor sorbinil (25 mg/kg body weight per day) for entire period after 1 week from the first azoxymethane injection. Mice in groups 1 and 2 received equal volumes of sterile saline and sorbinil, respectively. Similarly aldose reductase knock out mice also were treated with azoxymethane (10 mg/kg body weight i.p.) once a week for 3 weeks. After 9 weeks all mice were killed by CO₂ euthanasia. The colons were removed, flushed with saline and opened from anus to cecum and fixed flat between two pieces of filter paper in 10% buffered formalin for 24 h. Colons were stained with 0.2% methylene blue dissolved in saline and the numbers of aberrant crypts foci (ACF) were counted under a microscope at 200× magnification.

In vivo Metastasis

For in vivo metastasis studies, 4- to 6-weeks-old male nude^{nu/nu} mice were obtained from Harlan Sprague Dawley (Indianapolis, Ind.) and housed in clean, pathogen-free rooms in an environment with controlled temperature (22° C.), humidity and a 12 hours light/dark cycle. The mice were fed standard chow (Formula Chow 5008; Purina Mills, St. Louis, Mo.), given tap water ad libitum and were allowed to acclimate for 1 week. Metastatic HT29-GFP cells were injected intrasplenically by known methods.

Briefly, mice were anesthetized with halothane, a small left abdominal flank incision was created and the spleen was exteriorized. HT29-GFP cells were harvested using only trypsin and were resuspended as a single-cell suspension in Hanks Balanced Salt Solution, free of Mg²⁺ and Ca²⁺. HT29-GFP cells (5×10⁶ cells/400 μl) were injected into the spleen with a 27-gauge needle. The spleen was returned to the abdomen and the wound was closed in one layer with wound clips. After 24 hrs spleen was removed and animals were randomized into metastatic control and aldose reductase inhibitor, sorbinil group. Control group was fed with normal diet and aldose reductase inhibitor group fed with sorbinil (40 mg/kg body weight) in the diet. Mice were killed after 35 days, and metastasis in the liver was evaluated by using the Illumatool TLS (Lightools Research, Encinitas, Calif.).

### Effect of Aldose Reductase Inhibition or Ablation on Tumor Growth in Nude Mice

Athymic nude nu/nu mice were obtained from Harlan (Indianapolis, Ind.). Nine mice (20 weeks old) were divided into three groups of three animals and were treated with PBS, scrambled aldose reductase siRNA, and AR-siRNA, respectively. All nine animals were injected with 2×10⁶ A549 human lung carcinoma cell suspensions in 100 μl PBS s.c. Animals were examined daily for signs of tumor growth. Treatment was given two times (day 1 and day 14) when the tumor surface area approximately 45 mm² (day 25). Treatment consisted of 200 μg AR-siRNA in 100 μl PBS. Control groups were treated with 200 μg/100 μl scrambled siRNA or diluent (PBS) alone. For the determination of effect of aldose reductase inhibitor, animals were fed with zopolrestat (40 mg/kg body weight) in the diet. Tumors were measured in two dimensions using calipers.

### Measurement of AR Expression in Sections of Tumor Xenografts

Serial sections (5 μM) of para-formaldehyde fixed xenografts were deparafenized and rehydrated and incubated with peptide specific aldose reductase antibodies and developed using DakoCytomation LSAB+System-HRP kit. The intensity of staining was observed under light microscope with 200× magnification.

### Western Blot Analysis

To examine expression of any of Cox-1, Cox-2, phospho-PKC-2, GAPDH, E2F1, Cyclin D1, Cyclin E, and iNOS proteins, Western blot analyses were carried by known methods (15). Equal amounts of protein from cell extracts were subjected to 12% SDS-PAGE followed by transfer of proteins to nitrocellulose filters, probing with the indicated antibodies, and the antigen-antibody complex was detected by enhanced chemiluminescence (Pierce, Piscataway, N.J., USA). Data are presented as mean±SE and P values were determined by unpaired Student's t test. P values of <0.01 were considered significant.

### EXAMPLE 3

### Effect of Aldose Reductase Inhibition on TNF-a Generation in High Glucose

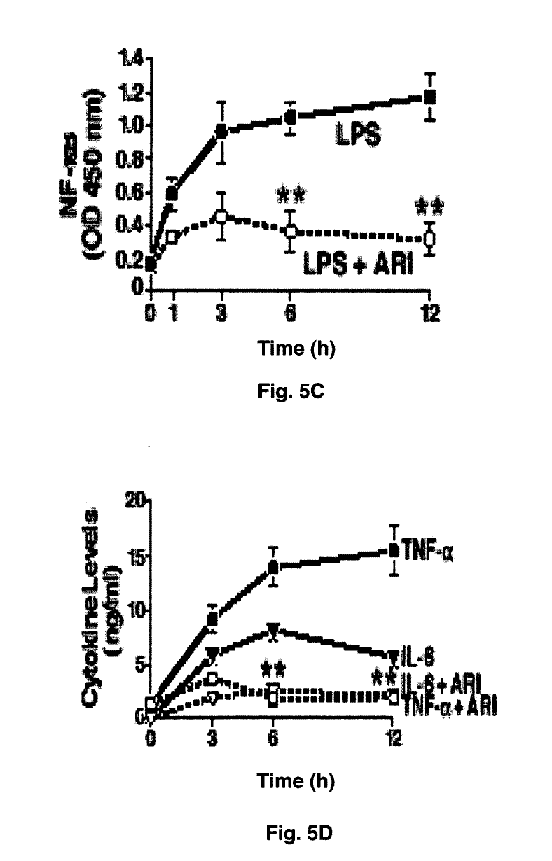

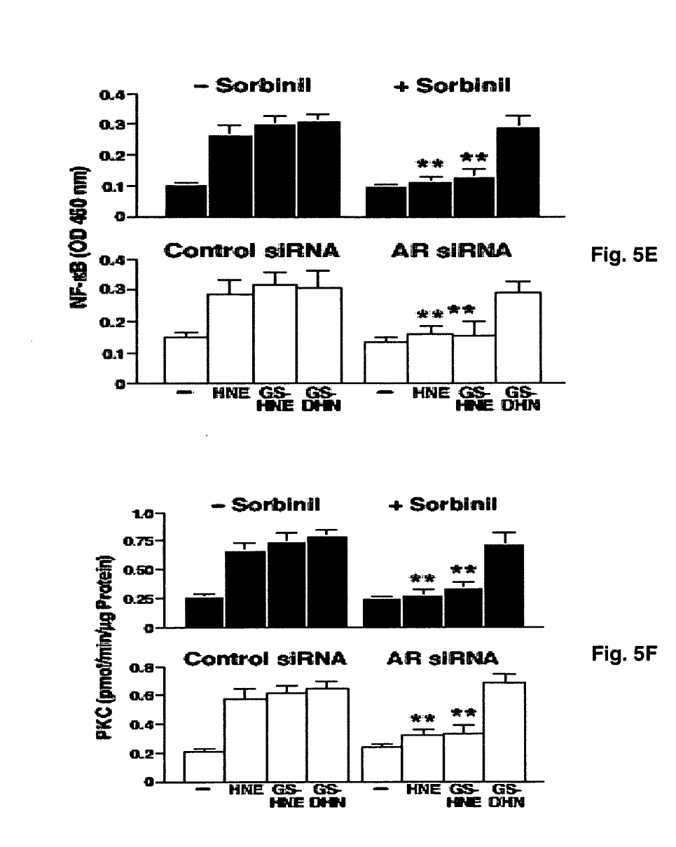

The effects of inhibiting PLC, NADPH oxidase and aldose reductase on the production of TNF-a in a culture medium (rat VSMC cells) are demonstrated. Growth-arrested VSMC in 5.5 mM glucose (NG) were preincubated for 1 h without or with apocyanin (25 mM), D609 (100 mM), calphostin C (0.2 mM), N-acetyl cysteine (10 mM) and NF-kB inhibitor (18 mM) respectively, followed by the addition of 19.5 mM glucose, after which the cells were incubated for 12 and 24 hrs. As shown in FIG. 5A, incubation with the PC-PLC inhibitor (calphostin C) markedly decreased TNF-a secretion. A similar decrease in TNF-a was observed in cells treated with the NADPH oxidase inhibitor apocyanin and the antioxidant N-acetylcysteine. Collectively, these observations support a mechanism in which high glucose increases TNF-a secretion by stimulating an intracellular signaling pathway that depends upon the activation of PLC and NADPH oxidase and the resultant change in the redox state of the cells.

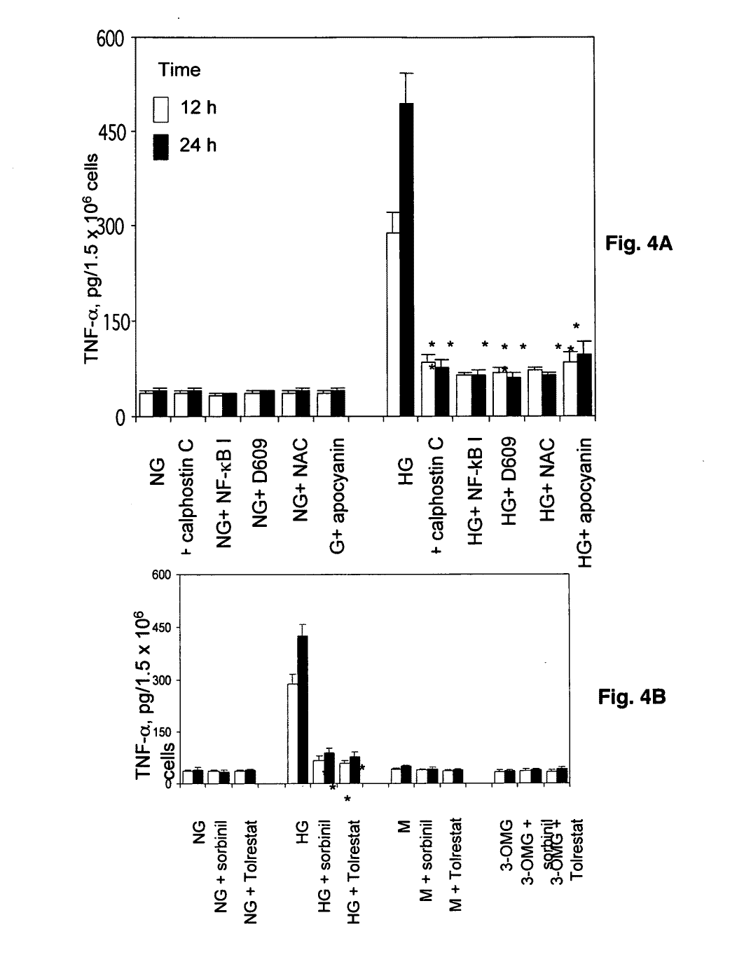

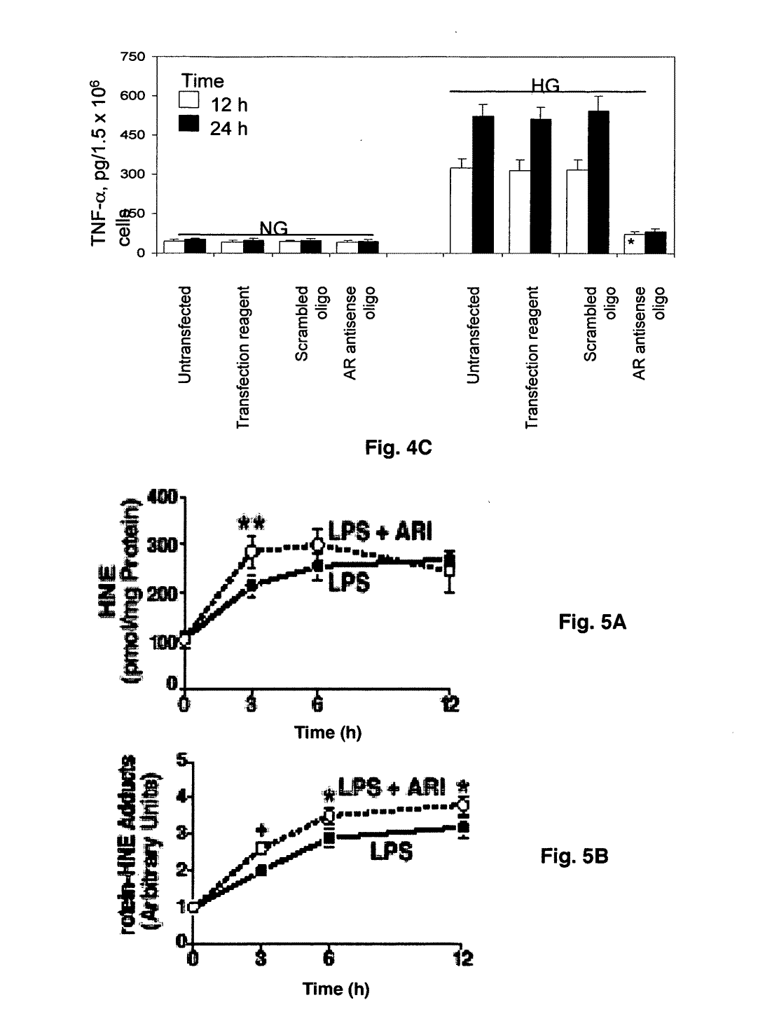

That this mechanism requires aldose reductase is suggested by data presented in FIGS. 4B-4C, which show that either pharmacological inhibition of aldose reductase by treating cells with aldose reductase inhibitors sorbinil or tolrestat or antisense ablation of the AR gene prevents high glucose-induced TNF-a secretion. Treatment with aldose reductase inhibitors did not affect basal levels of TNF-a in media containing 5.5 mM glucose, mannitol, or 3-OMG. Moreover, high glucose-induced TNF-a production was not prevented in untransfected cells or cells incubated with the transfection medium or transfection medium containing scrambled oligonucleotides. These observations attest to the specificity of TNF-a generation on aldose reductase activity. Taken together, the signaling studies described above suggest that high glucose increases TNF-a secretion, by increasing aldose reductase and phospholipase C. These processes stimulate PKC and then NF-κB, which in turn increases transcription of the TNF-a gene.

### EXAMPLE 4

### Effect of Aldose Reductase Inhibition on NF-κB Mediated Inflammatory Response Induced by Bacterial Infection

NF-κB is a central transcriptional regulator of inflammatory mediators. Reactive oxygen species (ROS) can stimulate nuclear localization and activation of NF-κB however the exact mechanism is unknown. A model of NF-κB activation induced by bacterial infection was used to study how ROS might activate NF-κB.

The effect of aldose reductase inhibition on 4-hydroxy-trans-2-nonenol (HNE) induction by bacterial lipopolysaccharide (LPS) was evaluated in RAW264.7 macrophages. LPS was found to increase HNE and protein-HNE adducts by nearly 3-fold within 6 h (FIGS. 5A-5B). When aldose reductase was pharmacologically inhibited with sorbinil, HNE and protein-HNE adduct levels increased, consistent with a role for aldose reductase in reducing HNE. Inhibition of aldose reductase significantly decreased LPS-induced NF-κB and cytokine activation (FIGS. 5C-5D). In macrophage cells, treatment with HNE/Glutathione (GS)-HNE or glutathione conjugate of 1,4-dihydroxynonene (GS-DHN) resulted in phosphorylation of IKK-a/b and translocation of NF-κB to the nucleus (FIG. 5E). Inhibition of aldose reductase with sorbinil or small interfering RNA (siRNA) targeting aldose reductase mRNA significantly blunted the effects of HNE/GS-HNE on IKK-a/b phosphorylation and NF-kB translocation, but had no effect on the ability of GS-DHN, the already reduced form of GS-HNE, to activate NF-κB (FIG. 5E), suggesting that GS-DHN is sufficient for NF-κB translocation and is involved in IKK-a/b phosphorylation.

To determine if GS-DHN serves as a cellular sensor of ROS-induced insults, its effects on phosphorylation events upstream of IKK/NF-κB activation in RAW264.7 macrophages was examined. After GS-DHN challenge, the activity of protein kinase C (PKC), a kinase upstream of IKK increased by ˜2.5 fold within 60 min (FIG. 5F). GS-DHN also induced phosphorylation of PLC-b3 and PLC-g1, which activate PKC but did not affect total PLC protein levels. HNE and GS-HNE had similar effects on the phosphorylation of the kinases upstream of NF-κB (FIG. 5F). However, pharmacologic inhibition of aldose reductase decreased the phosphorylation of PLC, PKC, and IKK induced by HNE and GS-HNE, but had no effect on GS-DHN-initiated phosphorylation of PLC and its downstream kinases (FIG. 5F). siRNA-mediated ablation of aldose reductase produced similar effects (FIG. 5F). These findings suggest that aldose reductase activity results in the production of reduced lipid aldehyde-glutathione conjugates that initiate an inflammatory cascade via PLC.

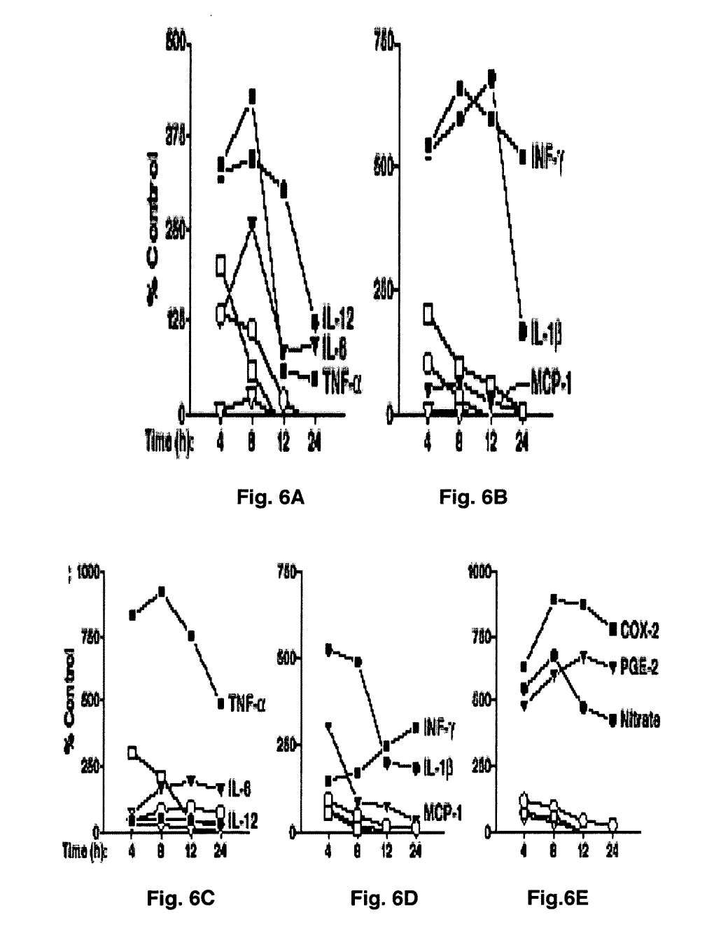

To investigate whether aldose reductase mediates the LPS signal in vivo, examined the effects of aldose reductase inhibition on NF-κB signaling pathways and myocardial dysfunction in a mouse model of overwhelming sepsis was examined. After pretreatment with sorbinil or vehicle alone, mice were injected peritoneally with a sub-lethal dose (4 mg/kg body wt) of LPS, and serum levels of inflammatory cytokines and chemokines were measured (FIGS. 6A-6B). In controls, TNF-a, IL-6, IL-12, and interferon-g levels increased 3- to 6-fold within 8 h after LPS exposure and began declining by 24 h but remained elevated. In sorbinil-treated mice, however, serum cytokine levels increased only 2-fold, began declining within 4 h, and returned to baseline levels within 12 h. Remarkably, treatment with sorbinil 2 h after LPS exposure also blunted the maladaptive systemic inflammatory response in serum. Similar effects of aldose reductase inhibitor on cytokine and chemokine activation within the myocardium after LPS challenge was also observed (FIGS. 6A, 6C).

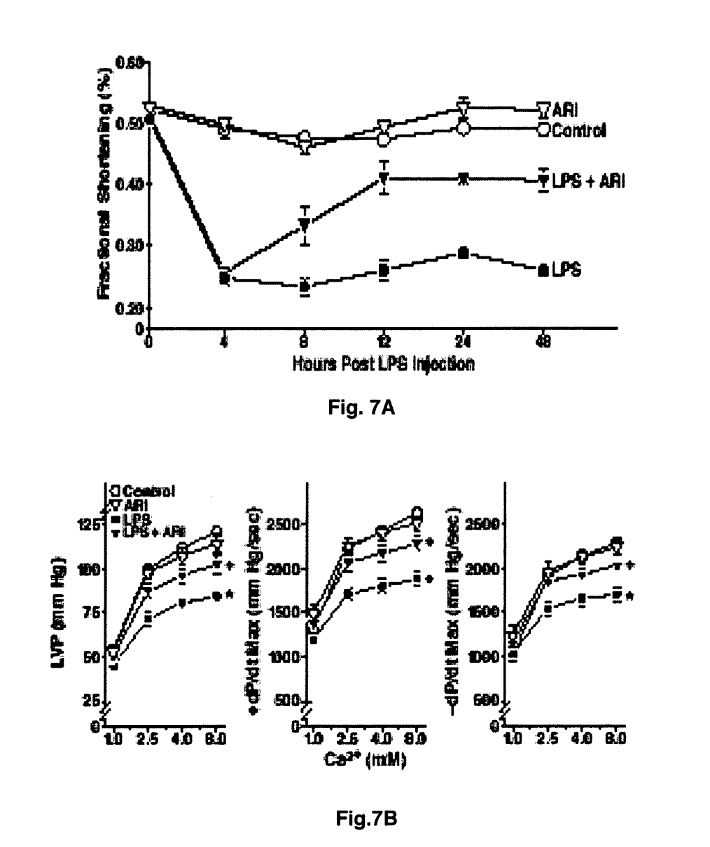

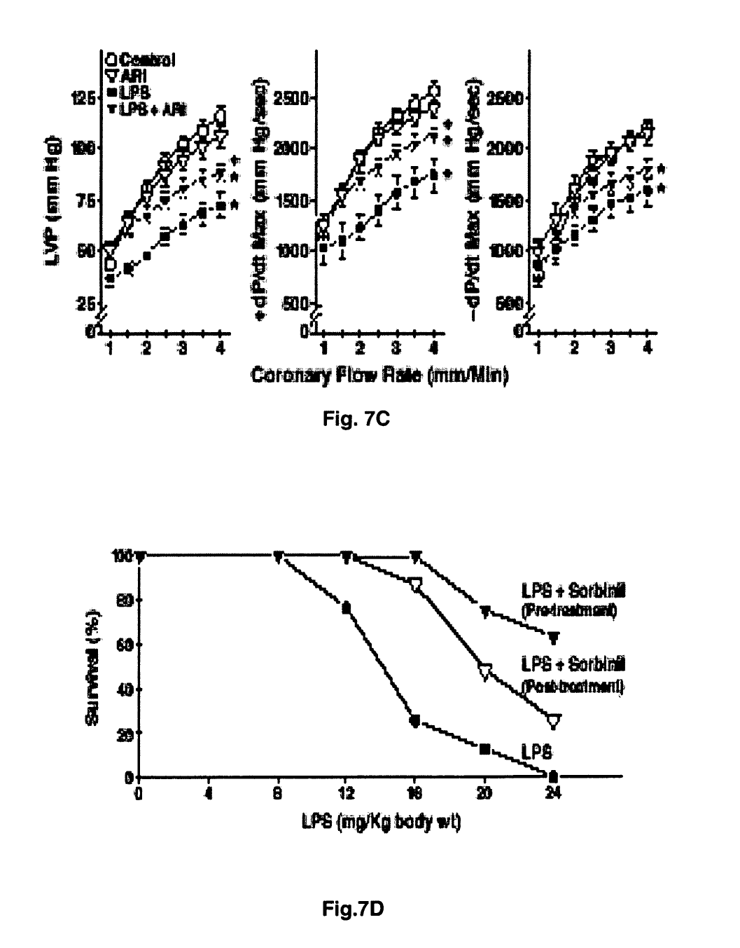

To determine if aldose reductase inhibition could also rescue the cardiac dysfunction associated with the inflammatory response, serial echocardiography in LPS-challenged mice pretreated with sorbinil or vehicle and in unchallenged controls injected with vehicle or sorbinil was performed. In all LPS-challenged mice, percent fractional shortening (FS %) was depressed at 4 h after the injection; however, at 8 h, FS % had recovered significantly in the mice pretreated with sorbinil, but had deteriorated further in vehicle-injected controls (FIG. 7A). The functional recovery in the sorbinil group persisted at 12, 24, and 48 h, but FS % in the LPS challenged animals remained profoundly depressed. The FS % was not affected in unchallenged controls. Similar results were observed in mice given sorbinil 2 h after LPS challenge. The general activity level of LPS-exposed mice was consistent with the echocardiographic findings: sorbinil-treated mice exhibited normal grooming and other activities within 24 h, while LPS-treated mice remained inactive and huddled close to one another.

To more rigorously assess the effect of aldose reductase inhibition on cardiac function, spontaneously beating isolated mouse hearts (Langendorff preparation) were perfused with the aldose reductase inhibitor and challenged with LPS (FIGS. 7B-7C). In the presence of LPS, perfusion with sorbinil significantly increased left ventricular pressure (LVP), the velocity of ventricular contraction (+dP/dtmax), and the velocity of ventricular relaxation (−dP/dtmax) compared to vehicle; the time to maximal ±dP/dt, coronary perfusion pressure, coronary vascular resistance, and heart rate were unaffected. When calcium concentration or coronary flow rate was increased, the differences in sorbinil-treated mice were further magnified (FIGS. 7B-7C). These findings demonstrate that inhibition of aldose reductase activity rapidly improved the systolic and diastolic cardiac dysfunction induced by LPS.

The above studies were performed with sublethal doses of LPS in order to assess effects on cardiac function. However, levels of LPS after bacterial sepsis often cause lethality in humans despite antibiotic therapy. Therefore increasing doses of LPS was administered to determine the dose at which fifty percent lethality occurred (LD50) in the presence or absence of aldose reductase inhibitor (ARI) in order to determine if ARI protected mice for LPS-induced death (FIG. 7D). It was found that the LD50 in control mice was 14 mg/kg LPS as previously reported. Remarkably, pre-treatment of mice with sorbinil resulted in approximately 90% survival at the same LPS dose and over 60% survival even with LPS doses as high as 24 mg/kg, which was a 100% lethal dose in controls by 48 hours. Administration of sorbinil two hours after LPS exposure, without pretreatment, still resulted in improved mortality compared to controls with an LD50 of 20 mg/kg (FIG. 7D). Thus, inhibition of aldose reductase prevented mortality associated with lethal doses of LPS.

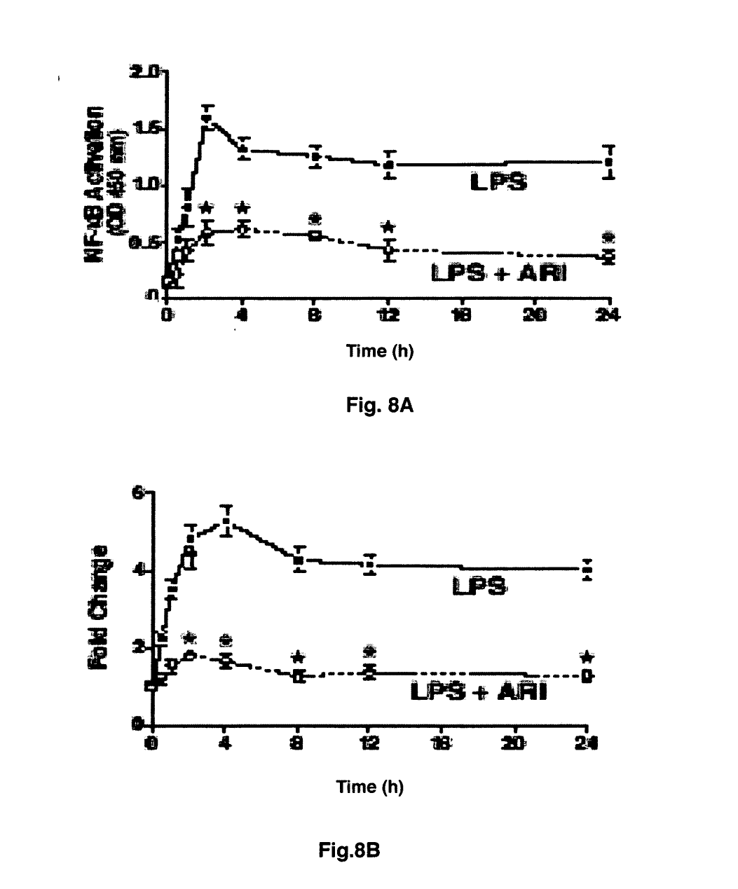

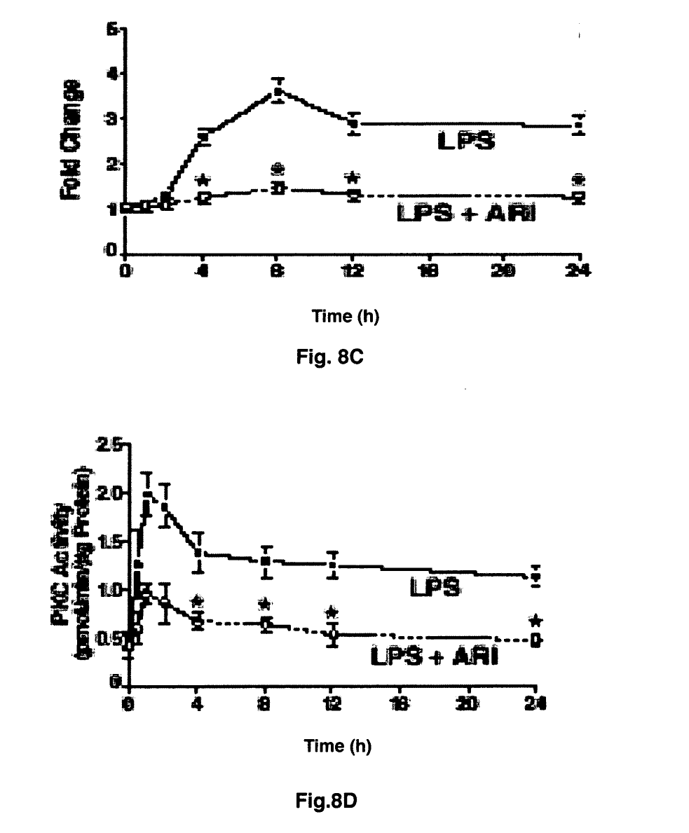

In untreated mice, LPS increased cardiac NF-κB activation by 16-fold and AP1 activation by 5-fold within 2 h, and the levels remained elevated even after 24 h (FIGS. 8A-8B). In sorbinil-treated mice, however, activation of NF-κB and AP1 decreased by ˜70% at 2 h, and the levels returned to baseline by 24 h. Similar results were observed in mice treated with sorbinil before or 2 h after LPS exposure. These findings are consistent with the downregulation of inflammatory cytokines and chemokines in the serum and heart upon aldose reductase inhibition (FIGS. 8A-8D). Further, in response to LPS, cardiac iNOS levels increased by ˜3-fold at 8 h and remained elevated at 24 h; however, in sorbinil-treated mice, iNOS levels increased only slightly and returned nearly to baseline levels by 24 h (FIG. 8C). As in macrophages, LPS significantly increased the phosphorylation of upstream kinases of NF-κB and AP1 (e.g.,MAPK, IKK, PKC, and PLC) in the heart. Aldose reductase inhibition attenuated the phosphorylation of almost all members of this cascade (FIGS. 8D-8E). Thus, aldose reductase appears to inhibit the inflammatory cascade by regulating the activation of NF-κB, thereby protecting against cardiovascular collapse in the setting of overwhelming sepsis.

### EXAMPLE 5

In vitro Effects of Aldose Reductase Inhibition on Caco-2, HT-29 and A549 Cell Lines

### Aldose Reductase Inhibition Prevents PGE2 Production and Cox Activity Caco-2 Cells

The growth factors are known to induce PGE2 production by activating inducible Cox-2 in colon cancer (58), but the mechanism is not well understood. Inhibition of aldose reductase significantly (>90%) prevented the production of PGE2 by Caco-2 cells induced by BFGF and

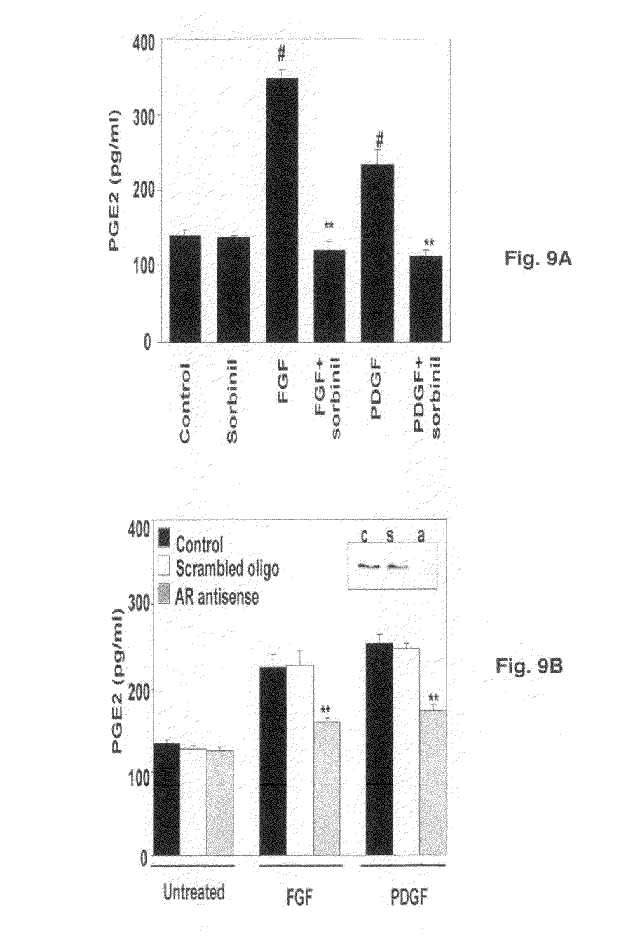

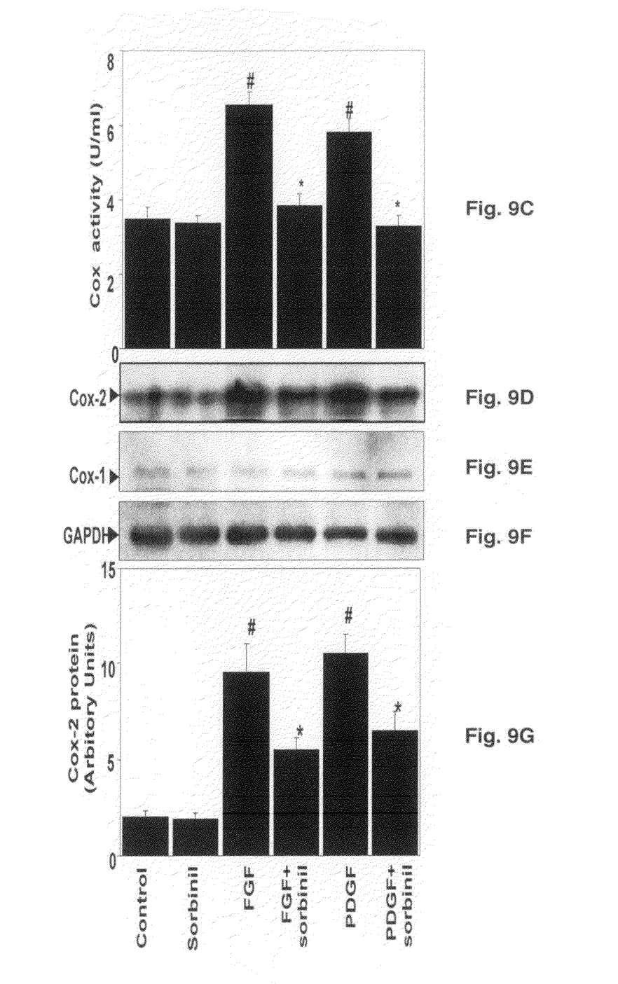

PDGF (FIG. 9A). However, sorbinil alone did not inhibit constitutive levels of PGE2. Since the non-specificity of aldose reductase inhibitors could not be rigorously excluded, parallel studies were performed by transfecting Caco-2 cells with antisense aldose reductase oligonucleotides that decreased aldose reductase protein expression by >95% (FIG. 9B, inset) and also the enzyme activity by >90% (data not shown). In contrast to the cells transfected with scrambled oligonucleotides, cells transfected with antisense aldose reductase displayed markedly attenuated PGE2 production upon stimulation with BFGF or PDGF (FIG. 9B). PGE2 generation in Cox-2 negative cells (HCT-116) by growth factors was non-significant (data not shown).

Since PGE2 is synthesized from its precursor arachidonic acid catalyzed by cyclooxygenases, whether or not inhibition of aldose reductase prevents growth factor-induced expression of Cox enzymes was examined. Treatment of Caco-2 cells with BFGF and PDGF significantly (60-80%) increased Cox activity (FIG. 9C). Pre-incubation with sorbinil abolished both BFGF and PDGF-induced Cox activity. Since Cox activity is contributed by two isozymes, constitutive Cox-1 and inducible Cox-2, the affect of aldose reductase inhibition on Cox-1 and Cox-2 isozymes was examined by Western blot analysis using specific antibodies. The levels of constitutive Cox-1 protein were not affected by growth factors or sorbinil (FIG. 9E), whereas Cox-2 protein significantly increased and was attenuated by sorbinil (FIGS. 9D, 9G).

### Aldose Reductase Inhibition Prevents HT-29 and A549 Cell Proliferation

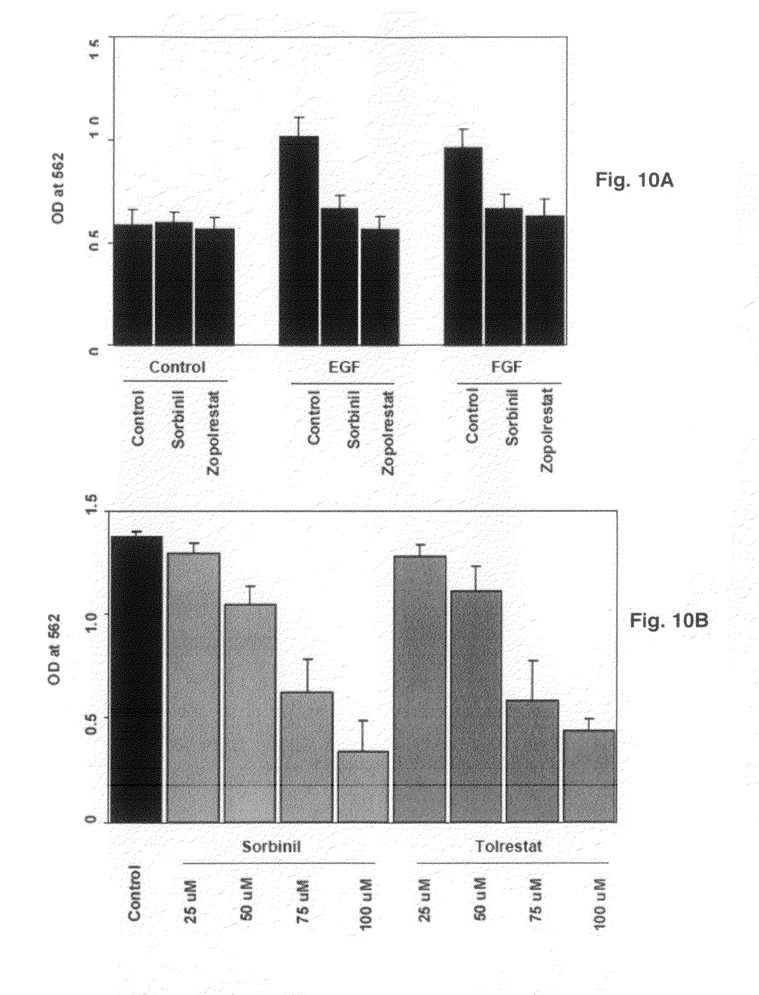

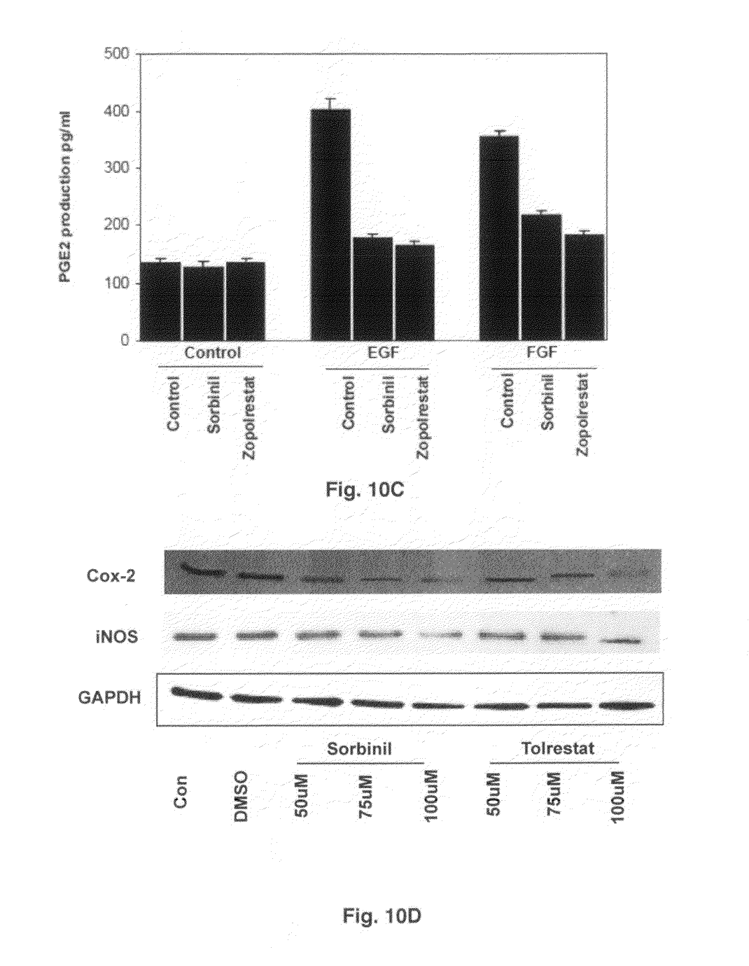

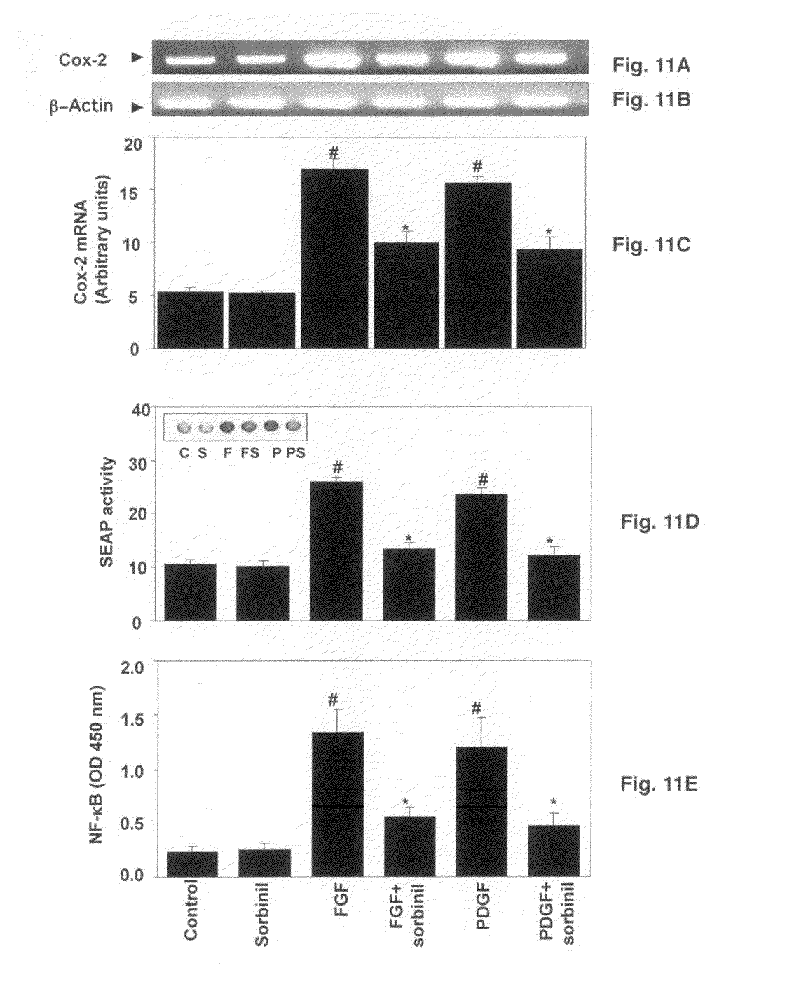

The effect of aldose reductase inhibitors sorbinil and zopolresta on the role of aldose reductase in the signal transduction pathway of growth factors leading to HT29 cells proliferation is examined. The extent of HT29 cells proliferation was determined by MTT assay. FIG. 10A demonstrates that treatment of HT29 cells with EGF and BFGF for 24 h significantly (>40%) stimulated growth. The increase in HT29 cell growth was significantly attenuated (>60%) by aldose reductase inhibitors, sorbinil or zopolrestat. However, sorbinil or zopolrestat alone did not cause any effect on HT29 cells proliferation. It is contemplated that aldose reductase is an obligatory mediator of growth factors-induced colon cancer cell proliferation.

The extent of A549 cells proliferation was determined by MTT assay. FIG. 10B demonstrates that treatment of A549 cells with various concentrations of sorbinil and tolrestat resulted into concentration dependent inhibition of proliferation. The maximum 100 μM concentration of sorbinil or tolrestat caused more than 75% inhibition of A549 cells proliferation. It is contemplated that aldose reductase is an obligatory mediator in the lung cancer cells proliferation.

The role of aldose reductase in the induction PGE2 production by growth factors in HT29 cells also is examined. FIG. 10C shows that both EGF and BFGF significantly induced the production of PGE2 by HT29 cells and aldose reductase inhibitors significantly (>90%) prevented it. However, sorbinil or zopolrestat alone did not inhibit constitutive levels of PGE2. It is contemplated that aldose reductase inhibition prevents growth factors-induced PGE2 production in HT29 cells.

To determine the role of aldose reductase in inflammatory markers expression expression of Cox-2 and iNOS in A549 cells is measured. FIG. 10D shows that inhibition of aldose reductase significantly prevents the expression of Cox-2 and iNOS proteins in a concentration dependent manner. It is contemplated that aldose reductase inhibitors could be used as therapeutic drugs in inflammation related cancers such as lung cancer.

### Inhibition of AR Prevents Growth Factor-Induced NF-kB Activation in Caco-2 Cells

The effect of aldose reductase inhibitors on growth factor-induced NF-κB activation was examined, because it is known that redox sensitive transcription factor NE-κB transcribes Cox-2 DNA (59) and it has been demonstrated that aldose reductase inhibition prevents growth factors and cytokine-induced NF-κB activation (15). Treatment of caco-2 cells with BFGF or PDGF significantly (2-3 fold) increased the mRNA levels of Cox-2 and sorbinil prevented it by 55-65% (FIGS. 11A-11C) suggesting that aldose reductase could regulate the transcriptional activation of Cox-2 DNA. Both BFGF and PDGF significantly (˜3 fold) induced NF-κB-dependent reporter (SEAP) activation in Caco-2 cells and sorbinil caused >60% inhibition (FIG. 11D). However, sorbinil alone did not affect the NF-κB-SEAP activity. Stimulation of Caco-2 cells with BFGF or PDGF resulted in a pronounced (˜10 fold) activation of NF-kB DNA binding activity as determined by colorimetric, non-radioactive NF-κB p65 transcription assay method (FIG. 11E) and sorbinil caused >70% inhibition.

These results validate previous measurements of NF-κB activity and substantiate that the specific activity observed in SEAP and colorimetric methods is due to NF-κB activation. It is contemplated that inhibition of aldose reductase prevents growth factor-induced activation of NF-κB in Caco-2 cells, which transcriptionally may activate Cox-2 expression.

### Inhibition of Aldose Reductase Prevents Growth Factors-Induced PKC Activation in Caco-2 Cells

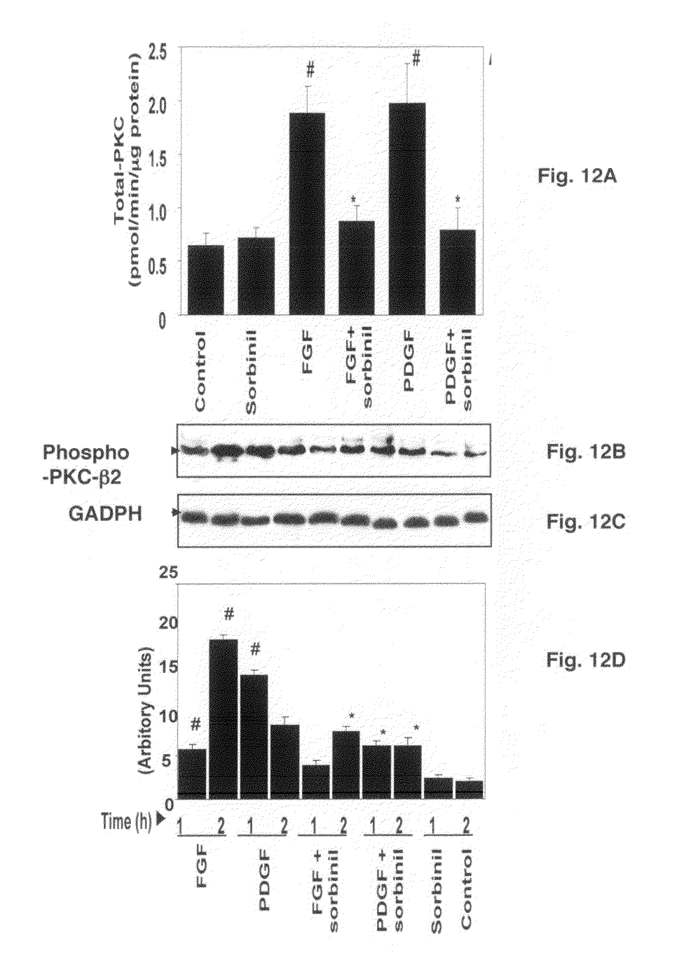

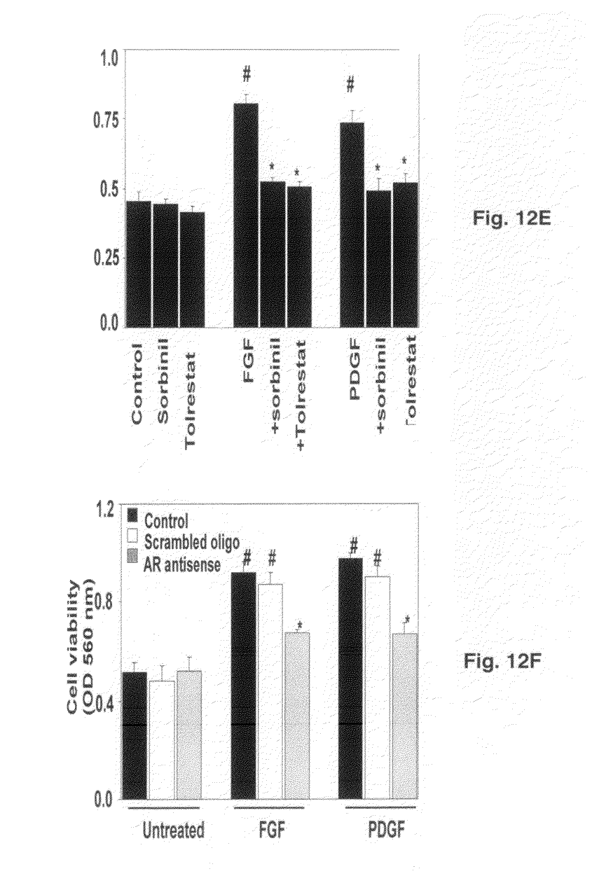

Since PKC is an upstream kinase for the activation of NF-κB and activation of PKC-b2 has been implicated in colon carcinogenesis (60), the effect of growth factors on total PKC activity in Caco-2 cells in the absence and presence of aldose reductase inhibitor was examined. Stimulation with growth factors led to a significant (˜3 fold) increase in membrane-bound PKC activity (FIG. 12A) and sorbinil significantly prevented it. However, sorbinil by itself did not alter the total PKC activity in these cells. Both BFGF and PDGF activated PKC-β2 in Caco-2 cells (FIGS. 12B-12D). BFGF caused maximal PKC phosphorylation at 2 h whereas PDGF caused maximal phosphorylation at 1 h and increase in PKC-β2 phosphorylation was significantly (>70%) attenuated by sorbinil.

### Attenuation of Growth Factors-Induced Caco-2 Cell Line Proliferation

Since increased Cox-2 expression has been shown to facilitate colon cancer progression by stimulating cell proliferation and survival (61), the role of aldose reductase in growth factors-induced Caco-2 cell growth was examined. Treatment of Caco-2 cells with BFGF and PDGF for 24 h significantly (>40%) stimulated growth (FIG. 12E) which was significantly attenuated (>80%) by sorbinil or by antisense ablation of aldose reductase (FIG. 12F) indicating that aldose reductase is an obligatory mediator of growth factors-induced colon cancer cell proliferation.

Aldose Reductase Inhibition Affects Cell Cycle and/or Cell Proliferation in Caco-2 and A549 Cells

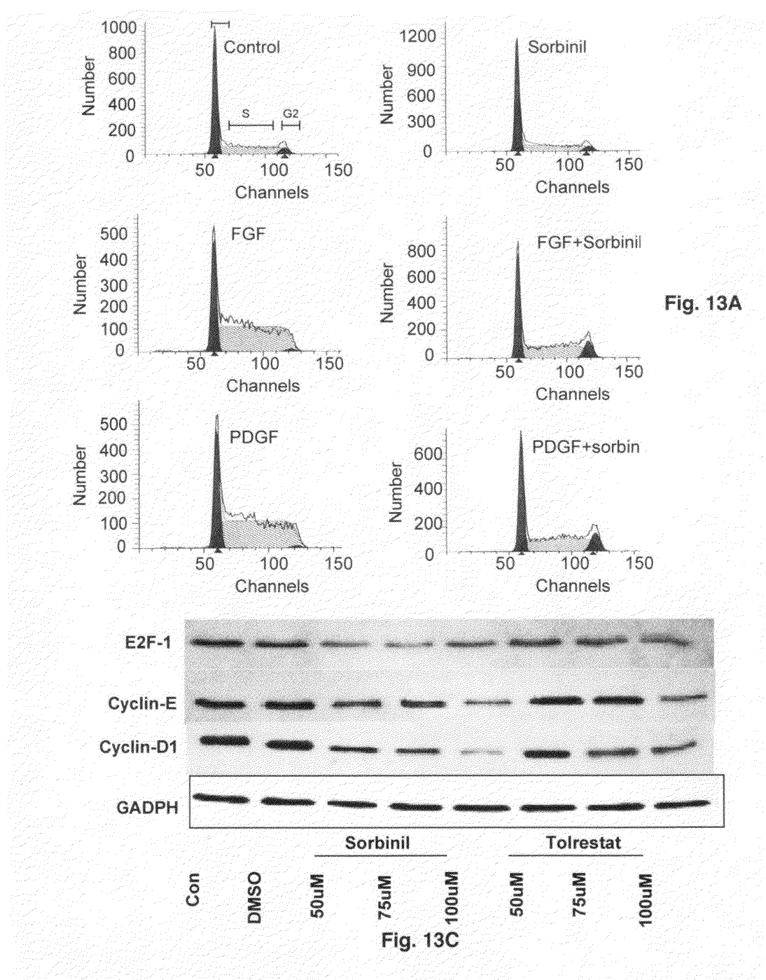

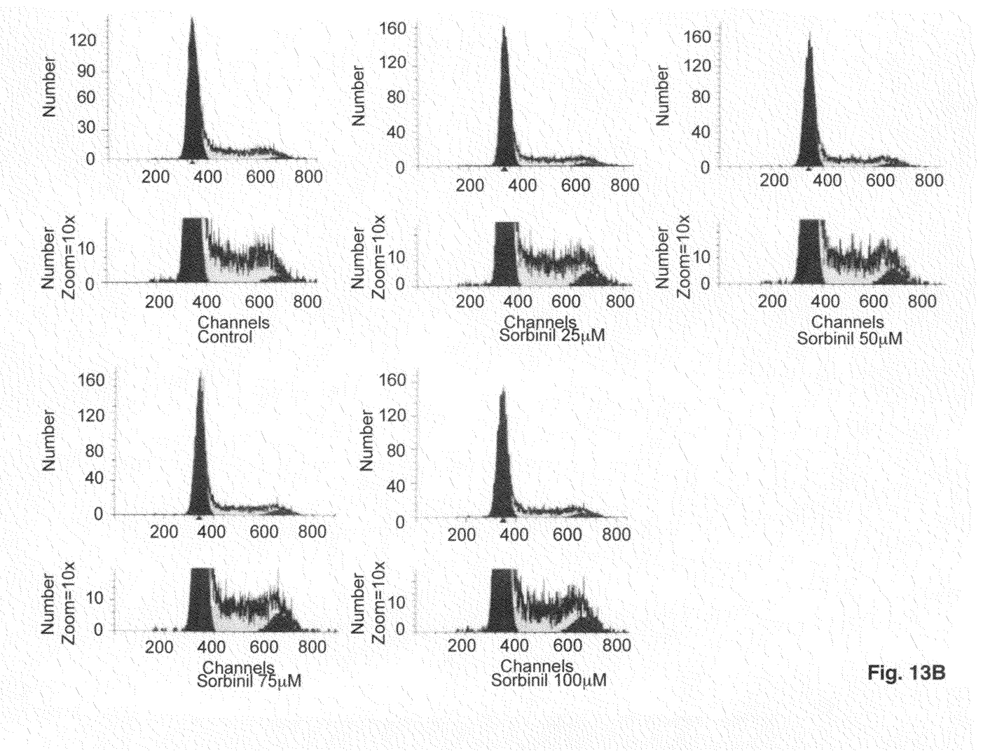

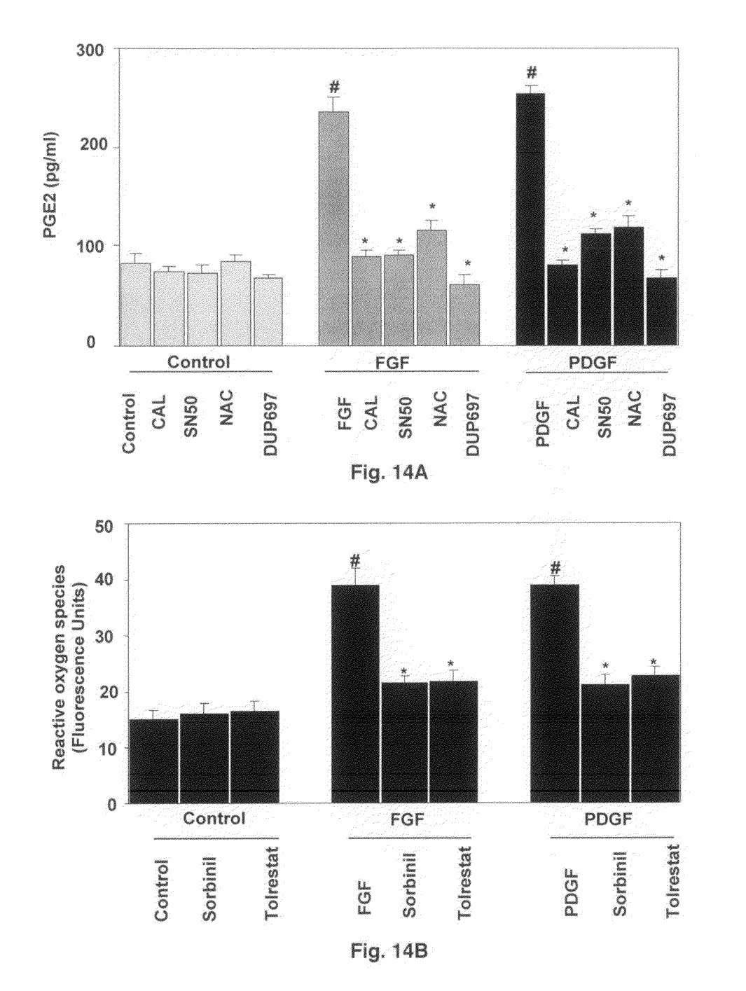

Treatment of Caco-2 cells with growth factors significantly induced synthetic (S)-phase of the cell cycle (FIG. 13A) suggesting that the cells were undergoing proliferation. Inhibition of aldose reductase prevented growth factor-induced accumulation of cells in S-phase and the cells accumulated at G2/M phase and G1 phase, suggesting that aldose reductase inhibition prevents synthetic phase of cell cycle which is an important stage required for cell growth. Table 3 shows the percentage of Caco-2 cells in the G1, G2 and S phases in the presence of sorbinil.

TABLE 3
Cell Cycle Analysis

| | | |
|---|---|---|
| G1 | S | G2 |

| | EXP | SOR | EXP | SOR | EXP | SOR |
|---|---|---|---|---|---|---|
| Control | 52.61 | 54.97 | 41.66 | 39.71 | 5.73 | 5.32 |
| FGF | 26.87 | 36.31 | 71.66 | 52.15 | 1.47 | 1.54 |
| PDGF | 27.36 | 36.18 | 71.49 | 51.71 | 1.15 | 12.10 |

Treatment of A549 cells with 10% FBS in the medium significantly induced synthetic (S) phase of cell cycle (FIG. 13B), suggesting that the cells were undergoing proliferation. Inhibition of aldose reductase prevented accumulation of cells in S phase and the cells accumulated at G2-M phase and G1 phase, suggesting that aldose reductase inhibition prevents synthetic phase of cell cycle, which is an important stage required for cell growth. Table 4 shows the percentage of A549 cells in the G1, G2 and G2 phases in the presence of sorbinil.

TABLE 4
Cell Cycle Analysis

| Medium | G1 | G2 | S |
|---|---|---|---|
| 10% FBS | 56.54 | 4.53 | 38.93 |
| Sorbinil (25 μM) | 58.79 | 6.06 | 35.14 |
| Sorbinil (50 μM) | 59.24 | 5.47 | 35.29 |
| Sorbinil (75 μM) | 64.21 | 5.55 | 30.24 |
| Sorbinil (100 μM) | 65.55 | 4.8 | 29.75 |

Treatment of A549 cells with various concentrations of sorbinil or tolrestat caused significant inhibition of important cell cycle regulatory proteins such as E2F-1, Cyclin E and Cyclin D1expression (FIG. 13C). These results indicate that inhibition of aldose reductase prevents G1 to S phase transition.

### Attenuation of Growth Factors-Induced Upregulation of PGE2 Production by Inhibitors of Signaling Cascade for NF-κB Activation

In order to understand the role of NF-κB in the growth factor-induced upregulation of PGE2, inhibitors of PKC (Calphostin c), Cox-2 (DUP697), reactive oxygen species scavenger (N-acetyl cysteine), and NF-κB (SN50) were utilized. Growth factors caused a pronounced increase in the production of PGE2 and preincubation of Caco-2 cell with the above inhibitors attenuated, indicating that signaling events that lead to activation of NF-κB and its dependent Cox-2 expression are involved in the production of PGE2 (FIG. 14A). Further, growth factors caused pronounced increase in ROS which was inhibited by sorbinil and tolrestat (FIG. 14B).

### Effect of Aldose Reductase Inhibition on Lipid Aldehyde-Induced Signaling in Caco-2 Cells

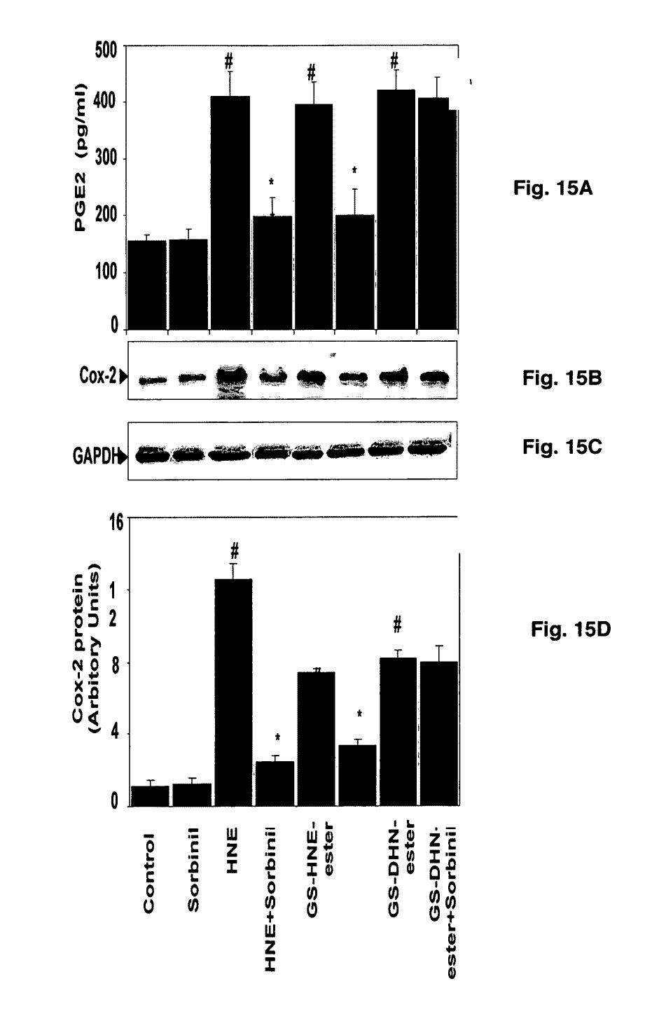

It has been demonstrated that aldose reductase is an excellent catalyst for the reduction of lipid peroxidation-derived aldehydes, such as HNE and their conjugates with glutathione to corresponding alcohols (4, 20). Since, it is contemplated that aldose reductase inhibition or ablation prevents growth factor-induced expression of Cox-2 and production of PGE2, AR-catalyzed reduction of lipid aldehydes involvement in this mechanism was determined. Treatment of cells with HNE or cell permeable esters of GS-HNE or GS-DHN resulted in increased PGE2 production (FIG. 15A) and also Cox-2 expression (FIGS. 15B-15D). Inhibition of aldose reductase by sorbinil significantly prevented the HNE and GS-HNE-induced Cox-2 expression and PGE2 production but had no effect on GS-DHN-induced expression of these inflammatory markers. These results indicate that growth factors-induced mitogenic signaling in colon cancer cells could be mediated by the reduced form of lipid aldehyde-glutathione conjugates catalyzed by aldose reductase.

### Inhibition of Aldose Reductase Prevents Invasion of HT29 and A549 Cells

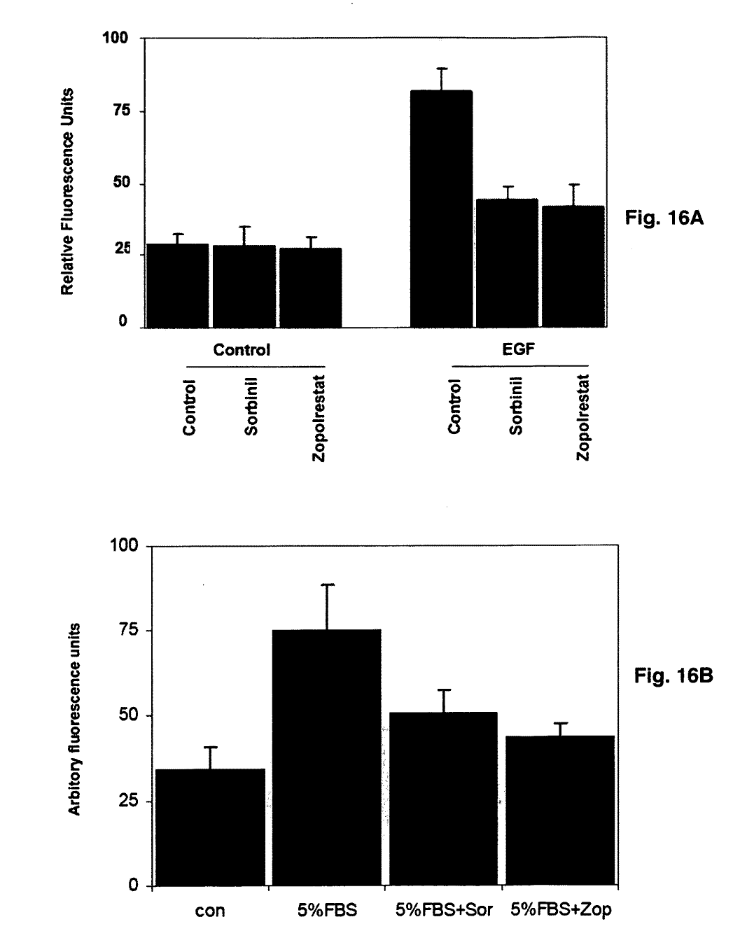

The ability of HT29 cells and A549 cells to penetrate extracellular matrix material (Matrigel) in vitro was assessed by inhibiting aldose reductase in presence of sorbinil or zopolrestat (75 μM, HT29 or 100 μM, A549). FIG. 16A shows that stimulation of HT29 cells with EGF (5 ng/ml) for 24 hrs caused significant invasion through matrigel. Inhibition of aldose reductase prevented invasion of HT29 cells by more than 70%. FIG. 16B shows that treatment of A549 cells with 5% FBS for 24 hrs caused significant invasion through matrigel. Inhibition of aldose reductase prevented invasion of A549 cells by more than 60%.

### EXAMPLE 6

In vivo Effects of Aldose Reductase and its Inhibition in Mouse Models of SW480, HT29 and A549 Cancers
Effect of Aldose Reductase siRNA on SW480 Xenografts

Athymic nude nu/nu mice were obtained from Harlan, Indianapolis, Ind. Nine 20-weeks-old athymic nu/nu nude mice were divided into three groups of 3 animals (Group 1: treated with PBS; Group 2: treated with scrambled siRNA and Group 3: treated with aldose-reductase siRNA). An aliquot of 2×10⁶ SW480 human colon adenocarcinoma cell suspensions in 100 μl PBS was injected subcutaneously into one flank of each nu/nu nude mouse. Animals were examined daily for signs of tumor growth. Treatment was administered when the tumor surface area exceeded 45 mm², i.e., day 25. Treatment consisted of 200 mg aldose-reductase siRNA in 100 ml PBS administered intraperitoneally. Control groups were treated with 200 mg/100 ml scrambled siRNA, or diluent (PBS) alone. Mice were treated on days 1 and 14. Tumors were measured in two dimensions using calipers over 40 days.

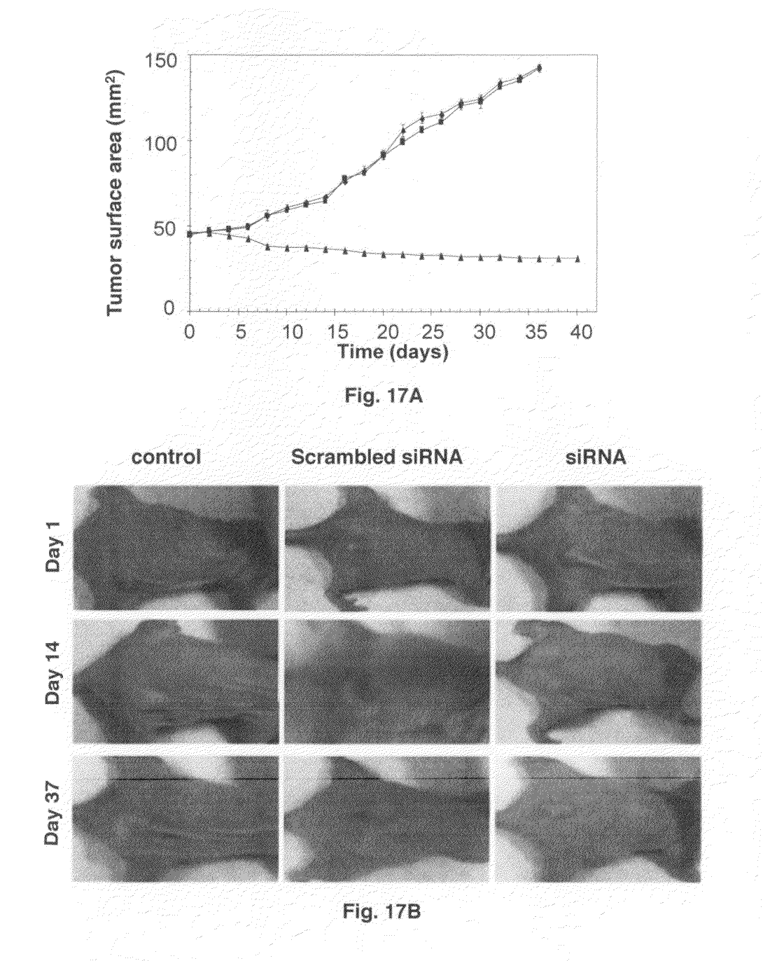

Results presented in FIG. 17A clearly demonstrate that the tumor progression was completely arrested in the animals treated with ARsiRNA, whereas uncontrolled growth was observed in the control as well as in scrambled siRNA treated mice. None of the treatments interfered with the normal weight gain of animals during the experiments. FIG. 17B are photographs of animals taken at 1, 14 and 37 days. These findings indicate that aldose reductase inhibition completely halts the colon cancer progression without interfering with the normal weight gain of the animals after its administration.

### Role of Aldose Reductase in the Prevention of Colon Cancer in AOM-Treated Wild Type and AR Knock Out Mice

Azoxymethane-induced colon carcinogenesis was studied in a mouse model. Similar to humans, azoxymethane-induced aberrant crypt foci (ACF) formation in rodent models is the earliest identifiable preneoplastic lesions in the progression of normal colonic epithelium. In addition, azoxymethane reproducibly induces aberrant crypt foci and colon tumors formation in rodents with many of the same genetic and signal transduction defects identified in human colon carcinomas.

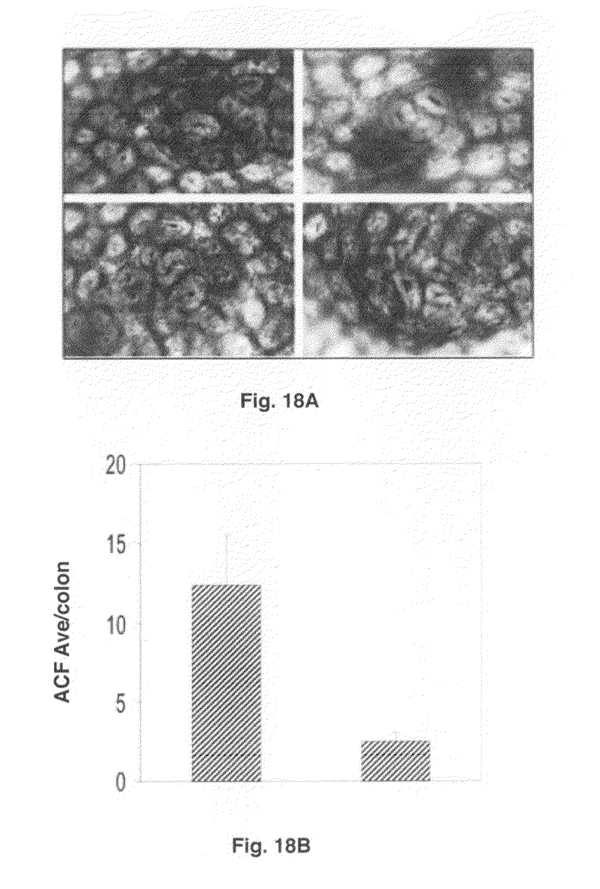

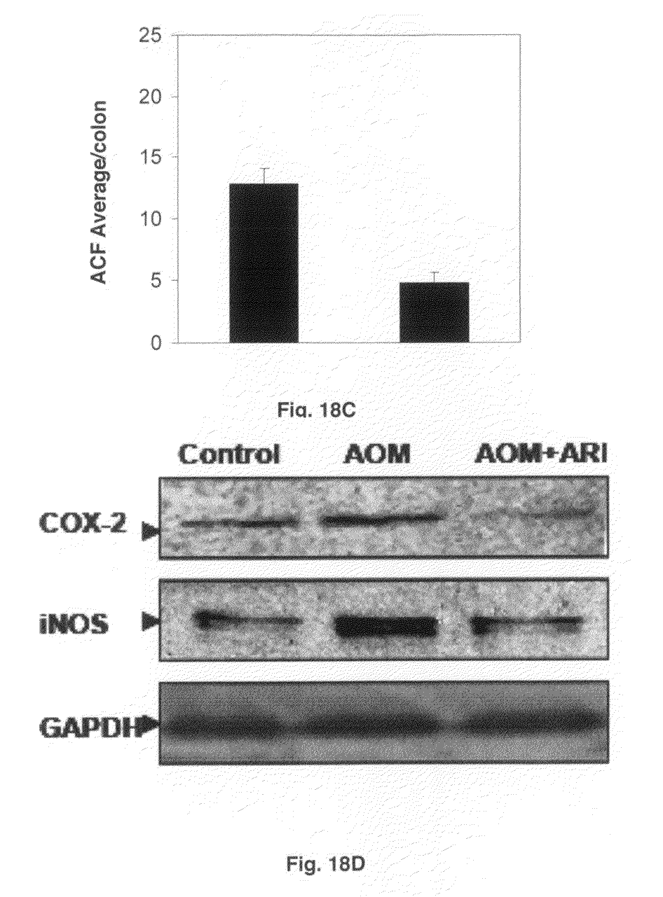

BALB/C mice were injected with azoxymethane or saline and treated with and with out aldose reductase inhibitor, sorbinil as described in Example 2. At the early preneoplastic stage (9 weeks after first azoxymethane injection), mice were sacrificed and their colons were removed and analyzed microscopically for the presence of ACF. ACF were distinguished from the surrounding normal crypts by increased thickening of the crypt walls and aberrant change in the shape of the crypt lumen (FIG. 18A). The parameters used to assess the aberrant crypt foci were their occurrence/colon and number of aberrant crypts foci. All the colons were scored by three blind observers azoxymethane did not know the identity of the samples.

FIG. 18B shows that in azoxymethane group the number of average ACF/colon were 12.4±3.2, whereas in azoxymethane+sorbinil treated mice the formation of aberrant crypt foci was significantly less (2.4±0.5), suggesting that inhibition of aldose reductase prevents azoxymethane-induced aberrant crypt foci formation. No aberrant crypt foci were observed in saline treated control animals. To rule out the non specificity of pharmacological aldose reductase inhibitor, sorbinil genetically aldose reductase gene knocked out (KO) mice were used for azoxymethane-induced aberrant crypt foci formation. FIG. 18C shows that aldose reductase KO mice had low number of aberrant crypt foci (5.2±0.8) as compared wild type mice (12±1.4). These results support the inhibition of aldose reductase preventing azoxymethane-induced aberrant crypt foci formation in wild type mice as well as aldose reductase KO mice.

To determine the role of aldose reductase in AOM-induced inflammatory markers expression the expression of Cox-2 and iNOS in mice colons after 9 weeks of AOM induction were measured. FIG. 18D shows that inhibition of aldose reductase significantly prevents AOM-induced inflammatory markers such as Cox-2 and iNOS. Aldose reductase inhibitors should be excellent chemopreventive drugs to treat colon cancer.

### Inhibition of Aldose Reductase Prevents Metastatic Tumor Growth in a Mouse Model

Liver is the common site for systemic metastasis during advanced stage of colorectal cancer. The effect of aldose reductase inhibition in tumor cell migration was examined using a mouse liver metastasis model. HT29 cells (5×10⁶), which are transfected with a plasmid containing GFP, were injected intrasplenically into the athymic mice. Animals were randomized into 2 experimental groups (5 animals per group) to receive control and sorbinil (40 mg/kg/body weight) diet. Metastasis to the liver was followed up periodically using Illumatool TLS. Detectable levels of liver metastasis were observed 4 weeks after splenic injection of HT29 cells.

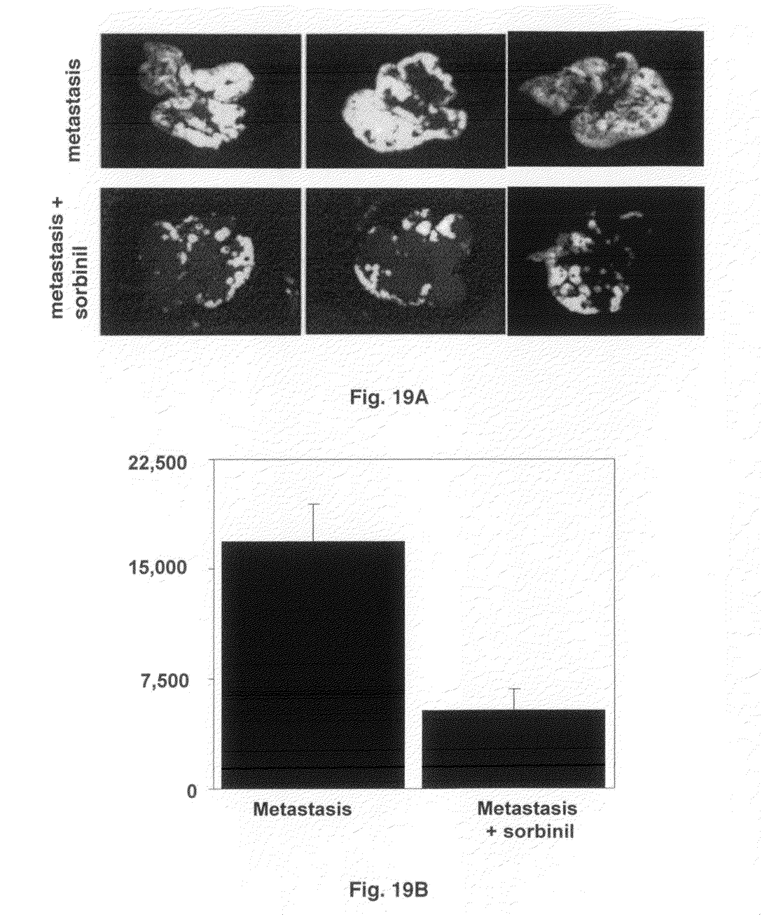

After 34 days mice were killed and development of liver metastasis was monitored by a qualitative assessment of GFP fluorescence using bioluminescent imaging. Mice fed with control diet increased metastases significantly compared to diet containing aldose reductase inhibitor, sorbinil (FIG. 19A). The results were further quantified by measurement of fluorescence and values expressed as pixel numbers (FIG. 19B). Results demonstrate a significant decrease in tumor metastasis in the sorbinil fed animals compared with control diet which correlates with the qualitative assessment. It is contemplated that inhibition of aldose reductase represents a unique strategy for the suppression of colorectal cancer metastasis.

### Aldose Reductase Inhibition Prevents Progression of Lung Cancer Tumor Growth

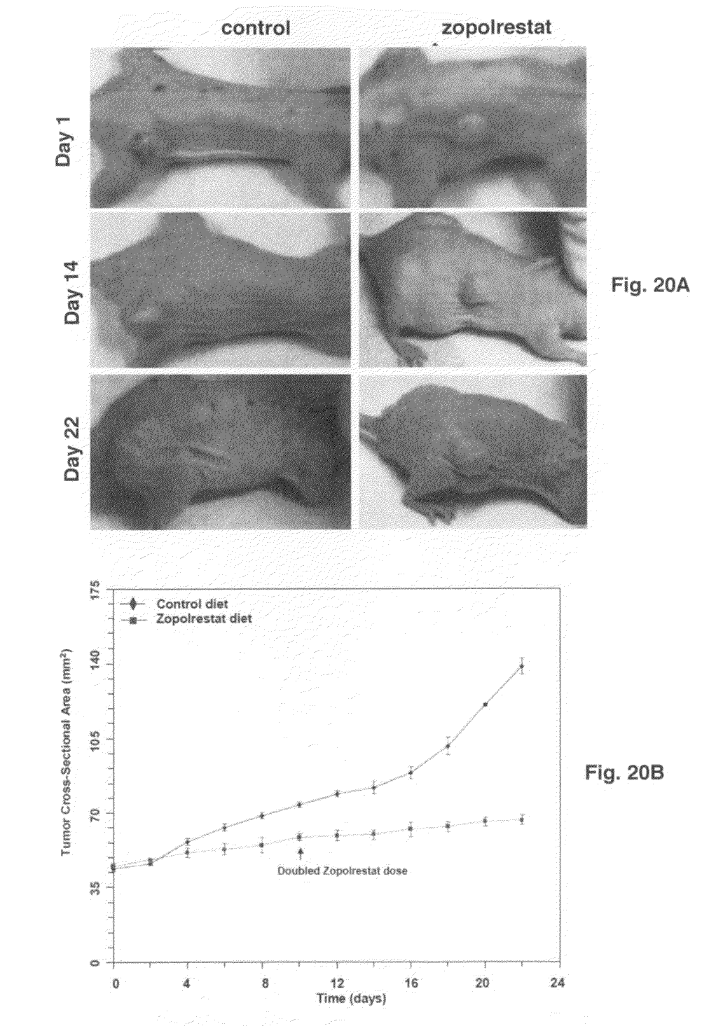

The results obtained from in vitro studies were confirmed by in vivo nude mice model bearing human lung carcinoma A549 cells. A549 (2×10⁶) cells were implanted s.c. and allowed to grow in nu/nu nude mice to ˜45 mm² over a period of 25 days. Animals were grouped into control and experimental groups. Control group were fed with regular diet and experimental group was fed with aldose reductase inhibitor, Zopolrestat (after 10 days dose increased from 20 mg to 40 mg/kg/body weight) for until end of the experiment. Tumor growth was measured every two days using calipers. The photographs of animals were taken at days 1, 14, and 37 (FIGS. 20A-20B).

FIG. 20B clearly shows that the tumor progression was completely arrested in the animals fed with aldose reductase inhibitor, zopolrestat, whereas uncontrolled growth was observed in the control animals. None of the treatments interfered with the normal weight gain of animals during the experiments.

Inhibition of Aldose Reductase by siRNA Prevents Tumor Growth and ROS Production

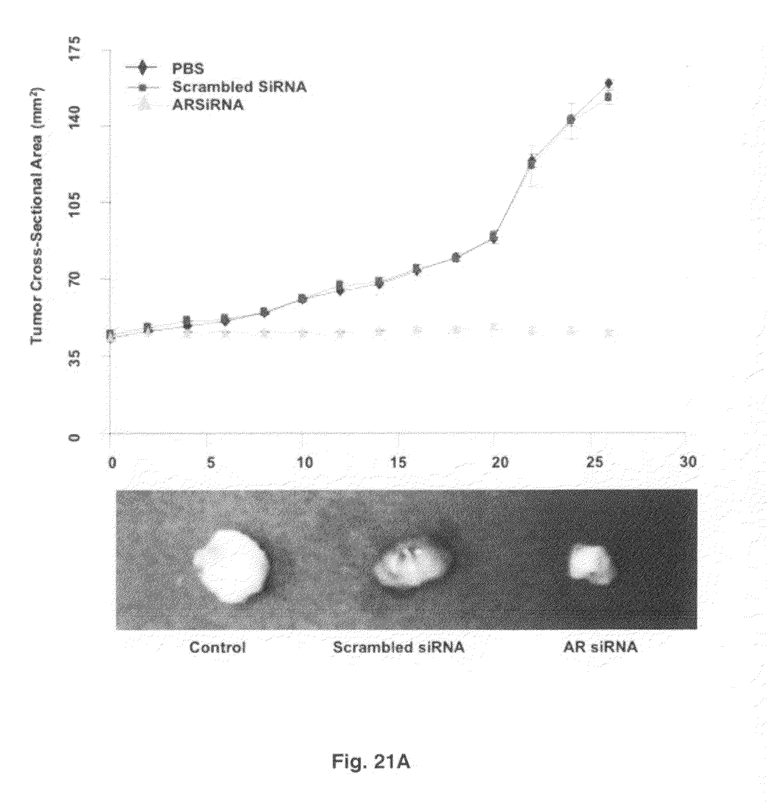

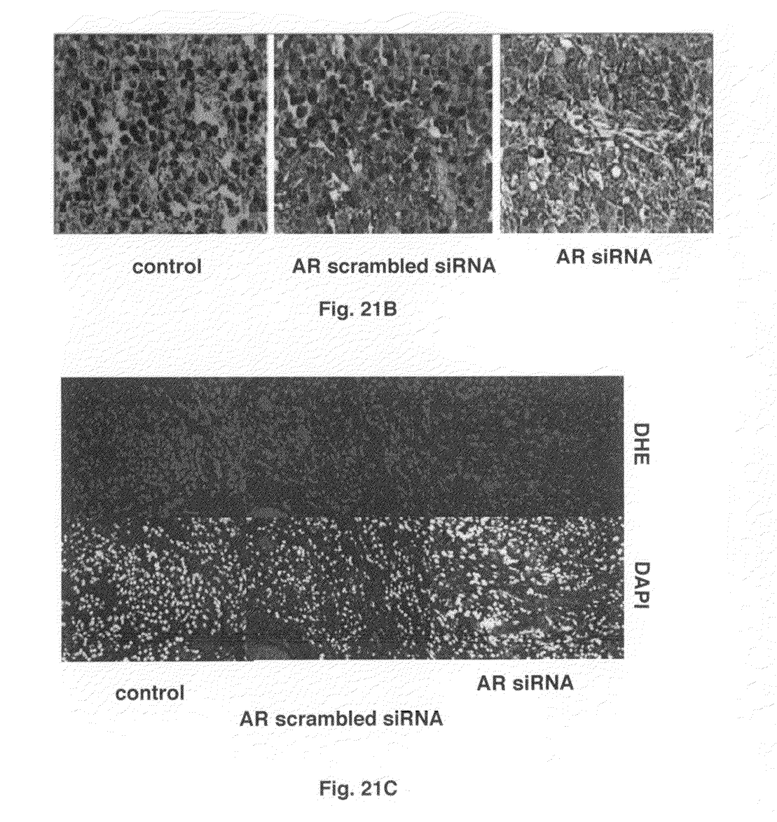

Although the pharmacological aldose reductase inhibitor zopolrestat selectively inhibits aldose reductase, the nonspecificity of this drug could not be rigorously excluded. Therefore, the role aldose reductase in lung cancer tumor progression was confirmed by ablating aldose reductase with siRNA. FIG. 21A shows that mice which received i.p, injection of 200 μg AR-siRNA in 100 μl PBS on days 1 and 14 the progression of tumor growth was inhibited completely compared to animals which received an injection PBS, scrambled siRNA.

To confirm that aldose reductase inhibition prevents aldose reductase protein expression in the xenografts, serial sections of the xenograft were taken and fixed in para-formaldehyde as in Example 4. Cross sections of control, scrambled and siRNA injected nude mice tumors were stained with antibodies against peptide specific aldose reductase. FIG. 21B demonstrate animals treated with AR-siRNA showed significantly prevention of aldose reductase protein expression compared to control and scrambled siRNA as evidenced by dark brown color. These results indicate that inhibition of aldose reductase prevents tumors progression in lung cancer xenografts.

Since progression of tumorigenesis is usually induced by reactive oxygen species (ROS) generation, whether inhibition of aldose reductase prevents ROS production was measured in nude mice xenograft sections. The tumor sections were prepared and red fluorescence in the presence of HEt dye was measured as described in Example 4. FIG. 21C shows sections of animals treated with aldose reductase siRNA showed significantly reduced red fluorescence compared to control and scrambled siRNA indicating that inhibition of aldose reductase prevented the reactive oxygen species production there by inhibiting the progression of tumor growth.

### EXAMPLE 7

### Inhibition of Aldose Reductase Prevents Proliferation of Breast Cancer Cells

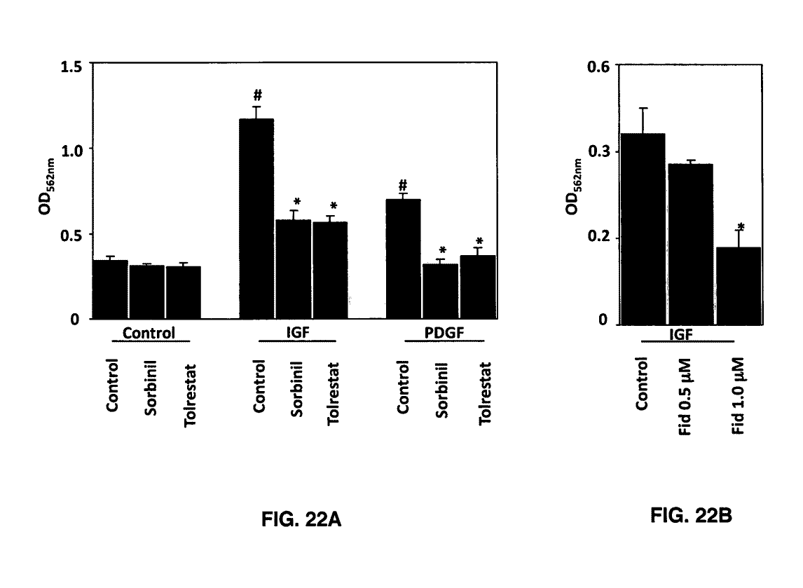

The potential of aldose reductase inhibitor as therapeutic agents to treat breast cancer was investigated and demonstrated. FIG. 22A show that aldose reductase inhibitors, sorbinil and tolrestat, is able to prevent growth factor induced cell growth in MCF-7 breast cancer cells. Similarly, FIG. 22B demonstrates that aldose reductase inhibitor fidarestat is able to prevent growth factor induced cell growth in MCF-7 breast cancer cells.

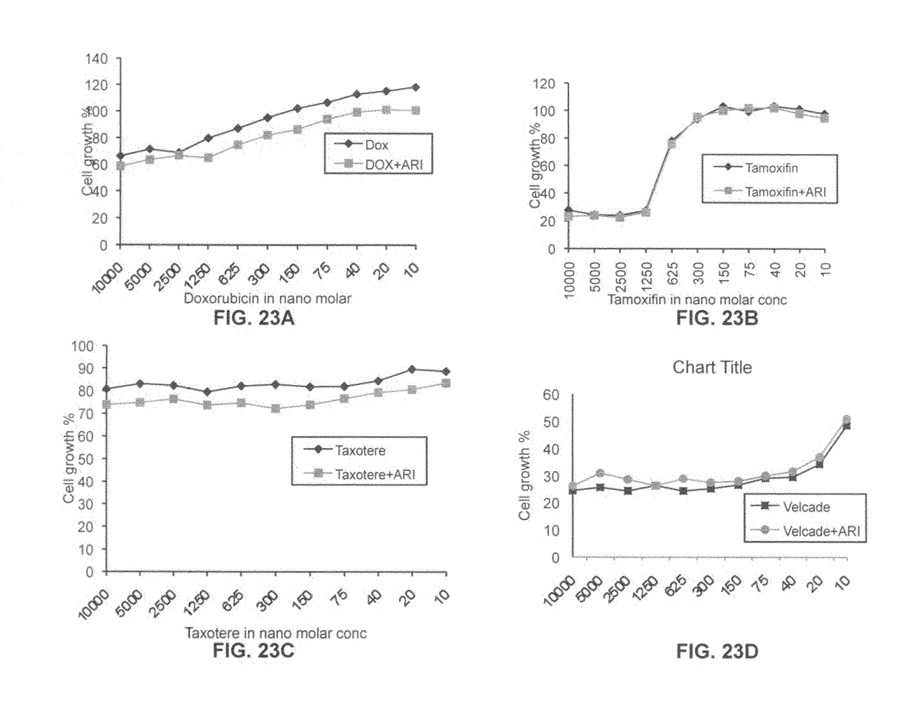

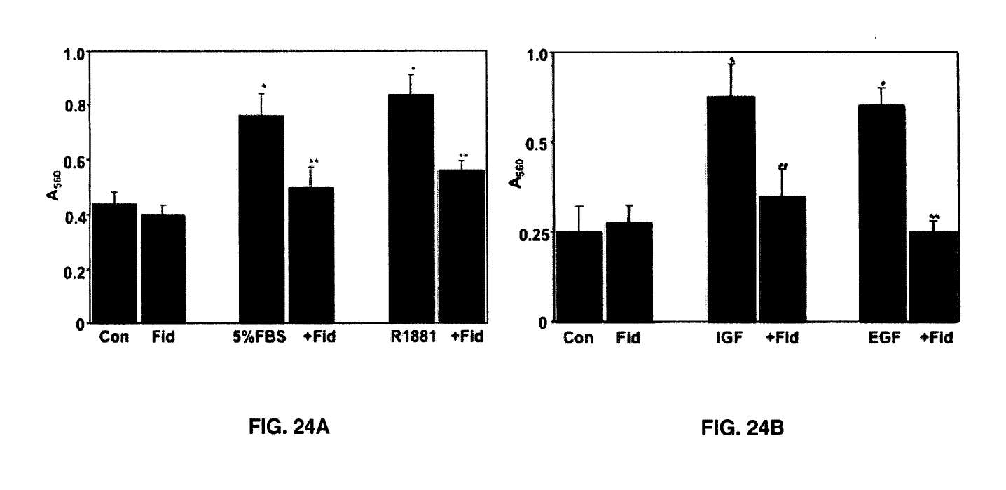

Most breast tumors are initially estrogen-dependent and thus responsive to estrogen ablation therapy, so estrogen deprivation is a standard therapeutic approach. However, breast cancer is heterogeneous in nature; tumors contain a population of both estrogen-dependent and -independent cells. Estrogen ablation therapy can successfully shrink primary and metastatic lesions by inducing apoptosis of estrogen-dependent BC cells. However, while ˜80% of patients initially respond to such therapy, this response is temporary in most patients, as tumor cells have adaptive mechanisms to become hormone-independent, and so resistant to estrogen ablation therapy. While there are several treatment approaches to hormone-refractory disease, many patients ultimately succumb. Therefore, successful new strategies are required for the eradication of both estrogen-dependent and -independent (FIGS. 23A-24D) breast cancer cells.

Estrogen independent breast cancer cells (MBA-MD-231) were treated with and without aldose reductase inhibitor (fidarestat 5 uM) in presence of various chemotherapeutic drugs such as doxorubicin, docetaxel (Taxotere), tamoxifen and bortezomib (Velcade) for 72 hrs. Cell growth was measured by MTT assay. Results demonstrate that doxorubicin and taxotere inhibited the cell growth significantly in presence of aldose reductase inhibitors, fidarestat compared to chemotherapeutic drugs alone. However inhibition is minimal in the other two tested chemotherapeutic drugs, Velcade and Tamoxifin. The inhibitory concentration values of drugs (IC50 values) needed for observed growth inhibition shows that fidarestat reduces five and one fold less doxorubicin and taxotere drugs respectively.

TABLE 5
Aldose reductase inhibitor prevents hormone
insensitive breast cancer cell growth

| | Without ARI | With ARI |
|---|---|---|
| Doxorubicin | 625 | 120 |
| Velcade | 32 | 22 |
| Tamoxifin | 1025 | 1010 |
| Taxotere | 2500 | 1420 |

### Preventing Metastasis in Breast Cancer Patients

A major strategy required to counter and treat breast cancer is prevention of metastasis which include dissociation and intravasation of cells from a primary tumor into the circulation, and survival there; arrest in small vessels in cell type-specific distant organs, allowing adhesion to endothelial cells and extravagation into surrounding tissues; proliferation to form a secondary (metastatic) tumor; and vascularization of that tumor. The progression of metastasis occurs via the blood or lymphatic systems, or both. Altered expression of proteases, adhesion molecules, growth factors, cytokines, chemokines and angiogenic factors regulated by reactive oxygen species (ROS)-induced transcription factors NF-kB, AP-1 and others are known to play significant role in tumor initiation and metastasis. We describe here the development of new drugs that can effectively treat breast cancer and its metastasis.

Recently, there have been a number of studies focusing on numerous therapeutic agents which have antioxidant properties. One limitation of these antioxidants is that to date, there is no suitable antioxidant with hydrophilic and hydrophobic properties available for delivery to cells. At adequate dosages and upon prolonged use, most antioxidants actually act as pro-oxidants.

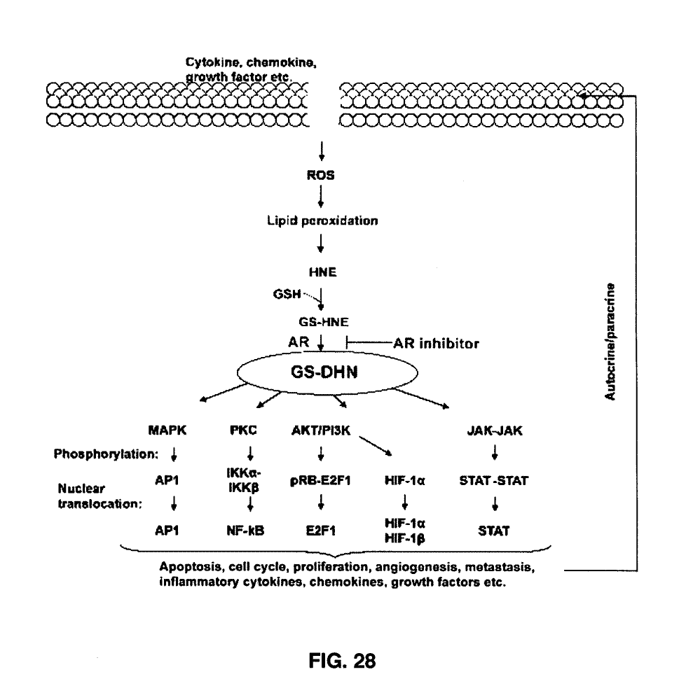

As we understand, carcinogenesis is mediated by oxidative stress, which occurs when the formation of oxidants overwhelms the cellular antioxidant capacity, leading to lipid peroxidation and the formation of lipid aldehydes. The lipid aldehydes readily conjugate with glutathione (2-5 mM in different tissues), catalyzed by glutathione-S-transferases (one of the most abundant enzymes in various tissues). The lipid aldehyde-glutathione conjugates are further reduced by aldose reductase. The intracellular concentration of these lipid aldehydes is tightly regulated by RaI-binding proteins (RLIP), which actively exports the conjugates. We have demonstrated that aldose reductase besides reducing aldo-sugars, reduces lipid aldehydes and their glutathione conjugates to corresponding alcohols such as HNE to DHN and GS-HNE to GS-DHN. The reduced lipid aldehyde-glutathione conjugate (GS-DHN) thus formed mediates the ROS signals for the activation of various kinases (PKC, PI3K, JAK-STAT) and phospholipases (PLC), which in turn control the expression of various inflammatory markers and growth factors that cause cell proliferation, apoptosis, angiogenesis and metastasis (FIG. 28). Although inhibitors of PKC, NF-kB, PLC, VEGF, HIF-1 alpha etc have been developed for breast cancer therapy, none of these drugs is very effective on long-term use.

We envision that inhibitors of aldose reductase could prevent the formation of reduced lipid aldehyde-glutathione conjugates such as GS-DHN, and so could prevent ROS signaling. Since decreasing ROS, which are upstream of various kinases and lipases, would be excellent for the treatment and/or prevention of breast cancer (including metastasis), drugs that could inhibit aldose reductase or block the formation of GS-DHN would be ideal. The biochemical and molecular pathways that change hormone sensitive breast cancer cells to hormone-resistant rendering them to ineffective hormone ablation therapy and that make small percentage of breast cancer CSCs initiates secondary tumors and metastasis. Since antioxidant enzymes such as aldose reductase have been overexpressed in such conditions and our studies have shown that aldose reductase mediates ROS-initiated inflammatory response, aldose reductase inhibitors should be novel therapeutic drugs for breast cancer prevention and chemotherapy.

This hypothesis is strongly supported by the fact that inflammatory markers induced by lipopolysaccharide (LPS) are inhibited 70-90% by aldose reductase inhibition/ablation in experimental animals (64). Similarly, we have shown that in colon cancer (cellular and animal models), aldose reductase inhibition significantly inhibited (by 70-80%) secretion of growth factors, cytokines, chemokines, and activation of cox-2 and iNOS (62, 63, 65). In fact, cox-2 inhibitors are currently in clinical use for prevention and therapy of various forms of cancer, but with limited success (66). Use of aldose reductase inhibitors to prevent breast cancer is further supported by preliminary studies showing that aldose reductase inhibition has profound effects on signaling pathways critical for tumor progression, and that aldose reductase inhibition prevents growth of human breast cancer cells in culture. Further, inhibiting aldose reductase prevents activation of aromatase in ER+ cells. Despite these observations, the role of aldose reductase in oxidant-signaling pathway(s) is not clearly understood. Initial findings indicate that inhibition of aldose reductase prevents the activation of the cAMP-dependent transcription factor CREB, which induces transcription of aromatase (67, 68), the enzyme that converts androgen to estrogen in ER+ BC cells. Further, the NF-kB/Cox-2/PGE2 pathway also appears to be involved in the activation of aromatase (69, 70). However, it is not known how aldose reductase inhibition blocks aromatase activity in ER+ cells; therefore the molecular mechanisms of aromatase activation were investigated in ER+ cells. Preliminary studies also suggest that aldose reductase inhibition prevents growth of ER− cells; however the mechanisms are not clearly understood. Aldose reductase inhibition attenuates the action of cell survival transcription factors such as NF-kB and its downstream signals, leading to cell death. Because chronic (˜52-week) administration of oral aldose reductase inhibitor is presently used clinically for prevention of diabetic complications in Japan, and has already undergone Phase III clinical trials in the US for diabetic neuropathy (71), these findings suggest that chronic aldose reductase inhibition by aldose reductase inhibitors such as fidarestat should be a safe and effective approach for the prevention and therapy of breast cancer growth and metastasis.

Increased ROS generation due to chronic inflammatory diseases or other oxidant stimuli is also associated with an elevated risk of breast cancer; ROS can drive carcinogenesis by altering cellular and molecular targets and pathways that are crucial to normal tissue homeostasis (72, 73). Despite multiple studies showing that ROS and oxidative stress are significant components of breast cancer, the mechanisms by which ROS regulate cell functions or induce tissue injury remain unclear. Previous studies show remarkable and unexpected metabolic regulation of inflammatory signaling by aldose reductase, and that the aldose reductase inhibitor fidarestat could prevent estrogen-dependent (MCF-7) and estrogen-independent (MDA-MB-231) human breast cancer cell growth. Moreover, inhibition of AR prevents aromatase activity in hormone-dependent cells and increases the expression of ER-α in estrogen-independent cells. Nonetheless, the molecular mechanisms by which aldose reductase regulates the signaling mechanisms remain unclear. Aldose reductase inhibitors are unique, as they could prevent both formation of inflammatory markers and their effects on cancer progression by blocking inflammatory markers signaling that activates transcription factors and causes tumorigenesis and metastasis.

### EXAMPLE 8

### Inhibition of Aldose Reductase Prevents Proliferation of Prostate Cancer Cells

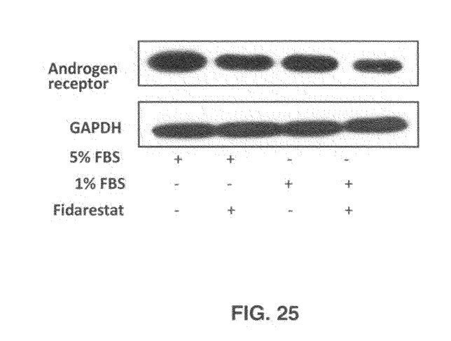

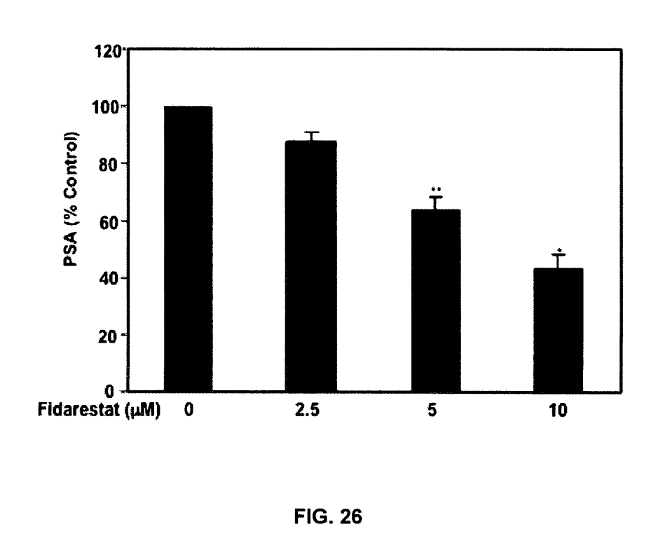

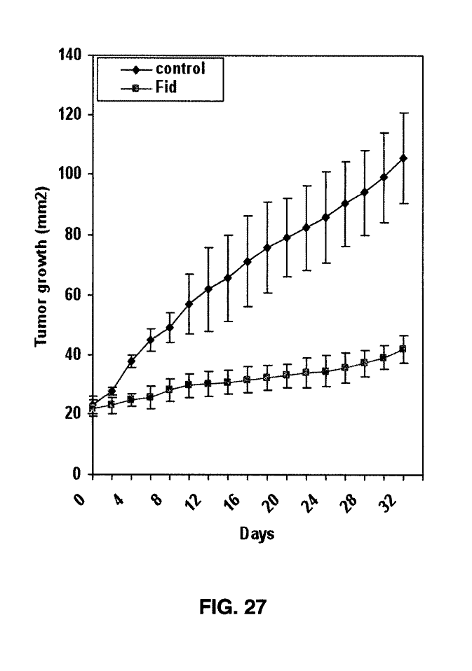

The potential of aldose reductase inhibitor as therapeutic agents to treat prostate cancer was demonstrated. Results (FIGS. 24A-24B) show that inhibition of aldose reductase by fidarestat significantly (>80%) prevented the 5% fetal bovine serum- and synthetic androgen (R1881)—induced proliferation in LNCaP cells and GFs such as EGF- and IGF-I-induced proliferation of PC3 cells. These results suggest that aldose reductase inhibition could be used to inhibit the growth of both androgen-dependent and -independent prostate cancer cells. Since androgen receptor plays an important role in the proliferation of androgen-dependent prostate epithelial cells and secretion of prostate specific antigen, it is important to measure the effect of aldose reductase inhibition on the expression of androgen receptor in LNCaP cells. As shown in the FIG. 25 fetal bovine serum (1% as wells as 5%) -induced expression of androgen receptor in LNCaP cells was significantly (50-60%) prevented by aldose reductase inhibition suggesting that aldose reductase could regulate the expression of androgen receptor. FIG. 26 shows that inhibition of AR significantly prevented the secretion of PSA in a concentration-dependent manner indicating that aldose reductase plays an important role in the secretion of PSA. Results also clearly show that prostate cancer PC3 cells tumor progression significantly (>60%) arrested in the animals treated with aldose reductase inhibitor, fidarestat, whereas uncontrolled growth was observed in the control mice (FIG. 27). These results suggest that inhibition of aldose reductase prevents in vivo tumor growth of prostate cancer cells.

### EXAMPLE 9

Inhibition of Aldose Reductase Prevents Human Colon Cancer Cell Invasion, Migration, Adhesion and in vivo Metastasis

Aldose reductase besides reducing aldo-sugars, efficiently catalyzes the reduction of lipid aldehydes such as 4-hydroxy-trans-2-nonenol (HNE) and their glutathione conjugates such as GS-HNE to 1,4-dihydroxynonene (DHN) and GS-DHN, respectively with low (μM) Kₘ compared to glucose with Kₘ in mM range (74, 75). We have shown earlier that inhibition of aldose reductase could prevent protein kinase C, NF-kB, and AP-1 activation and the increase in cell growth caused by HNE and GS-HNE, but not by GS-DHN (62, 63, 75). These studies suggested that the already reduced form of glutathione lipid aldehyde, GS-DHN is insensitive to aldose reductase inhibition and could be the main mediator of oxidative stress-induced NF-kB activation. Further, it was demonstrated that aldose reductase inhibition as well as ablation by siRNA could prevent the growth factors such as basic fibroblast growth factor (FGF)- and platelet-derived growth factor (PDGF)-induced activation of NF-kB, expression of Cox-2 and production of PGE2 in colon cancer cells (62, 63). Most remarkably, in nude mice xenograft model we have shown that inhibition of aldose reductase by ARsiRNA completely prevented growth of human colon adenocarcinoma cells (SW480) implanted subcutaneously (62). Recently, the results with azoxymethane (AOM) model in male BALB/c mice showed that inhibition of aldose reductase by pharmacological inhibitor of aldose reductase as well as aldose reductase gene knockout in mice significantly prevented aberrant crypt foci formation and AOM-induced expression of inflammatory markers, iNOS and Cox-2 and preneoplastic marker proteins, cyclin D1 and beta-catenin and activation of NF-kB in mice colons (65). However, the involvement of aldose reductase in the metastatic progression of human colon cancer is unknown.

The molecular mechanism by which aldose reductase inhibition prevents colon cancer metastasis was demonstrated. These in vitro results suggest that inhibition of aldose reductase could prevent cultured human colon cancer cells, HT29 adhesion, invasion and migration which are important steps for metastasis initiation. These results show that inhibition of aldose reductase could prevent metastatic growth of human colon cancer cells, injected in the spleen of nude mice, in the liver of nude mice. Thus, aldose reductase is an excellent therapeutic target for the prevention of metastatic spread of colon cancer.

### Materials and Methods

McCoy's 5A medium, MEM, DMEM-F12K, PBS, penicillin/streptomycin solution, trypsin, and fetal bovine serum (FBS) were purchased from Invitrogen (Carlsbad, Calif.). Antibodies against AR, Cyclin D1, MMP2, CD34, phospho-p65 and GAPDH were obtained from Santa Cruz Biotechnology, Inc. (Santa Cruz, Calif.). Sorbinil and zopolrestat were gift from Pfizer (New York, N.Y.). Fidarestat was obtained from Sanwa Kagaku Kenkyusho Co. Ltd. (Tokyo, Japan). Cell invasion and migration assay kits were obtained from Chemicon International Inc. (Billerica, Mass.). Epidermal growth factor (EGF), FGF and reagents used in Western blot analysis were obtained from Sigma (St. Louis, Mo.). AR-Sistable small interfering RNA was synthesized by Dharmacon Research (Chicago, Ill.). All other reagents used were of analytical grade.

### Cell Culture

Human colon cancer HT29 cells were obtained from American Type Culture Collection (ATCC; Manassas, Va.) and grown in McCoy's 5A medium supplemented with 10% FBS and 1% penicillin/streptomycin. KM20 cells were grown in minimum Eagle medium supplemented with 10% fetal bovine serum, 1% sodium pyruvate, 1% nonessential amino acids and 2% MEM essential vitamin. Human umbilical vascular endothelial cells (HUVEC) obtained from Cell Application Inc and grown in DMEM F-12K medium containing 10% FBS and cultured at 37° C. under an atmosphere containing 5% CO₂.

### Cell Invasion Assay

Invasive assay was performed using Basement Membrane Extract as per the manufacturer instructions (Chemicon International Inc., MA). Briefly, 50 μl of basal membrane extract (BME) solution was added on top of 8 micron polyethylene terephthalate membrane to each well of 96 well plate and incubated at 37° C. for 4 h to allow gel formation. HT29 or HT29-GFP cells at 20,000 cells/well in basal medium with EGF (5 ng/ml) or FGF (10 ng/ml) with or without sorbinil or zopolrestat (20 μM) were plated on Matrigel. After 24 h of incubation, invasion of cells towards bottom side of the well was measured using Calcein-AM florescent dye at 480/520 nm or photographed invaded cells by fluoresce microscope (100×).

### Cell Migration (Chemotaxis) Assay

HT29 cells were serum starved in McCoy's medium with or without sorbinil or zopolrestat (20 μM) for 24 h and cell migration assay was performed as per the manufacturer instructions (Chemicon International). Briefly, 2×10⁵ HT29 cells were plated per well of 24-well plate culture inserts containing 8.0-μm polycarbonate membrane. Subsequently, cells were treated with EGF (5 ng/ml) or FGF (10 ng/ml) with or without sorbinil or zopolrestat and the plate was transferred to 24-well plate which contained McCoy's medium with growth factor±sorbinil or zopolrestat and then the cells were incubated at 37° C. under a 5% CO₂ atmosphere. After 24 h, migrated cells were stained at bottom of the culture inserts and stain extracted into extraction buffer was measured calorimetrically at 560 nm. Migration of HUVEC in a scratch wound assay model was performed as described elsewhere (76).

### Cell Adhesion Assay

Cell adhesion assay was performed as described (28). Briefly, HUVEC were plated in 96-well plate at 3×10³ per well. Subconfluent cells were growth arrested in 0.1% FBS with or without aldose reductase inhibitor fidarestat (2 μM). After 24 h, EGF (5 ng/ml) or FGF (10 ng/ml) was added to the medium and the cells were incubated for another 24 h. HT29 cells were harvested in serum-free medium and applied at 2500 cells/well to the HUVEC monolayer in 96-well plate for 6 h. The non-adherent cells were removed by rinsing the wells with serum-free medium, adherent cells were quantified by MTT assay. For fluorescent microscopic analysis of cell adhesion, HT29 cells were labeled with PKH67 green fluorescent dye (PHK67ceIl linker kit, Sigma Co; USA) following manufacture instructions. Labeled HT29 cells were added to the growth factor-treated HUVEC, grown on chamber slides without and with presence and absence of aldose reductase inhibitors as described above and photographed using Nikon fluorescent microscope. The cell surface expression of adhesion molecules was measured by enzyme-linked immunosorbent assay by Butts et al (77).

### In Vivo Metastasis

Four to six weeks-old male nudenu/nu mice were obtained from Harlan (Indianapolis, Ind.) and experiments were approved by the UTMB Institutional Animal Care and Use Committee.

The mice were fed standard chow (Formula Chow 5008; Purina Mills, St. Louis, Mo.) and tap water ad libitum and allowed to acclimate for 1 week. Metastatic HT29-GFP or KM20-GFP cells were injected intrasplenically as described (3,4). Briefly, mice were anesthetized using isofluorane, a small left abdominal flank incision was created and the spleen was exteriorized. HT29-GFP or KM20-GFP cells were harvested using only trypsin and resuspended as a single-cell suspension in Hanks Balanced Salt Solution, free of Mg2+ and Ca2+. HT29-GFP or KM20-GFP cells (4×106 cells/100 μl) were injected into the spleen with a 27-gauge needle. The spleen was returned to the abdomen and the wound was closed in one layer with wound clips. After 24 h, spleen was removed and animals were randomized into metastatic control, scrambled ARSiRNA and aldose reductase inhibitor (sorbinil, fidarestat, and ARsiRNA) groups. Control group was fed with normal diet and AR inhibitor group fed with sorbinil (40 mg/kg body weight/day) in the diet whereas fidarestat (50 mg/kg body weight/day) was given in drinking water. In SiRNA treatment group, each mouse received Si-stable scrambled ARSiRNA or ARSiRNA (200 μg/mice) every 10 days for 3 times. Mice were killed after 35 days and metastasis in the liver was evaluated by using the Illumatool TLS (Lightools Research, Encinitas, Calif.).

### Western Blot Analysis

To examine the expression of AR, cyclin D1, MMP2, CD34, phospho-p65 and GAPDH proteins, Western blot analysis was carried out. Equal amounts of protein from liver tissue extracts were subjected to 12% SDS-PAGE followed by transfer of proteins to nitrocellulose filters and probing with the indicated antibodies. The antigen-antibody complex was detected by enhanced chemiluminescence (Pierce, Piscataway, N.J.).

### Reverse Transcription-PCR Analysis

Total RNA was isolated from liver samples by using RNeasy mini isolation kit (Qiagen, Valencia, Calif.). Total RNA (1.5 μg) sample was reverse transcribed with Omniscript and Sensiscript reverse transcriptase one-step reverse transcription-PCR (RT-PCR) system with HotStarTaq DNA polymerase (Qiagen) at 55° C. for 30 minutes followed by PCR amplification. The oligonucleotide primer sequences were as follows: 5′-ACCTGGATGCCGTCGTGGAC-3′ (sense) (SEQ ID NO: 8) and 5′-TGTGGCAGCACCAGGGCAGC-3′ (antisense) (SEQ ID NO: 9) for MMP2, 5′-TGTTTGCAAGCAGGACTTTG-3′ (sense) (SEQ ID NO: 10) and 5′-ACGTCAGCCTCCACACTCTT-3′ (antisense) (SEQ ID NO: 11) for cyclin D1, 5′-CCTGGAAGTCCCCTCCAGGGCAGG-3′ (SEQ ID NO: 12) and 5′-GGTTGAAGTTGGAGATGCCAATAGC-3′ (SEQ ID NO: 13) for aldose reductase and 5′-CGGAGTCAACGGATTTGGTCGTAT-3′ (sense) (SEQ ID NO: 14) and 5′-AGCCTTCTCCATGGTGGTGAAGAC-3′ (antisense) (SEQ ID NO: 15) for GAPDH. PCR was carried out in a GeneAmp 2700 thermocycler (Applied Biosystems, Foster City, Calif.) under the following conditions: initial denaturation at 95° C. for 15 min and 35 cycles of 94° C. for 30 sec, 62° C. for 30 sec, and 72° C. for 1 min and then 72° C. for 5 min for final extension. PCR products were electrophoresed in 2% Agarose-1x TAE gels containing 0.5 μg/mL ethidium bromide. Bands were quantified using Kodak Image Station 2000R loaded with Kodak one-dimensional image analysis software and the average fold change intensities were calculated.

### Immunohistochemical Analysis

KM20-GFP liver metastatic samples were perfusion-fixed with 4% paraformaldehyde and stored in 70% ethanol. Paraffin-embedded five-micrometer tumor sections were stained with antibodies against AR, cyclin D1, MMP2, CD34 and phospho-p65 using DakoCytomation LSAB+System-HRP kit.

### Statistical Analysis

Data are presented as mean±S.E. and the p values were determined using the unpaired Student's t test.

### Inhibition of Aldose Reductase Prevents Invasion and Migration of HT29 Cells

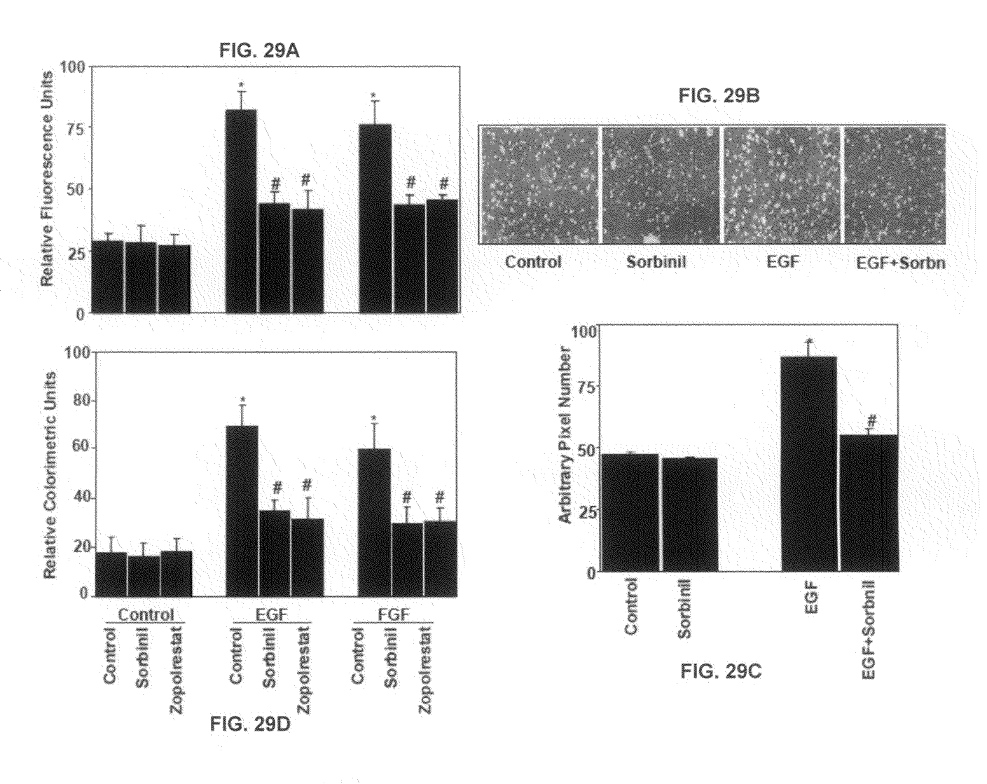

To understand the role of aldose reductase in the metastasis of colon cancer, we first determined the effect of aldose reductase inhibition on invasion and migration which play a pivotal role in the establishment of tumor progression and metastasis of colon cancer cells. Stimulation of HT29 cells with EGF or FGF for 16 h caused significant invasion through matrigel and inhibition of aldose reductase prevented invasive potential of metastatic HT29 cells by more than 70% (FIGS. 29A and 29B). Further, our results with in vitro migration of HT29 cells using transwell chemotaxis migration assay showed that inhibition of aldose reductase prevented EGF- as well as FGF-induced migration of HT29 cells through uncoated porous filters (FIG. 29D). These results indicate that aldose reductase inhibitors could prevent metastasis of colon cancer by inhibiting invasion and migration of colon cancer cells.

### Inhibition of Aldose Reductase Prevents Growth Factors-Induced Adhesion of Human Colon Cancer HT29 Cells to HUVEC

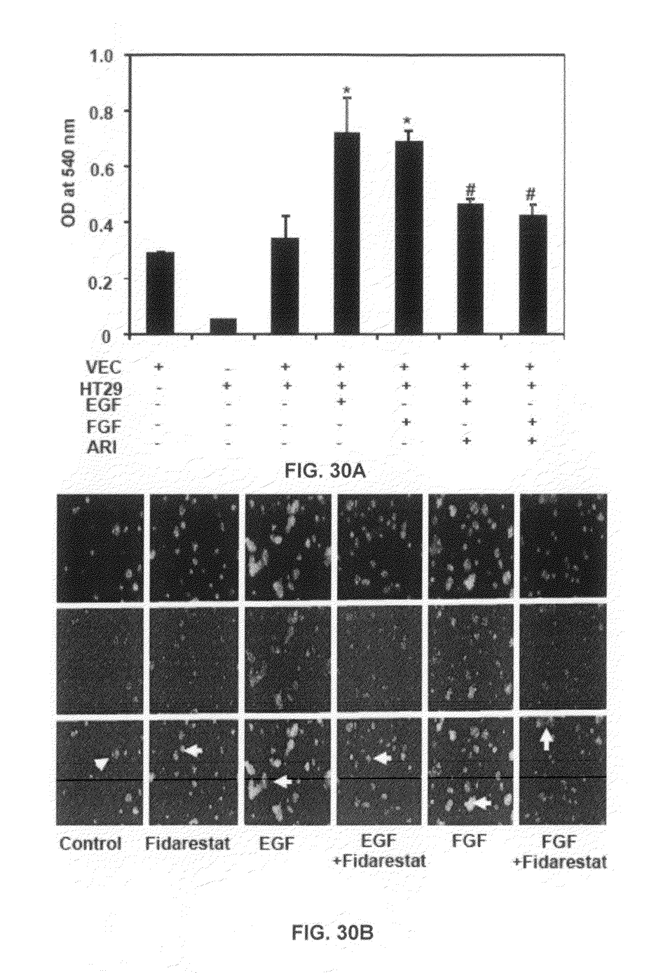

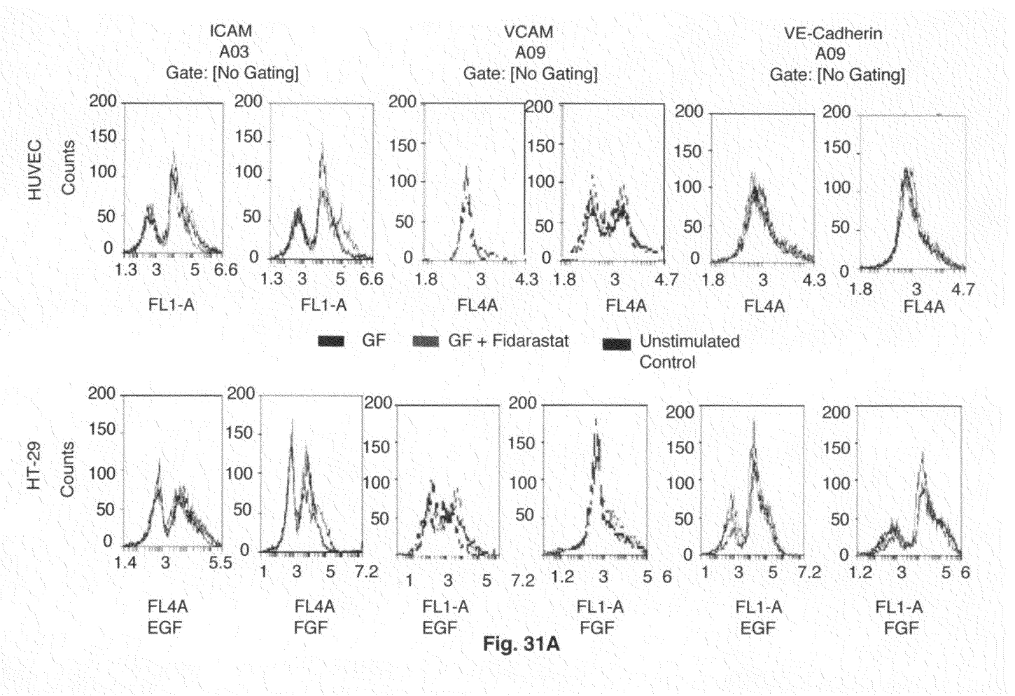

Since increased adhesion of cancerous cells to the endothelium is an important step to establish metastasis, we next determined the effect of aldose reductase inhibition on vascular adhesion of human colon cancer HT29 cells. As shown in the FIG. 30A stimulation of HUVEC with growth factors such as EGF or FGF caused approximately 60% increase in the adhesion of HT29 cells to HUVEC. However, pre-treatment of HUVEC cells with aldose reductase inhibitor, fidarestat significantly attenuated the adhesion of HT29 cells to HUVEC. These results were further confirmed by microscopic visualization of adhesion of fluorescent-labeled HT29 to HUVEC (FIG. 30B).

### Inhibition of Aldose Reductase Prevents Growth Factors-Induced Expression of Cell Surface Adhesion Molecules in HUVEC

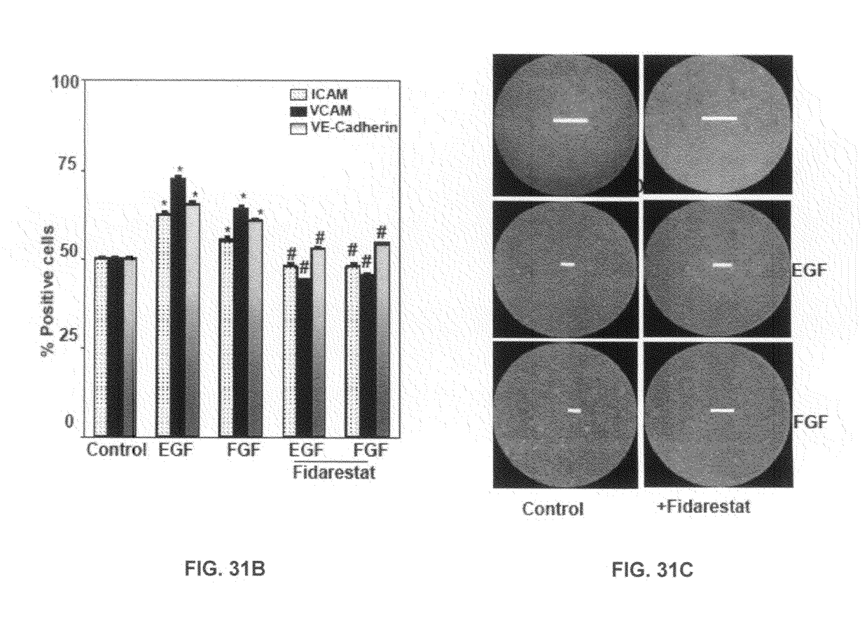

As shown in the FIG. 31A, stimulation of HUVEC with EGF or FGF for 24 h significantly increased the expression of VCAM, ICAM-1 and VE-cadherin compared to untreated HUVEC. The increased expression of adhesion molecules was significantly attenuated by fidarestat (FIGS. 31A-31B). However, aldose reductase inhibitor alone did not affect the basal levels of adhesion molecules. Since secretion of growth factors and cytokines by tumor cells is known to cause migration of endothelial cells which cause neovascularization (78), the effect of aldose reductase inhibition on growth factors-induced migration of HUVEC was determined. As shown in the FIG. 31C treatment of HUVEC with EGF or FGF caused pronounced migration of endothelial cells in wound model assay.

### Inhibition of Aldose Reductase Prevents Metastasis of Human Colon Cancer Cells in Nude Mice Liver

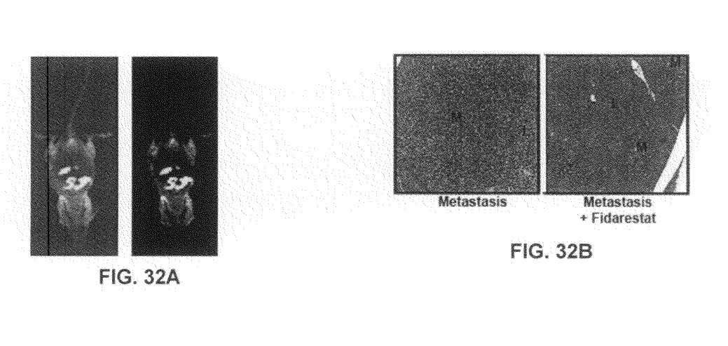

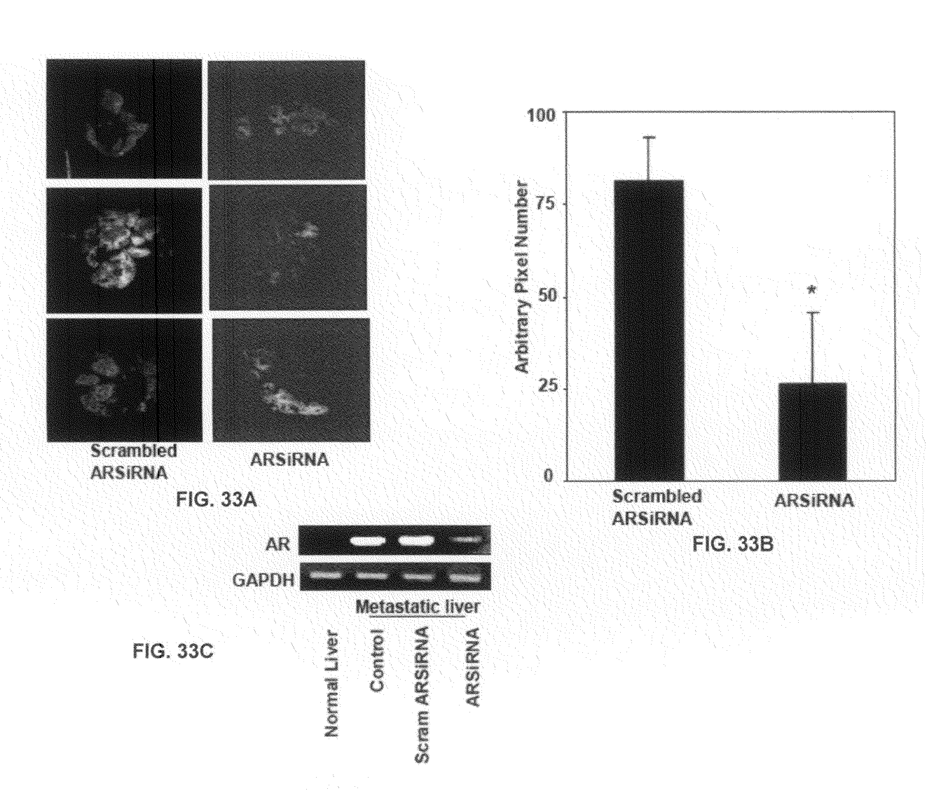

These in vitro studies clearly indicated that inhibition of aldose reductase could prevent invasion, migration and adhesion of HT29 cells which are critical steps that cause metastatic spread of tumor cells. Therefore, inhibition of aldose reductase could be useful to prevent metastasis in vivo. The effect of aldose reductase inhibition on the liver metastasis of colon cancer using athymic nude mice model was examined. In mice fed with control diet markedly increased metastases all over the liver was observed, whereas in mice given aldose reductase inhibitors or ARSiRNA fluorescence was significantly less (FIGS. 32A and 33A). The results were further quantified by measurement of fluorescence and values calculated as pixel numbers (FIGS. 32B and 33B). Results demonstrate a significant (60-67%) decrease in tumor metastasis in the sorbinil and fidarestat fed animals as well as ARSiRNA-treated group compared to control or aldose reductase scrambled SiRNA which correlates with our qualitative assessment. Similar results were observed in intrasplenical injection of GFP-transfected HT29 cells into the athymic mice. Further, RT-PCR analysis of metastatic liver suggested that ablation of aldose reductase by ARSiRNA almost completely (more than 90%) inhibited the expression of aldose reductase (FIG. 33C). Therefore, inhibition of aldose reductase could represent a unique strategy for the suppression of colorectal cancer metastasis.

The extent of tumor metastatic growth and changes in morphological architecture in liver were investigated by studying the histopathology of H&E stained liver sections. In control metastatic liver, multiple macroscopic foci of KM20-GFP cells were more compared to mice treated with aldose reductase inhibitor. Further, the largest dimension of metastasis and the percentage of the metastatic cancer tissue over the total liver parenchyma were significantly decreased in mice treated with aldose reductase inhibitor compared to untreated metastatic group (FIG. 32C). These results suggest that aldose reductase inhibition prevented metastatic growth in the liver.

### Inhibition of Aldose Reductase Prevents Expression of Metastatic Marker Proteins, NF-kB Activation and Histo-Pathological Changes in Nude Mice Liver

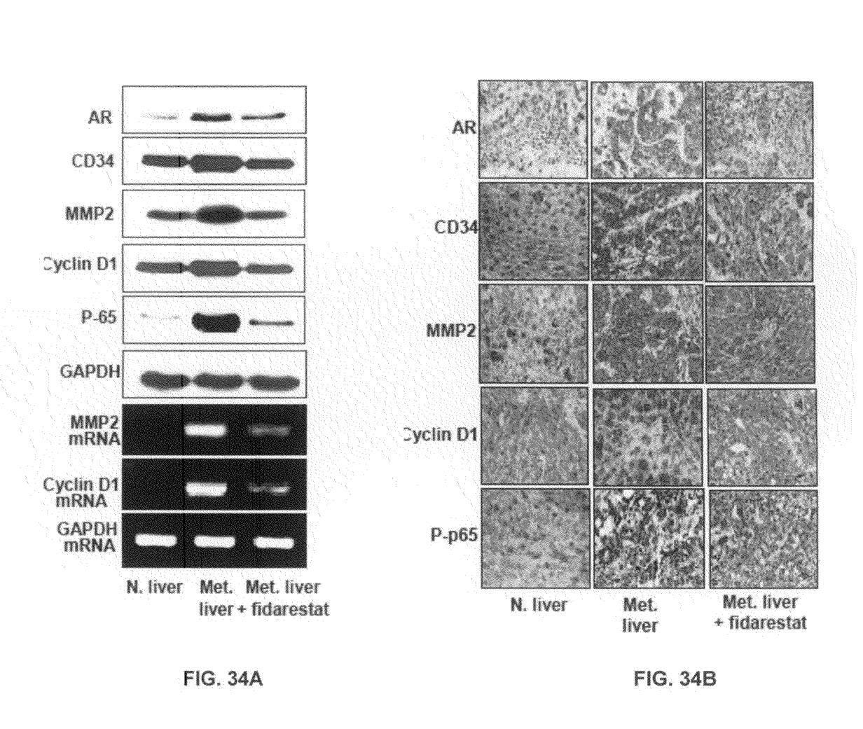

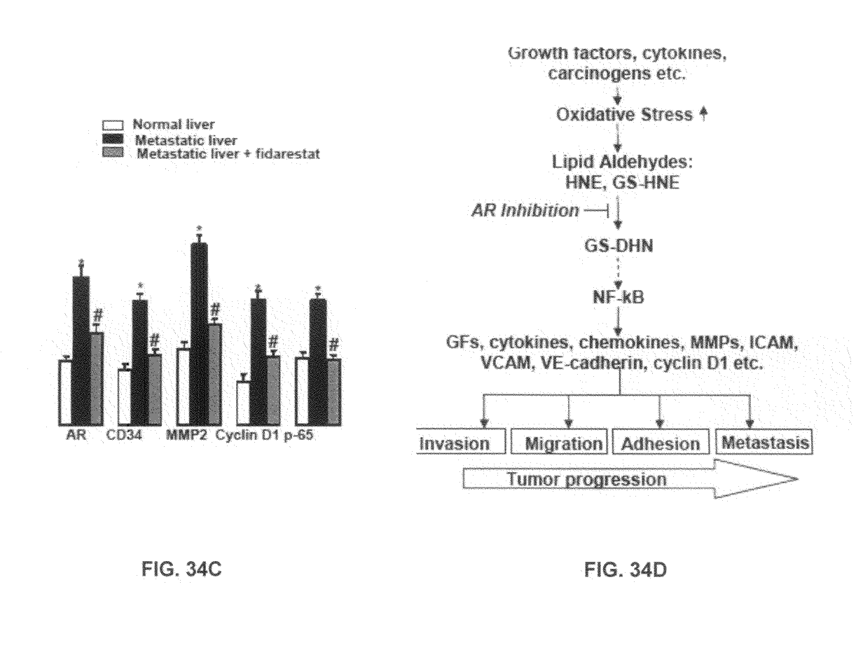

The expression of important proteins required for the metastatic growth of colon cancer was examined. The results showed a significant decrease in aldose reductase, MMP2, cyclin D1, CD34 expression and activation of p65 of NF-kB hetero-dimer in nude mice metastatic liver treated with fidarestat compared to untreated metastatic mice (FIG. 34A). Similar results were observed when IHC analysis of liver sections was performed with aldose reductase, CD34, MMP2, cyclin D1 and phospho-p65 antibodies (FIGS. 34B and 34C). Consistent with the protein levels, mRNA levels of cyclin D1 and MMP2 were significantly increased in metastastic liver, whereas treatment of mice with fidarestat significantly prevented the expression of MMP2 and cyclin D1 (FIG. 34A).

### Discussion

The mechanism of tumor metastasis involves a series of steps including escape of cells from primary tumor into blood or lymphatic system (intravasation), survival in the circulation and arrest in the secondary site (extravasation), vasucularization (angiogenesis) and growth of tumor (metastasis). In this progression of cancer any of the steps may become therapeutic target because inhibition of any one step would lead to disruption of entire metastatic cascade. The surgical resection and radiation therapy of primary colonic tumors are successful when tumor is locally confined. However, many of the patients are diagnosed when sub-clinical or clinical relevant liver metastasis has already occurred. During metastatic progression, dissociated cancer cells interact with various cellular components including blood cells and immune cells such as macrophages, lymphocytes and dendritic cells and induce embolization of blood vessels in the downstream organs. This mechanism is associated with generation of ROS and large quantities of pro-inflammatory cytokines, chemokines and growth factors. Indeed, involvement of ROS in cancer is well supported by epidemiological studies which have demonstrated that several forms of cancer progression can be slowed down/prevented by using antioxidants as chemopreventive agents. However, the mechanisms by which ROS regulate cell functions or induce tissue injury remain unclear. Our recent studies demonstrate that aldose reductase, plays a significant role in mediating ROS-induced inflammatory signals. Further, using colon cancer cellular and animal models have shown that aldose reductase is an obligatory mediator of oxidative stress/inflammation induced by growth factors, cytokines and carcinogens. In the present report it was demonstrated the involvement of aldose reductase in tumor cell adhesion, invasion, migration and metastasis using in vivo and in vitro models.

Tumor cell invasion and migration play a pivotal role in the progression of metastasis of cancer. Numerous studies with experimental metastasis models indicate that one of the most important component in the cell invasion is overexpression of proteases. Among the various proteases, MMPs are of particular importance because they degrade extracellular matrix and allow cells to overcome constrains of cell-cell and cell-matrix interaction. In addition, various reports show that expression of proteases is regulated by redox sensitive transcription factors such as NF-kB during oxidative stress- or inflammation-induced pathogenesis (14,35,36). This is further supported by various studies which have shown that antioxidants such as quercetin, resvervatol, and lycopene prevent the invasion/migration of tumor cells via inhibiting expression of MMPs and activation of PKC/AKT/PI3K/NF-kB. These results demonstrate that inhibition of aldose reductase significantly (70%) inhibits growth factor-induced invasion and migration of HT29 cells and increase in the expression of MMP2 in liver metastasis.

The successful progression of metastasis depends on the interaction of tumor cell with endothelial cell for the nutrients and oxygen and migration to other organs. Our in vitro results demonstrate that inhibition of aldose reductase prevents the growth factors such as EGF- and FGF-induced migration of human colon cancer HT29 cells and adhesion of human colon cancer HT29 cells to endothelial cells by inhibiting the expression of adhesion molecules such as ICAM, VCAM and VE-cadherin. Prevention of liver metastasis in nude mice by inhibition or ablation of aldose reductase which prevents the expression of the adhesion molecules suggest that aldose reductase is involved in adhesion of colon cancer cells to endothelial cells which would promote metastasis. Normally, human liver has extremely low levels of aldose reductase. However, aldose reductase is overexpressed in the liver under several oxidative stress and inflammation-related pathological conditions such as alcoholic liver cirrhosis, heart failure, myocardial ischemia, vascular inflammation, restenosis and cancer. Recently, Saraswat et al (79) demonstrated increased expression of aldose reductase in various cancerous tissues such as lung, breast, prostate, cervix, ovarian, colon, etc. Further, they showed that the specific activity of aldose reductase was higher in tumor areas than non-tumor regions of tissues. These results as well as our earlier studies demonstrating the prevention of tumor growth in nude mice by aldose reductase inhibition suggest that aldose reductase inhibitors could be excellent antitumor agents by preventing the processes required for metastasis (FIG. 34D).

Among the various experimental metastasis models intrasplenic injection of colon cancer cells produces reproducible, rapid and efficient liver metastasis. Injection of tumor cells via intravenous route produces few small experimental metastasis lesions in the lungs, but most of the mice macroscopic tumors have been found in various lymph nodes and the interscapular fat, but there has been no good correlation between lung tumor colony formation and the origin of the human colorectal cancer cells. The intrasplenic injection of colorectal carcinoma cells to nude mice provides a useful procedure to identify human colon cancer cells with metastatic potential to liver. A major disadvantage of experimental metastasis is that the progression stages of metastasis, i.e. pre-metastatic stages, cannot be studied, only the late stage of metastasis can be studied. Giavazzi et al (80) demonstrated that metastatic potential of freshly isolated colon cancer cells is achieved after instrasplenic injection and not after intra-venous, sub-cutaneous or intra muscular routes. Therefore, in present study we injected the green fluorescent-labeled human colon cancer cells intraspleenically to athymic nude mice and followed the metastasis in the liver after spleenectomy (80). These studies clearly demonstrate that inhibition of aldose reductase by two structurally different pharmacological inhibitors, sorbnil and fidarestat or by ablation of aldose reductase by SiRNA prevented the human colon cancer cells-induced metastatic growth in liver of nude mice. Thus, these results suggest that aldose reductase plays a pivotal role in the liver metastasis of the colon cancer.

These studies have demonstrated the critical role of aldose reductase in colon cancer cells migration, invasion and adhesion and the metastatic growth in liver. Since aldose reductase inhibitors such as fidarestat have already undergone phase-III clinical trials for the treatment of diabetic neuropathy and found to be safe without any major toxicity, these results may provide basis for the emerging preclinical and clinical investigations of aldose reductase inhibitors for the chemotherapeutic interventions of colon cancer progression and metastasis. The present studies also demonstrate that aldose reductase inhibitors could be excellent drugs for the prevention and therapy of colon cancer and may be other forms of cancer such as lung cancer, breast cancer and prostate cancer. Aldose reductase inhibitors are effective in the prevention of tumor growth as well as metastasis.

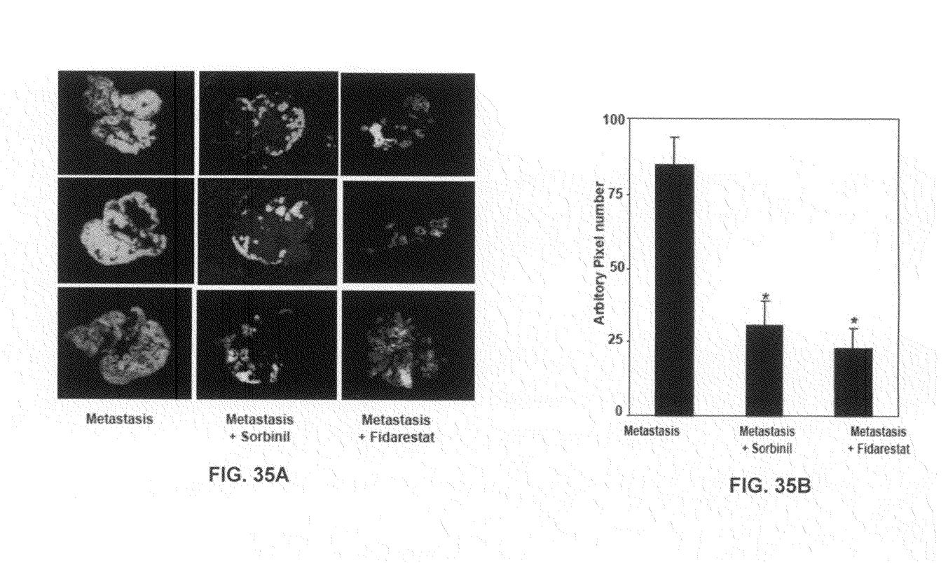

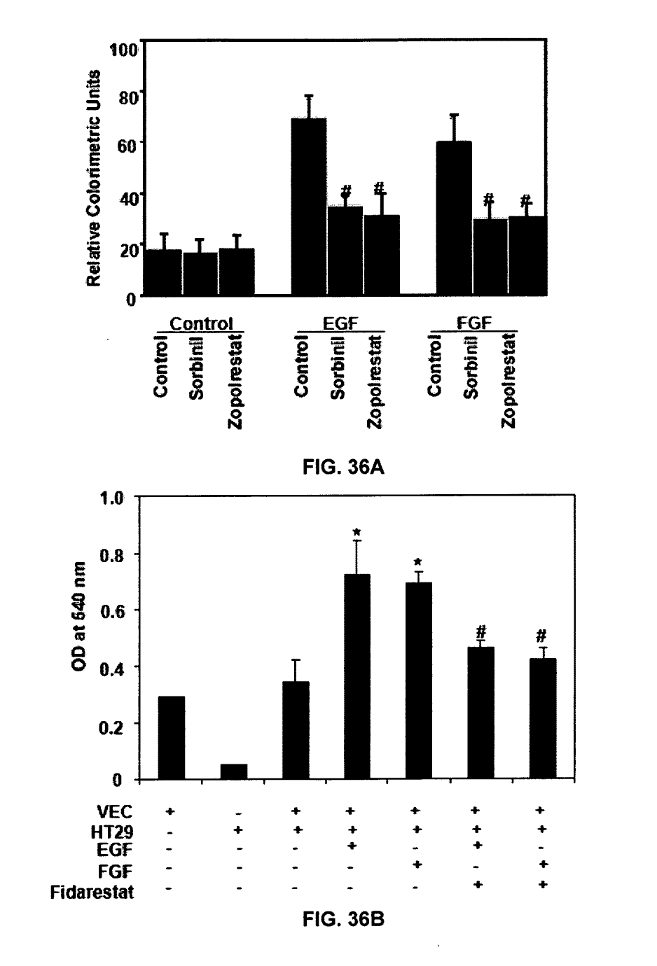

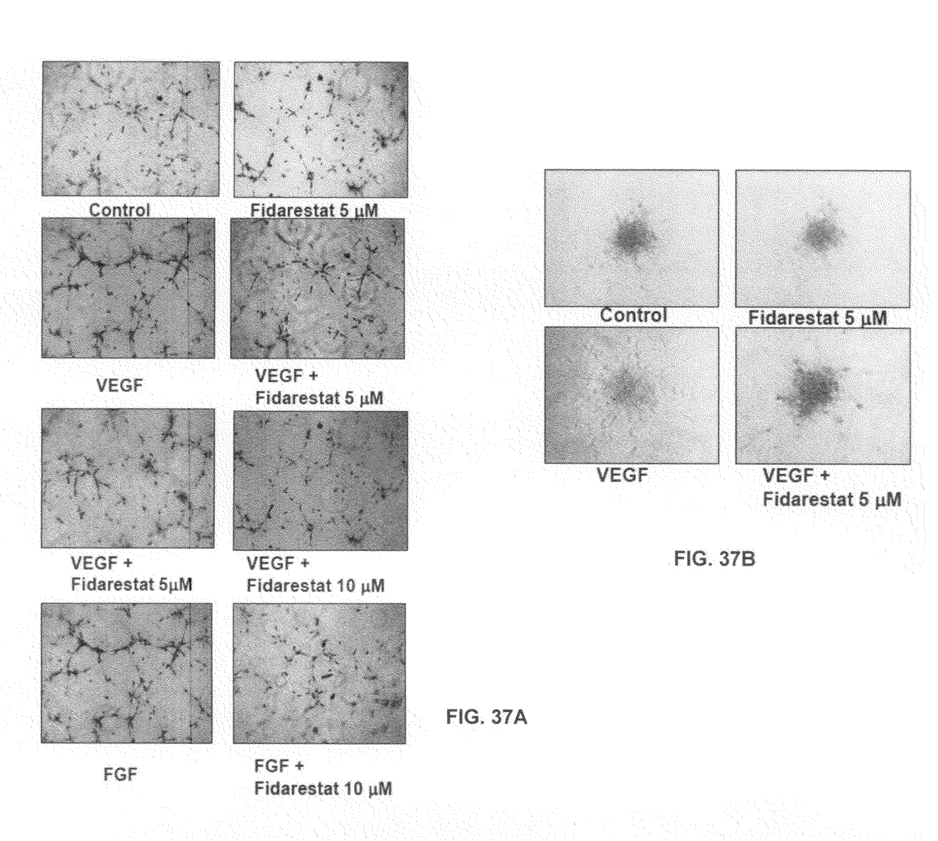

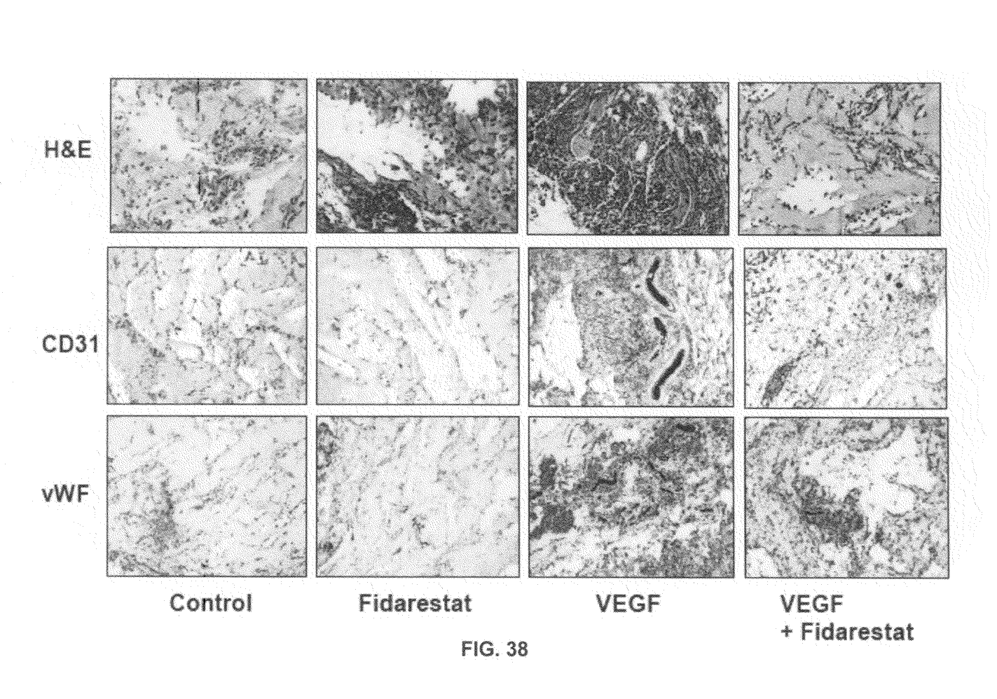

The present detailed studies have demonstrated that aldose reductase ablation or inhibition significantly prevents metastasis of colon cancer cells (FIG. 35A-35B). Aldose reductase inhibitors, sorbinil and fidarestat significantly inhibited colon cancer tumor cell adhesion, which detaches the cells from tumor and releases the cancer cells in circulation (FIG. 36A-36B), and invasion which allows the cancer cells to settle and grow in distant organs. Fidarestat also prevented adhesion of cancer cells in remote organs where otherwise the tumor cell would proliferate. In addition fidarestat prevented angiogenesis that is essential for primary as well as metastasized tumor growth (FIGS. 37A-37B & 38).

The following references were cited herein:
1. Jez et al. (1997) Biochem. J. 326: 625-636.
2. Rondeau et al. (1992) Nature 355:469-72.
3. Wilson et al. (1992) Science 257:81-84.
4. Bhatnagar et al. (1992) Biochem. Med. Metab. Biol. 48:91-121.
5. Nishikawa et al. (2000) Kidney Int. Suppl. 77:S26-30.
6. Parry, G. J. (1999) Am J Med 107:27S-33S.
7. Srivastava et al. (1995) Biochem. Biophys. Res. Commun. 217:741-746.
8. Srivastava et al. (1998) Biochem. J. 329:469-475.
9. Srivastava et al. (1999) Biochemistry 38:42-54.
10. van der Jagt et al. (1992) J. Biol. Chem. 267:4364-4369.
11. Kawamura et al. (1999) Biochem Pharmacol 58:517-24.
12. Rittner et al. (1999) J Clin Invest 103:1007-13.
13. Shinmura et al. (2002) Circ Res 91:240-6.
14. Ruef et al. (2000) Arterioscler Thromb Vasc Biol 20:1745-52.
15. Ramana et al. (2002) J Biol Chem 277(35):32063-70.
16. Uchida, K. (2003) Prog Lipid Res 42:318-43.
17. Grimshaw, C. E. (1992) Biochemistry 31:10139-45.
18. Varnai et al. (1999) Proteins 37:218-27.
19. Dixit et al. (2000) J. Biol. Chem. 275:21587-21595.
20. Ramana et al. (2000) Biochemistry 39:12172-12180.
21. Ramana et al. (2004) FASEB J 18:1209-18.
22. Ramana et al. (2004) Diabetes 53:2910-2920.
23. Petrash et al. (1992) J. Biol. Chem. 267:24833-24840.
24. Matthews, B. W. (1962) J. Mol. Biol. 33:491-7.
25. Otwinowski, Z. & Minor, W. (1997) Meth. Enz. 276:307-326.
26. Kissinger et al. (2001) Acta Crystallogr D Biol Crystallogr 57:1474-9.
27. Brunger et al. (1998) Acta Crystallogr D Biol Crystallogr 54(Pt 5):905-21.
28. Scott, et al. (2004) J Biol Chem 279:27294-301.
29. Tickle et al. (1998) Acta Crystallogr D Biol Crystallogr 5 (Pt 2):243-52.
30. Tickle et al. (1998) Acta Crystallogr D Biol Crystallogr 54(Pt 4):547-57.
31. Tickle et al. (2000) Acta Crystallogr D Biol Crystallogr 56(Pt 4):442-50.
32. Brunger A. T. (1992) Nature 355:472-474.
33. McRee D. E. (1999) J Struct Biol 125:156-65.
34. Matthews et al. (1975) Acta Crystallogr A31:480-487.
35. Hynes T. R. & Fox, R. O. (1991) Proteins 10:92-105.
36. van Aalten et al. (1996) Jour. of Comp. Aided Mol. Design 10:255-262.
37. Laskowski et al. (1996) J Biomol NMR. 8:477-86.
38. Murshudov et al. (1999) Acta Crystallogr D Biol Crystallogr 55(Pt 1):247-255.
39. (1994) Acta Cryst. D 50:760-763.
40. Delano W. L. (2003) (Delano Scientific, San Carlos, Calif.).
41. Calderone et al. (2000) Acta Crystallogr D Biol Crystallogr 56(Pt 5):536-40.
42. Urzhumtsev et al. (1997) Structure 5:601-12.
43. El-Kabbani et al. (1998) Mol Vis 4:19.
44. Bohren et al. (1992) J Biol Chem 267:20965-70.
45. Prade et al. (1997) Structure 5:1287-.
46. Sussman et al. (1998) Acta Crystallogr D Biol Crystallogr 54:1078-84.
47. Yang et al. (1998) Biochemistry 37:17145-56.
48. Bousset et al. (2001) Biochemistry 40:13564-.
49. Harrop et al. (2001) J. Biol. Chem. 276:44993-5000.
50. Becker et al. (1998) Nat Struct Biol 5:267-71.
51. Epp et al. (1983) Eur J Biochem 133:51-69.
52. Karplus et al. (1989) Eur J Biochem 178:693-703.
53. Kanaoka et al. (1997) Cell 90:1085-95.
54. Wilson et al. (1993) PNAS 90:9847-51.
55. Ramana et al. (2004) FEBS Lett., 570(1-3):189-194.
56. Ramana et al. (2003) FASEB J. 17(2):315-317.
57. Smith et al. (2000) Eur. J. Cancer, 36(5):664-674.
58. Liu et al. (2003) Cancer Res. 63(13):3632-3636.
59. Chen et al. (2005) J Biol Chem., 280(16):16354-16359.
60. Gokmen-Polar et al. (2001) Cancer Res., 61(4):1375-1381.
61. Tsujii et al. (1998) Cell, 93(5):705-716.
62. Tammali et al. (2006) Cancer Res., 66:9705-13.
63. Tammali et al. (2007) Cancer Lett., 252(2):299-306.
64. Ramana et al. (2006) 114(17):1838-46.
65. Tammali et al. (2009) Carcinogenesis, 30(5):799-807.
66. Marnett et al. (1999) Trends Pharmacol Sci, 20:465-9.
67. Sofi et al. (2003) Breast Cancer Res Treat, 79(3):399-407.
68. Bulun et al. (2009) Ann NY Acad Sci., 1155:121-31.
69. Chen et al. (2007) Cancer Res., 67(18):8914-22.
70. Purohit et al. (2005) J Steroid Biochem Mol Biol. 94(1-3):167-72.
71. Hotta et al. (2001) Diabetes Care, 24(10):1776-82.
72. Gago-Dominguez et al. (2008) 68(2):107-14.
73. Benz et al. (2008) Rev Cancer, 8(11):875-9.
74. Srivastava et al. (2005) Endocr Rev, 26:380-92.
75. Ramana et al. (2006) J Biol Chem, 281:17652-60.
76. Liang et al. (2007) Nature Protocols, 2:329-33.
77. Butts et al. (2007) Nuclear Receptor Signaling, 5: 1-6.
78. Fidler et al. (2003) Nat Rev Cancer, 3:453-8.
79. Saraswat et al. (2006) Med Sci Monit, 12:CR525-9.
80. Giavazzi et al. (1986) Cancer Res, 46:1928-33.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. It will be apparent to those skilled in the art that various modifications and variations can be made in practicing the present invention without departing from the spirit or scope of the invention. Changes therein and other uses will occur to those skilled in the art which are encompassed within the spirit of the invention as defined by the scope of the claims.

What is claimed is:

1. A method of treating a pathophysiological state or symptoms thereof resulting from aldose reductase-mediated signaling in a cytotoxic pathway in a subject, comprising:
administering a pharmacologically effective amount of an inhibitor of aldose reductase to the subject thereby preventing aldose reductase mediated signaling.

2. The method of claim 1, further comprising:
administering a chemotherapeutic drug to said subject.

3. The method of claim 2, wherein said chemotherapeutic drug is doxorubicin, docetaxel, tamoxifin or bortezomib.

4. The method of claim 2, wherein said chemotherapeutic drug is administered prior to administering said aldose reductase inhibitor.

5. The method of claim 1, wherein the inhibitor is a small interfering RNA (siRNA).

6. The method of claim 5, wherein the siRNA comprises a vector effective to transfect a cell characteristic of the pathophysiological state.

7. The method of claim 6, wherein the cell is a breast cancer cell, prostate cancer cell, colon cancer cell, a lung cancer cell or a metastatic cancer cell derived therefrom.

8. The method of claim 5, wherein the siRNA has the sequence shown in SEQ ID NO: 1.

9. The method of claim 1, wherein the inhibitor is effective to inhibit reduction of a glutathione-aldehyde conjugate by aldose reductase.

10. The method of claim 9, wherein the inhibitor interacts with a glutathione binding domain, but does not block a carbonyl binding site, in an active pocket of an aldose reductase having a three-dimensional conformation determined by DCEG binding to AR:NADPH.

11. The method of claim 10, wherein the active pocket comprises three flexible loops A, B and C, wherein the inhibitor interacts with at least the C loop.

12. The method of claim 1, wherein the inhibitor is 3,4-dihydro-4-oxo-3-[[5-(trifluoromethyl)-2-benzothiazolyl]methyl]-1-phthalazineacetic acid, (S)-6-fluorospiro[chroman-4,4′-imidazolidine]-2,5′-dione, N-[(5-trifluoromethyl)-6-methoxy-1-naphthalenyl]thioxomethyl}-N-methylglycine, 3-(4-bromo-2-fluorobenzyl)-3,4-dihydro-4-oxo-1-phthalazineacetic acid, 5-[(Z,E)-β-methylcinnamylidene]-4-oxo-2-thioxo-3-thiazolideneacetic acid, 3-(4-bromo-2-fluorobenzyl)-7-chloro-3,4-dihydro-2,4-dioxo-1(2H)quinazoline acetic acid, 3,4-dihydro-3-oxo-4-[(4,5,7-trifluoro-2-benzothiazolyl)methyl]-2H-1,4-benz othiazine-2-acetic acid, N-[3,5-dimethyl-4-[(nitromethyl)sulfonyl]phenyl]-2-methylbenzeneacetamide, (2S,4S)-6-fluoro-2′,5′-dioxospiro(chroman-4,4′-imidazolidine)-2-carboxamide, 2-[(4-bromo-2-fluorophenyl)methyl]-6-fluorospiro[isoquinoline-4(1H),3′-pyrrolidine]-1,2′,3,5′(2H)-tetrone, 2R,4R-6,7-dichloro-4-hydroxy-2-methylchroman-4-acetic acid, 2R,4R-6,7-dichloro-6-fluoro-4-hydroxy-2-methylchroman-4-acetic acid, 3,4-dihydro-2,8-diisopropyl-3-oxo-2H-1,4-benzoxazine-4-acetic acid, d-2-methyl-6-fluoro-spiro(chroman-4′,4′-imidazolidine)-2′,5′-dione, 2-fluoro-spiro(9H-fluorene-9,4′-imidazolidine)-2′,5′-dione, 2,7-di-fluoro-spiro(9H-fluorene-9,4′-imidazolidine)-2′,5′-dione, 2,7-di-fluoro-5-methoxy-spiro(9H-fluorene-9,4′-imidazolidine)-2′,5′-dione, 7-fluoro-spiro(5H-indenol[1,2-b]pyridine-5,3′-pyrrolidine)-2,5′-dione, d-cis-6′-chloro-2′,3′-dihydro-2′-methyl-spiro-(imidazolidine-4,4′-4′H-pyrano(2,3-b)pyridine)-2,5-dione, spiro[imidazolidine-4,5′(6H)-quinoline]-2,5-dione-3′-chloro-7,′8′-dihydro-7′-methyl-(5′-cis), 3,4-dihydro-3-(5-fluorobenzothiazol-2-yl-methyl)-4-oxophthalazin-1-yl-acetic acid, 3-(5,7-difluorobenzothiazol-2-ylmethyl)-3,4-dihydro-4-oxophthalazin-1-yl-acetic acid, 3-(5-chlorobenzothiazol-2-ylmethyl)-3,4-dihydro-4-oxophthalazin-1-yl-acetic acid, 3-(5,7-dichlorobenzothiazol-2-ylmethyl)-3,4-dihydro-4-oxophthalazin-1-yl-acetic acid, 3,4-dihydro-4-oxo-3-(5-trifluoromethylbenzoxazol-2-yl-methyl)phthalazin-1-yl-acetic acid; 3,4-dihydro-3-(5-fluorobenzoxazol-2-ylmethyl)-4-oxophthalazin-1-yl-acetic acid; 3-(5,7-difluorobenzoxazol-2-ylmethyl)-3,4-dihydro-4-oxophthalazin-1-yl-acetic acid, 3-(5-chlorobenzoxazol-2-ylmethyl)-3,4-dihydro-4-oxophthalazin-1-y-lacetic acid, 3-(5,7-dichlorobenzoxazol-2-ylmethyl)-3,4-dihydro-4-oxophthalazin-1-yl-acetic acid.

13. The method of claim 1, wherein the pathophysiological state is breast cancer, prostate cancer, colon cancer or lung cancer.

14. A method of treating a pathophysiological state or symptoms thereof resulting from aldose reductase-mediated signaling in a cytotoxic pathway in a subject, comprising:
administering a pharmacologically effective amount of an inhibitor of aldose reductase to the subject thereby preventing aldose reductase mediated signaling, wherein the inhibitor further suppresses metastasis of the cancer to a metastatic cancer.

15. The method of claim 14, further comprising:
administering a chemotherapeutic drug to said subject.

16. The method of claim 15, wherein said chemotherapeutic drug is doxorubicin, docetaxel, tamoxifen or bortezomib.

17. The method of claim 15, wherein said chemotherapeutic drug is administered prior to administering said aldose reductase inhibitor.

18. The method of claim 14, wherein the cancer is a colorectal cancer or a breast cancer and the metastatic cancer is a liver cancer or a bone cancer.

19. A method of treating a cancer in a subject, comprising:
administering a pharmacologically effective amount of an aldose reductase small interfering RNA (siRNA) to the subject to inhibit cancer cell proliferation thereby treating the cancer.

20. The method of claim 19, wherein the siRNA further suppresses metastasis of the cancer to a metastatic cancer.

21. The method of claim 19, wherein the cancer is a colorectal cancer and the metastatic cancer is a liver cancer.

22. The method of claim 19, wherein the siRNA comprises a vector effective to transfect the cancer cell.

23. The method of claim 19, wherein the siRNA has the sequence shown in SEQ ID NO: 1.

24. The method of claim 19, wherein the cancer is breast cancer, prostate cancer, colon cancer or lung cancer.

25. A method of treating breast cancer or symptoms thereof resulting from aldose reductase-mediated signaling in a cytotoxic pathway in a subject, comprising:
administering a pharmacologically effective amount of an inhibitor of aldose reductase to the subject thereby preventing aldose reductase mediated signaling.

26. The method of claim 25, wherein the inhibitor is a small interfering RNA (siRNA).

27. The method of claim 26, wherein the siRNA comprises a vector effective to transfect a breast cancer cell.

28. The method of claim 26, wherein the siRNA has the sequence shown in SEQ ID NO: 1.

29. The method of claim 25, wherein the inhibitor is 3,4-dihydro-4-oxo-3-[[5-(trifluoromethyl)-2-benzothiazolyl]methyl]-1-phthalazineacetic acid, (S)-6-fluorospiro[chroman-4,4′-imidazolidine]-2,5′-dione, N-[(5-trifluoromethyl)-6-methoxy-1-naphthalenyl]thioxomethyl}-N-methylglycine, 3-(4-bromo-2-fluorobenzyl)-3,4-dihydro-4-oxo-1-phthalazineacetic acid, 5-[(Z,E)-β-methylcinnamylidene]-4-oxo-2-thioxo-3-thiazolideneacetic acid, 3-(4-bromo-2-fluorobenzyI)-7-chloro-3,4-dihydro-2,4-dioxo-1(2H)quinazoline acetic acid, 3,4-dihydro-3-oxo-4-[(4,5,7-trifluoro-2-benzothiazolyl)methyl]-2H-1,4-benz othiazine-2-acetic acid, N-[3,5-dimethyl-4-[(nitromethyl)sulfonyl]phenyl]-2-methylbenzeneacetamide, (2S,4S)-6-fluoro-2′,5′-dioxospiro(chroman-4,4′-imidazolidine)-2-carboxamide, 2-[(4-bromo-2-fluorophenyl)methyl]-6-fluorospiro[isoquinoline-4(1H),3′-pyrrolidine]-1,2′,3,5′(2H)-tetrone, 2R,4R-6,7-dichloro-4-hydroxy-2-methylchroman-4-acetic acid, 2R,4R-6,7-dichloro-6-fluoro-4-hydroxy-2-methylchroman-4-acetic acid, 3,4-dihydro-2,8-diisopropyl-3-oxo-2H-1,4-benzoxazine-4-acetic acid, d-2-methyl-6-fluoro-spiro(chroman-4′,4′-imidazolidine)-2′,5′-dione, 2-fluoro-spiro(9H-fluorene-9,4′-imidazolidine)-2′,5′-dione, 2,7-di-fluoro-spiro(9H-fluorene-9,4′-imidazolidine)-2′,5′-dione, 2,7-di-fluoro-5-methoxy-spiro(9H-fluorene-9,4′-imidazolidine)-2′,5′-dione, 7-fluoro-spiro(5H-indenol[1,2-b]pyridine-5,3′-pyrrolidine)-2,5′-dione, d-cis-6′-chloro-2′,3′-dihydro-2′-methyl-spiro-(imidazolidine-4,4′-4′H-pyrano(2,3-b)pyridine)-2,5-dione, spiro[imidazolidine-4,5′(6H)-quinoline]-2,5-dione-3′-chloro-7,′8′-dihydro-7′-methyl-(5′-cis), 3,4-dihydro-3-(5-fluorobenzothiazol-2-yl-methyl)-4-oxophthalazin-1-yl-acetic acid, 3-(5,7-difluorobenzothiazol-2-ylmethyl)-3,4-dihydro-4-oxophthalazin-1-yl-acetic acid, 3-(5-chlorobenzothiazol-2-ylmethyl)-3,4-dihydro-4-oxophthalazin-1-yl-acetic acid, 3-(5,7-dichlorobenzothiazol-2-ylmethyl)-3,4-dihydro-4-oxophthalazin-1-yl-acetic acid, 3,4-dihydro-4-oxo-3-(5-trifluoromethylbenzoxazol-2-yl-methyl)phthalazin-1-yl-acetic acid; 3,4-dihydro-3-(5-fluorobenzoxazol-2-ylmethyl)-4-oxophthalazin-1-yl-acetic acid; 3-(5,7-difluorobenzoxazol-2-ylmethyl)-3,4-dihydro-4-oxophthalazin-1-yl-acetic acid, 3-(5-chlorobenzoxazol-2-ylmethyl)-3,4-dihydro-4-oxophthalazin-1-y-lacetic acid, 3-(5,7-dichlorobenzoxazol-2-ylmethyl)-3,4-dihydro-4-oxophthalazin-1-yl-acetic acid.

30. A method of treating prostate cancer or symptoms thereof resulting from aldose reductase-mediated signaling in a cytotoxic pathway in a subject, comprising:
administering a pharmacologically effective amount of an inhibitor of aldose reductase to the subject thereby preventing aldose reductase mediated signaling.

31. The method of claim 30, wherein the inhibitor is a small interfering RNA (siRNA).

32. The method of claim 31, wherein the siRNA comprises a vector effective to transfect a prostate cancer cell.

33. The method of claim 31, wherein the siRNA has the sequence shown in SEQ ID NO: 1.

34. The method of claim 30, wherein the inhibitor is 3,4-dihydro-4-oxo-3-[[5-(trifluoromethyl)-2-benzothiazolyl]methyl]-1-phthalazineacetic acid, (S)-6-fluorospiro[chroman-4,4′-imidazolidine]-2,5′-dione, N-[(5-trifluoromethyl)-6-methoxy-1-naphthalenyl]thioxomethyl}-N-methylglycine, 3-(4-bromo-2-fluorobenzyl)-3,4-dihydro-4-oxo-1-phthalazineacetic acid, 5-[(Z,E)-β-methylcinnamylidene]-4-oxo-2-thioxo-3-thiazolideneacetic acid, 3-(4-bromo-2-fluorobenzyl)-7-chloro-3,4-dihydro-2,4-dioxo-1(2H)quinazoline acetic acid, 3,4-dihydro-3-oxo-4-[(4,5,7-trifluoro-2-benzothiazolyl)methyl]-2H-1,4-benz othiazine-2-acetic acid, N-[3,5-dimethyl-4-[(nitromethyl)sulfonyl]phenyl]-2-methylbenzeneacetamide, (2S,4S)-6-fluoro-2′,5′-dioxospiro(chroman-4,4′-imidazolidine)-2-carboxamide, 2-[(4-bromo-2-fluorophenyl)methyl]-6-fluorospiro[isoquinoline-4(1H),3′-pyrrolidine]-1,2′,3,5′(2H)-tetrone, 2R,4R-6,7-dichloro-4-hydroxy-2-methylchroman-4-acetic acid, 2R,4R-6,7-dichloro-6-fluoro-4-hydroxy-2-methylchroman-4-acetic acid, 3,4-dihydro-2,8-diisopropyl-3-oxo-2H-1,4-benzoxazine-4-acetic acid, d-2-methyl-6-fluoro-spiro(chroman-4′,4′-imidazolidine)-2′,5′-dione, 2-fluoro-spiro(9H-fluorene-9,4′-imidazolidine)-2′,5′-dione, 2,7-di-fluoro-spiro(9H-fluorene-9,4′-imidazolidine)-2′,5′-dione, 2,7-di-fluoro-5-methoxy-spiro(9H-fluorene-9,4′-imidazolidine)-2′,5′-dione, 7-fluoro-spiro(5H-indenol[1,2-b]pyridine-5,3′-pyrrolidine)-2,5′-dione, d-cis-6′-chloro-2′,3′-dihydro-2′-methyl-spiro-(imidazolidine-4,4′-4′H-pyrano(2,3-b)pyridine)-2,5-dione, spiro[imidazolidine-4,5′(6H)-quinoline]-2,5-dione-3′-chloro-7,′8′-dihydro-7′-methyl-(5′-cis), 3,4-dihydro-3-(5-fluorobenzothiazol-2-yl-methyl)-4-oxophthalazin-1-yl-acetic acid, 3-(5,7-difluorobenzothiazol-2-ylmethyl)-3,4-dihydro-4-oxophthalazin-1-yl-acetic acid, 3-(5-chlorobenzothiazol-2-ylmethyl)-3,4-dihydro-4-oxophthalazin-1-yl-acetic acid, 3-(5,7-dichlorobenzothiazol-2-ylmethyl)-3,4-dihydro-4-oxophthalazin-1-yl-acetic acid, 3,4-dihydro-4-oxo-3-(5-trifluoromethylbenzoxazol-2-yl-methyl)phthalazin-1-yl-acetic acid; 3,4-dihydro-3-(5-fluorobenzoxazol-2-ylmethyl)-4-oxophthalazin-1-yl-acetic acid; 3-(5,7-difluorobenzoxazol-2-ylmethyl)-3,4-dihydro-4-oxophthalazin-1-yl-acetic acid, 3-(5-chlorobenzoxazol-2-ylmethyl)-3,4-dihydro-4-oxophthalazin-1-y-lacetic acid, 3-(5,7-dichlorobenzoxazol-2-ylmethyl)-3,4-dihydro-4-oxophthalazin-1-yl-acetic acid.

35. A method of inhibiting metastasis of colon cancer, comprising:
administering a pharmacologically effective amount of an inhibitor of aldose reductase to the subject thereby preventing aldose reductase mediated signaling.

36. The method of claim 35, wherein the inhibitor is a small interfering RNA (siRNA).

37. The method of claim 36, wherein the siRNA comprises a vector effective to transfect a colon cancer cell.

38. The method of claim 36, wherein the siRNA has the sequence shown in SEQ ID NO: 1.

39. The method of claim 35, wherein the inhibitor is 3,4-dihydro-4-oxo-3-[[5-(trifluoromethyl)-2-benzothiazolyl]methyl]-1-phthalazineacetic acid, (S)-6-fluorospiro[chroman-4,4′-imidazolidine]-2,5′-dione, N-[(5-trifluoromethyl)-6-methoxy-1-naphthalenyl]thioxomethyl}-N-methylglycine, 3-(4-bromo-2-fluorobenzyl)-3,4-dihydro-4-oxo-1-phthalazineacetic acid, 5-[(Z,E)-β-methylcinnamylidene]-4-oxo-2-thioxo-3-thiazolideneacetic acid, 3-(4-bromo-2-fluorobenzyl)-7-chloro-3,4-dihydro-2,4-dioxo-1(2H) quinazoline acetic acid, 3,4-dihydro-3-oxo-4-[(4,5,7-trifluoro-2-benzothiazolyl)methyl]-2H-1,4-benz othiazine-2-acetic acid, N-[3,5-dimethyl-4-[(nitromethyl)sulfonyl]phenyl]-2-methylbenzeneacetamide, (2S,4S)-6-fluoro-2′,5′-dioxospiro(chroman-4,4′-imidazolidine)-2-carboxamide, 2-[(4-bromo-2-fluorophenyl)methyl]-6-fluorospiro[isoquinoline-4(1H),3′-pyrrolidine]-1,2′,3,5′(2H)-tetrone, 2R,4R-6,7-dichloro-4-hydroxy-2-methylchroman-4-acetic acid, 2R,4R-6,7-dichloro-6-fluoro-4-hydroxy-2-methylchroman-4-acetic acid, 3,4-dihydro-2,8-diisopropyl-3-oxo-2H-1,4-benzoxazine-4-acetic acid, d-2-methyl-6-fluoro-spiro(chroman-4′,4′-imidazolidine)-2′,5′-dione, 2-fluoro-spiro(9H-fluorene-9,4′-imidazolidine)-2′,5′-dione, 2,7-di-fluoro-spiro(9H-fluorene-9,4′-imidazolidine)-2′,5′-dione, 2,7-di-fluoro-5-methoxy-spiro(9H-fluorene-9,4′-imidazolidine)-2′,5′-dione, 7-fluoro-spiro(5H-indenol[1,2-b]pyridine-5,3′-pyrrolidine)-2,5′-dione, d-cis-6′-chloro-2′,3′-dihydro-2′-methyl-spiro-(imidazolidine-4,4′-4′H-pyrano(2,3-b)pyridine)-2,5-dione, spiro[imidazolidine-4,5′(6H)-quinoline]-2,5-dione-3′-chloro-7,′8′-dihydro-7′-methyl-(5′-cis), 3,4-dihydro-3-(5-fluorobenzothiazol-2-yl-methyl)-4-oxophthalazin-1-yl-acetic acid, 3-(5,7-difluorobenzothiazol-2-ylmethyl)-3,4-dihydro-4-oxophthalazin-1-yl-acetic acid, 3-(5-chlorobenzothiazol-2-ylmethyl)-3,4-dihydro-4-oxophthalazin-1-yl-acetic acid, 3-(5,7-dichlorobenzothiazol-2-ylmethyl)-3,4-dihydro-4-oxophthalazin-1-yl-acetic acid, 3,4-dihydro-4-oxo-3-(5-trifluoromethylbenzoxazol-2-yl-methyl)phthalazin-1-yl-acetic acid; 3,4-dihydro-3-(5-fluorobenzoxazol-2-ylmethyl)-4-oxophthalazin-1-yl-acetic acid; 3-(5,7-difluorobenzoxazol-2-ylmethyl)-3,4-dihydro-4-oxophthalazin-1-yl-acetic acid, 3-(5-chlorobenzoxazol-2-ylmethyl)-3,4-dihydro-4-oxophthalazin-1-y-lacetic acid, 3-(5,7-dichlorobenzoxazol-2-ylmethyl)-3,4-dihydro-4-oxophthalazin-1-yl-acetic acid.


SEQUENCE LISTING (15 sequences)

SEQ ID NO: 1 - DNA, 21 bp, artificial sequence
  aldose reductase siRNA
aatcggtgtctccaacttcaa

SEQ ID NO: 2 - DNA, 21 bp, artificial sequence
  scrambled sequence of siRNA control
aaaatctccctaaatcataca

SEQ ID NO: 3 - DNA, 10 bp, artificial sequence
  NF-kappaB binding consensus sequence
gggactttcc

SEQ ID NO: 4 - DNA, 19 bp, artificial sequence
  Cox-2 sense primer
aaacccactccaaacacag

SEQ ID NO: 5 - DNA, 20 bp, artificial sequence
  Cox-2 antisense primer
tcatcaggcacaggaggaag

SEQ ID NO: 6 - DNA, 20 bp, artificial sequence
  beta-actin sense primer
tgagaccttcaacaccccag

SEQ ID NO: 7 - DNA, 24 bp, artificial sequence
  beta-actin antisense primer
ttcatgaggtagtctgtcaggtcc

SEQ ID NO: 8 - DNA, 20 bp, artificial sequence
  MMP2 sense primer
acctggatgccgtcgtggac

SEQ ID NO: 9 - DNA, 21 bp, artificial sequence
  MMP2 antisense primer
tgtggcagcaaccagggcagc

SEQ ID NO: 10 - DNA, 20 bp, artificial sequence
  cyclin D1 sense primer
tgtttgcaagcaggactttg

SEQ ID NO: 11 - DNA, 20 bp, artificial sequence
  cyclin D1 antisense primer
acgtcagcctccacactctt

SEQ ID NO: 12 - DNA, 24 bp, artificial sequence
  aldose reductase sense primer
cctggaagtcccctccagggcagg

SEQ ID NO: 13 - DNA, 25 bp, artificial sequence
  aldose reductase antisense primer
ggttgaagttggagatgccaatagc

SEQ ID NO: 14 - DNA, 24 bp, artificial sequence
  GAPDH sense primer
cggagtcaacggatttggtcgtat

SEQ ID NO: 15 - DNA, 24 bp, artificial sequence
  GAPDH antisense primer
agccttctccatggtggtgaagac